(19) United States
(12) Patent Application Publication
(10) Pub. No.: US 2022/0125803 A1
(43) Pub. Date: Apr. 28, 2022

(54) METHODS FOR THE TREATMENT OF PERIMENOPAUSE AND MENOPAUSE

(71) Applicant: Praxis Precision Medicines, Inc., Cambridge, MA (US)

(72) Inventors: Carlos LOYA, Cambridge, MA (US); Nicholas DEMARTINIS, Cambridge, MA (US); Gabriel BELFORT, Cambridge, MA (US)

(21) Appl. No.: 17/436,547

(22) PCT Filed: Mar. 4, 2020
(86) PCT No.: PCT/US2020/020938

§ 371 Date: Sep. 3, 2021

Related U.S. Application Data
(60) Provisional application No. 62/813,498, filed on Mar. 4, 2019.

(51) Int. Cl. A61K 31/573; A61K 31/536; A61K 31/58; A61P 15/12

(52) U.S. Cl. CPC A61K 31/573; A61P 15/12; A61K 31/58; A61K 31/536

(57) ABSTRACT

The invention relates to methods of treating the symptoms of perimenopause or menopause using positive allosteric modulators γ-aminobutyric acid type A (GABA-A PAMs) receptor, including 3α-hydroxy-3β-mnethoxymethyl-21-(1′-imidazolyl)-5α-pregnan-20-one and salts thereof.

### CROSS REFERENCE TO RELATED APPLICATIONS

This application claims priority to U.S. Application No. 62/813,498, filed Mar. 4, 2019, which is hereby incorporated by reference in its entirety herein.

### BACKGROUND OF THE DISCLOSURE

Women are twice as likely as men to develop depression or anxiety disorders. During the menopause transition, women are at increased risk for depression, including new onset depression and relapse for women with a history of depression. There are many overlapping symptoms between major depressive disorder (MDD) and depression occurring during the menopause transition (“perimenopausal depression”). However, there are critical differences and many in the medical community consider perimenopausal depression a unique depression subtype. Despite its prevalence (estimates indicate 20% of perimenopausal women present with depressive symptoms) and unique symptom profile, there are no FDA-approved therapies for treating perimenopausal depression. In addition to mood symptoms, many perimenopausal women experience physical, cognitive, sexual and sleep symptoms. Thus, there is a need for effective treatments for perimenopausal depression and other symptoms of the menopause transition.

3α-Hydroxy-3β-methoxymethyl-21-(1′-imidazolyl)-5α-pregnan-20-one (Compound 1) is a synthetic neuroactive steroid. Its primary molecular target is the γ-aminobutyric acid type A (GABA-A) receptor, where it acts as a positive allosteric modulator (PAM) of channel function. The structural formula of Compound 1 appears below.

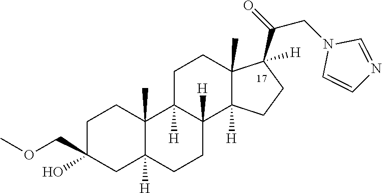

Neuroactive steroid GABA-A PAMs have demonstrated clinical efficacy in anesthesia, epilepsy, post-partum depression, and major depression.

### SUMMARY OF THE DISCLOSURE

The present disclosure, among other things, provides methods of treating the symptoms of perimenopause or menopause by administering a therapeutically effective amount of a GABA-A PAM. In some embodiments, the present disclosure provides methods of treating the vasomotor symptoms, sleep symptoms, cognitive symptoms, sexual symptoms and/or mood symptoms of perimenopause or menopause by administering a therapeutically effective amount of a GABA-A PAM. In some embodiments, the mood symptoms are selected from the group consisting of perimenopausal depression, irritability and anxiety. In some embodiments, the vasomotor symptoms are selected from the group consisting of hot flushes and night sweats. In some embodiments, the cognitive symptoms are selected from the group consisting of memory loss and difficulty concentrating.

In some embodiments, the GABA-A PAM is selected from the consisting of pregnanolone, allopregnanolone, allotetrahydrodeoxycorticosterone, ganaxolone, alphaxolone, alphadolone, hydroxydione, minaxolone, Althesin, Renanolone, SAGE-324 (Zuranolone), SAGE-689, SAGE-217 (3α-hydroxy-3β-methyl-21-(4-cyano-1H-pyrazol-1′-yl)-19-nor-50-pregnan-20-one), and any neuroactive steroid as described in U.S. Publication No. 2017/0240589.

In some embodiments, the GABA-A PAM is Compound 1 or a pharmaceutically acceptable salt thereof to a patient in need thereof. In some embodiments, the method comprises orally administering a daily dose of about 5 mg to about 120 mg of Compound 1 or a pharmaceutically acceptable salt thereof to a patient in need thereof.

In some embodiments, the patient in need of a treatment of the symptoms of perimenopause or menopause is a patient in perimenopause according to the Stages of Reproductive Aging Workshop+10 (STRAW+10) Staging System. In some embodiments, the patient is in early menopausal transition according to the STRAW+10 Staging System. In some embodiments, the patient is in late menopausal transition according to the STRAW+10 Staging System. In some embodiments, the patient is in early postmenopause according to the STRAW+10 Staging System. In some embodiments, the patient in need of a treatment of the symptoms of perimenopause or menopause is a patient in postmenopause according to the STRAW+10 Staging System.

In some embodiments, the patient in need of a treatment of the symptoms of perimenopause or menopause is a patient in perimenopause according to the Female Reproductive Lifecycle and Hormones Questionnaire (FRLHQ). In some embodiments, the patient is in early menopausal transition according to the FRLHQ. In some embodiments, the patient is in late menopausal transition according to the FRLHQ. In some embodiments, the patient is in early postmenopause according to the FRLHQ. In some embodiments, the patient in need of a treatment of the symptoms of perimenopause or menopause is a patient in postmenopause according to the FRLHQ.

In some embodiments, the present disclosure provides adjunctive treatment for treating the symptoms of perimenopause or menopause comprising administering an effective amount of a GABA-A PAM. In some embodiments, the patient in need of a treatment of the symptoms of perimenopause or menopause is a patient that is partially responsive to other therapies (such as hormone replacement therapy, SSRIs, and selective estrogen receptor modulator (SERM)).

In some embodiments, the patient in need of a treatment of the symptoms of perimenopause or menopause is a patient whose symptoms are refractory to other therapies.

In some embodiments, the present disclosure provides methods of treating the symptoms of perimenopause or menopause by administering a GABA-A PAM in combination with at least one additional therapeutic agent to a patient in need thereof. In some embodiments, the additional therapeutic agent is an antidepressant agent selected from selective serotonin reuptake inhibitors (such as paroxetine), serotonin norepinephrine reuptake inhibitors, tricyclic antidepressants, monoamine oxidase inhibitors, mirtazapine, bupropion, lamotrigine, and atypical antipsychotics. In some embodiments, the additional therapeutic agent is a hormone replacement therapy selected from an estrogen (such as estradiol, estradiol valerate, estradiol acetate, etc.) and a progestin (such as progesterone, medroxyprogesterone acetate, etc.), or a combination thereof (such as estradiol/norethindrone acetate, estradiol/drospirenone). In some embodiments, the additional therapeutic agent is an estrogen agonist/antagonist (such as ospemifene).

### DETAILED DESCRIPTION OF THE DRAWINGS

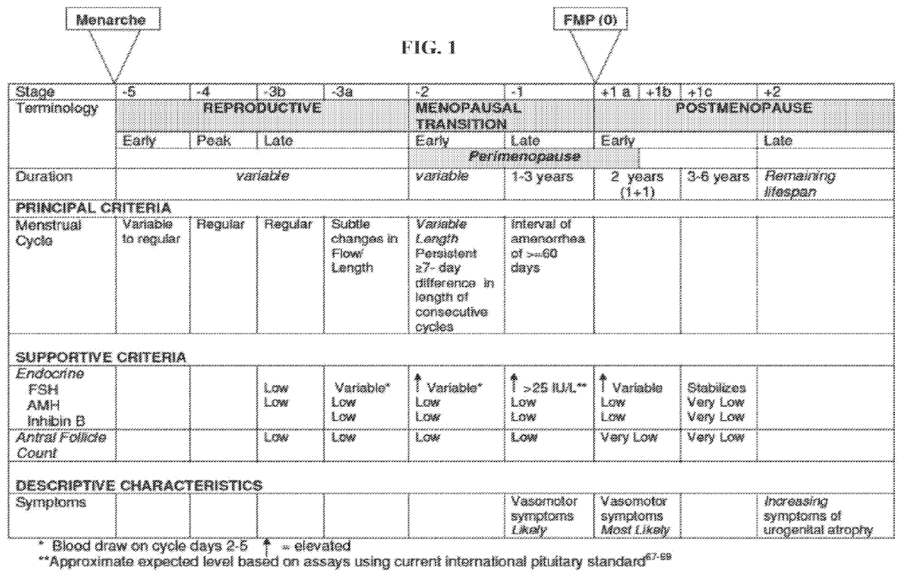

FIG. 1 shows the Stages of Reproductive Aging Workshop 10 (STRAW-10) Staging System.

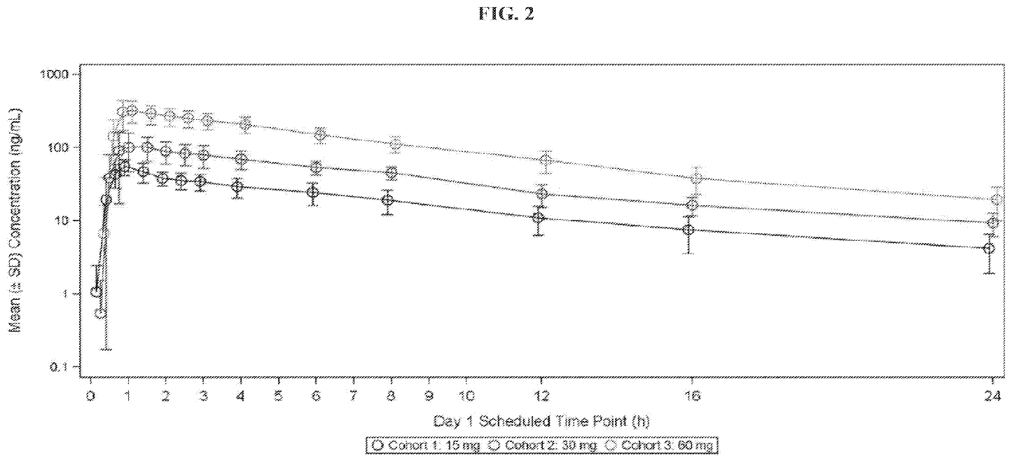

FIG. 2 is a graphical representation of the mean Compound 1 plasma concentration versus time for 15.0 mg daily administration of Compound 1 (Cohort 1), 30.0 mg daily administration of Compound 1 (Cohort 2), and a 60.0 mg daily administration of Compound 1 (Cohort 3).

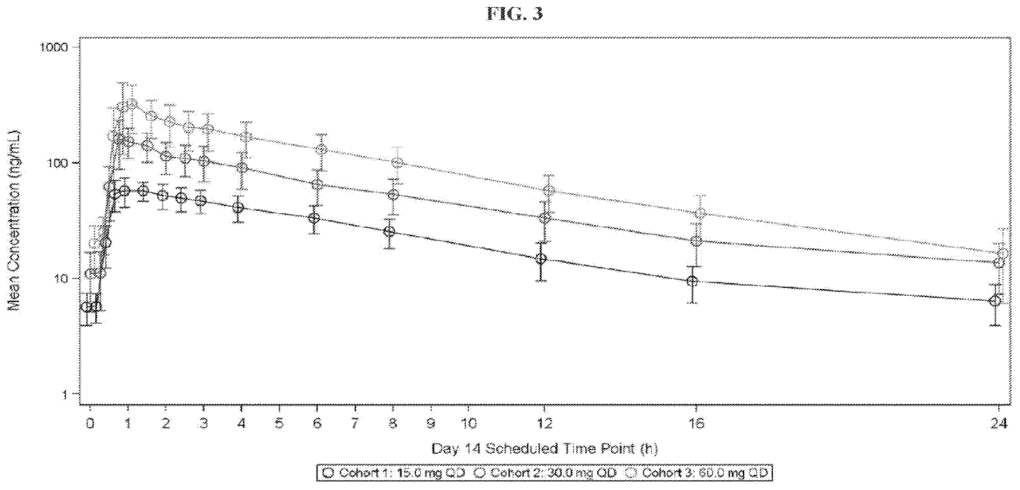

FIG. 3 a graphical representation of the mean Compound 1 steady state plasma concentration versus time for 15.0 mg daily administration of Compound 1 (Cohort 1), 30.0 mg daily administration of Compound 1 (Cohort 2), and a 60.0 mg daily administration of Compound 1 (Cohort 3).

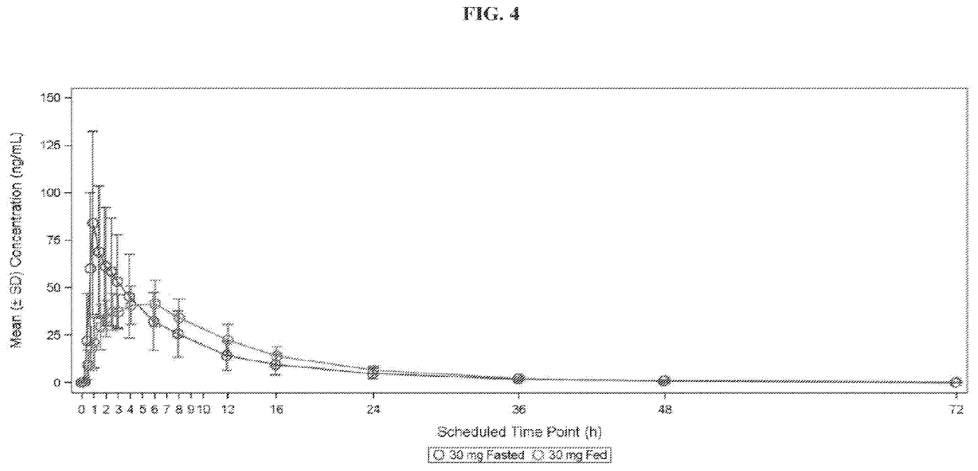

FIG. 4 is a graphical representation of the mean Compound 1 plasma concentration versus time for the 30 mg fed administration of Compound 1 and 30 mg fasted administration of Compound 1 (Cohort 4).

### DEFINITIONS

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The terms “administer,” “administering” or “administration” as used herein refer to either directly administering a compound or pharmaceutically acceptable salt or ester of the compound or a composition comprising the compound or pharmaceutically acceptable salt or ester of the compound to a patient.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical agent from one organ, or portion of the body, to another organ or portion of the body.

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound, or a salt, solvate or ester thereof, that, when administered to a patient, is capable of performing the intended result. For example, an effective amount of a salt of Compound 1 is that amount that is required to reduce at least one symptom of perimenopause in a patient. The actual amount that comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “menopause” as used herein refers to menopause as defined by a validated staging system. In some embodiments, “menopause” refers to menopause as defined by the Stages of Reproductive Aging Workshop+10 Staging System.

The phrase “perimenopause” as used herein refers to early and late menopause transition stages as well as early postmenopause as defined by a validated staging system. In some embodiments, “perimenopause” refers to early and late menopause transition stages as well as early postmenopause as defined by the Stages of Reproductive Aging Workshop 10 Staging System. In some embodiments, “perimenopause” refers to early and late menopause transition stages as well as early postmenopause as defined by the Female Reproductive Lifecycle and Hormones Questionnaire (FRLHQ). Throughout the present disclosure reference is made to the treatment of the symptoms of perimenopause. The present disclosure contemplates the treatment of the perimenopausal women as defined by the Stages of Reproductive Aging Workshop 10 Staging System as well as perimenopausal women as defined by as the FRLHQ.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salts” as used herein embraces pharmaceutically acceptable salts commonly used to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid.

The term “treating” as used herein with regard to a patient, refers to improving at least one symptom of the patient's disorder. Treating can be curing, improving, or at least partially ameliorating a disorder.

The term “therapeutic effect” as used herein refers to a desired or beneficial effect provided by the method and/or the composition. For example, the method for treating the symptoms of perimenopause or menopause provides a therapeutic effect when the method reduces at least one symptom of perimenopause or menopause (such as hot flushes or perimenopausal depression) in a patient.

### DETAILED DESCRIPTION OF THE DISCLOSURE

Perimenopause is the interval immediately preceding menopause, when women transition from a reproductive to non-reproductive state, until menopause, when menses have ceased for a period of at least 12 months. Women typically reach perimenopause in their mid- to late forties with a number of physical and mental health changes that persist for 4 to 5 years before menopause is reached. The Stages of Reproductive Aging Workshop (STRAW+10) criteria provide the framework for characterizing reproductive aging through reproductive stages and menopause (see, FIG. 1). According to STRAW+10, the perimenopausal period comprises the early and late menopausal transition phase as well as the early stage of menopause.

Women are twice as likely as men to develop depression or anxiety disorders. The menopause transition is a time when women are at increased risk for depression, including new onset depression and relapse for women with a history of depression. Perimenopause brings on a number of endocrine changes. Menstrual cycle changes typically commence with irregularities in cycle length and progress to menses cessation. The endocrine environment is highly erratic during perimenopause with fluctuations in hormonal concentrations, including follicle-stimulating hormone (FSH), estradiol, and progesterone. Without being bound by any theory, it is hypothesized that estradiol and progesterone fluctuations (which in turn (1) provides fluctuations in endogenous neurosteroids (e.g., allopregnanolone (“ALLO”), a positive allosteric modulator of GABA-A receptors) and (2) reduces GABA-A receptor activity) are in part responsible for the mood symptoms of perimenopause (Gordon, J., et al. Ovarian Hormone Fluctuation, Neurosteroids, and HPA Axis Dysregulation in Perimenopausal Depression: A Novel Heuristic Model, Am. J. Psychiatry, 172:3, March 2015, 227-236.).

An estimated 20% of perimenopausal women present to their primary care physicians with depressive symptoms. Although there are many overlapping symptoms between major depressive disorder (MDD) and depression occurring during the menopause transition (“perimenopausal depression”), there are critical differences such that many in the medical community consider perimenopausal depression a unique depression subtype. For example, MDD is characterized by sadness whereas the mood profile of perimenopausal depression includes anger, irritability and paranoia that may manifest as verbal outbursts often over minor stressor and out of character for women experiencing the symptoms. As such, unique rating scales, including Meno-D (discussed below) for measuring the severity of perimenopausal depression have been developed.

In addition to mood symptoms, many perimenopausal women experience physical (e.g., hot flushes, night sweats, physical pain, weight gain, and low energy), cognitive (such as problems with memory and concentration), sexual and sleep (e.g., insomnia and sleep disturbance) symptoms.

Despite its high incidence, there is no FDA-approved treatment for perimenopausal depression. Since the symptoms of perimenopausal depression have physical and mood symptoms that are distinct from those experienced by MDD patients, there is a need for methods of treating women experiencing the symptoms of perimenopause and menopause.

Neuroactive steroids (NASs) are a family of compounds (synthetic and naturally occurring) that affect neurophysiological functions through allosteric modulation of GABA-A receptors. The endogenous NASs allopregnanolone and pregnanolone are GABA-A PAMs that are dysregulated in mood disorders and show preclinical efficacy in animal models of anxiety and depression. NASs bind to a different binding site on the GABA-A receptor than benzodiazepines or the endogenous agonist GABA (Hosie A M, Wilkins M E, Da Silva H M A, Smart T G. Endogenous neurosteroids regulate GABA-A receptors through two discrete transmembrane sites. Nature. 2006; 444(7118):486-489.). Benzodiazepines exclusively potentiate GABA-A receptors that contain a gamma subunit, which are primarily localized at synapses. In contrast, NASs bind to alpha and beta subunits, which are present in a larger proportion of GABA-A receptors, resulting broad activity at both synaptic and extrasynaptic sites. This differentiating pharmacology supports the utility of NAS for indications where benzodiazepines have not exhibited significant utility, such as MDD and perimenopause symptoms. Perimenopause brings on a number of endocrine changes. Menstrual cycle changes typically commence with irregularities in cycle length and progress to menses cessation. The endocrine environment is highly erratic during perimenopause with fluctuations in hormonal concentrations, including follicle-stimulating hormone (FSH), estradiol, and progesterone. Without being bound by any theory, it is hypothesized that estradiol and progesterone fluctuations (which in turn (1) provides fluctuations in endogenous neurosteroids (i.e., allopregnanolone (“ALLO”), a positive allosteric modulator of GABA-A receptors) and (2) reduces GABA-A receptor activity) are in part responsible for the mood symptoms of perimenopause (Gordon, J., et al. Ovarian Hormone Fluctuation, Neurosteroids, and HPA Axis Dysregulation in Perimenopausal Depression: A Novel Heuristic Model, Am. J. Psychiatry, 172:3, March 2015, 227-236.).

In one aspect, the present invention provides a method of treating the symptoms of perimenopause or menopause comprising administering an effective amount of a GABA-A receptor PAM to a patient in need of such treatment. In some embodiments, the GABA-A receptor PAM is Compound 1 or a pharmaceutically acceptable salt thereof.

In some embodiments, the present invention provides a method of treating the vasomotor symptoms of perimenopause or menopause. In some embodiments, the vasomotor symptoms are selected from hot flushes and night sweats.

In some embodiments, the present invention provides a method of treating the sleep symptoms of perimenopause or menopause. In some embodiments, the sleep symptoms are selected from sleep disturbance and insomnia.

In some embodiments, the present invention provides a method of treating the cognitive symptoms of perimenopause or menopause. In some embodiments, the cognitive symptoms are selected from memory loss and difficulty concentrating.

In some embodiments, the present invention provides a method of treating the mood symptoms of perimenopause or menopause. In some embodiments, the mood symptoms are selected from perimenopausal depression, irritability and anxiety.

In some embodiments, the present invention provides a method of treating the sexual symptoms of perimenopause or menopause. In some embodiments, the sexual symptoms are selected from reduced libido and vaginal dryness.

In accordance with some embodiments of the present invention, at least about 15 mg, 30 mg, or 60 mg of Compound 1 or a pharmaceutically acceptable salt thereof is administered.

### GABA-A Receptor Positive Allosteric Modulators

According to the present disclosure, one or more GABA-A Receptor Positive Allosteric Modulators (GABA-A PAMs) are administered for the treatment of the symptoms of perimenopause or menopause.

GABA-A PAMs are known to those skilled in the art. The following disclosure provides guidance for the selection of GABA-A PAMs as well as non-limiting examples of GABA-A PAMs that are suitable for use in the compositions and methods of the present disclosure.

GABA-A PAMs as employed in the present methods can form a part of a pharmaceutical composition by combining a GABA-A PAM, or a pharmaceutically acceptable salt thereof, with a pharmaceutically acceptable carrier. Additionally, the compositions can include an additive selected from the group consisting of adjuvants, excipients, diluents, release-modifying agents and stabilizers. The composition can be an immediate release formulation, a delayed release formulation, a sustained release formulation or an extended release formulation.

A suitable GABA-A PAM for use in the compositions, kits and methods of the present disclosure is a compound that: is a potent (<3 μM) modulator of synaptic and/or extrasynaptic GABA-A receptors and prevents and/or reverses a depression and/or anxiety and/or sleep disturbance and/or vasomotor symptoms (i.e., hot flushes) and/or cognition and/or irritability phenotype in pre-clinical models of depression.

In some embodiments, the GABA-A PAM is selected from the group consisting of benzodiazepines, neuroactive steroids, PF-06372865 (7-ethyl-4-[3-(4-ethylsulfonyl-2-methoxyphenyl)-4-fluorophenyl]imidazo[4,5-c]pyridazine), gaboxadol and etifoxine. In some embodiments, the GABA-A PAM is etifoxine.

In some embodiments, the GABA-A PAM is a benzodiazepine. In some embodiments, the benzodiazepine is selected from the group consisting of midazolam, diazepam, chlordiazepoxide, alprazolam and adinazolam.

In some embodiments, the GABA-A PAM is a neuroactive steroid. In some embodiments, the neuroactive steroid is selected from the group consisting of pregnanolone, allopregnanolone, allotetrahydrodeoxycorticosterone, ganaxolone, alphaxolone, alphadolone, hydroxydione, minaxolone, Althesin, Renanolone, SAGE-324 (Zuranolone), SAGE-217 (3α-hydroxy-3β-methyl-21-(4-cyano-1H-pyrazol-1′-yl)-19-nor-50-pregnan-20-one), and any neuroactive steroid as described in U.S. Publication No. 2017/0240589, which is hereby incorporated by reference in its entirety.

In some embodiments, the GABA-A PAM is 3α-Hydroxy-3β-methoxymethyl-21-(1′-imidazolyl)-5α-pregnan-20-one (Compound 1) or a pharmaceutically acceptable salt thereof.

The structural formula of Compound 1 appears below.

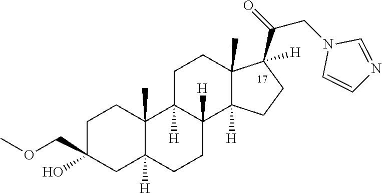

Compound 1 is a neuroactive steroid GABA-A PAM with high potency similar to clinical stage neuroactive steroids (allopregnanolone, ganaxolone, SAGE-217, alphaxolone).

The synthesis of Compound 1 is described in U.S. Publication Nos. 2004/034002 and 2009/0118248; crystalline polymorph of Compound 1 free base is described in U.S. Publication No. 2006/0074059; pharmaceutical compositions containing Compound 1 are described in U.S. Publication No. 2009/0131383, which are hereby incorporated by reference in their entirety for all purposes.

In some embodiments, the GABA-A PAM used in the formulations and methods of the present disclosure is Compound 1 or a pharmaceutically acceptable salt thereof. Salts of Compound 1 and polymorphs thereof are described in U.S. Pat. No. 10,562,930, which is hereby incorporated by reference in its entirety. In some embodiments, the pharmaceutically acceptable salt of Compound 1 used in the formulations and methods of the present disclosure is selected from the group consisting of hydrobromide, citrate, malate, mesylate, phosphate, tartrate, hydrochloride, tosylate, glucuronate, ethanesulfonate, fumarate, sulfate, napthalene-2-sulfonate, ascorbate, oxalate, napthalene-1,5-disulfonate, malonate, aminosalicylate, benzenesulfonate, isethionate, genistate, 1-hydroxy-2-napthoate, dichloroacetate, cyclamate, and ethane-1,2-disulfonate salts. In some embodiments, the salt of Compound 1 is Compound 1 Hydrobromide. In some embodiments, the salt of Compound 1 is Compound 1 Citrate. In some embodiments, the salt of Compound 1 is Compound 1 L-Malate. In some embodiments, the salt of Compound 1 is Compound 1 Mesylate. In some embodiments, the salt of Compound 1 is Compound 1 Phosphate. In some embodiments, the salt of Compound 1 is Compound 1 L(+)-Tartrate. In some embodiments, the salt of Compound 1 is Compound 1 Hydrochloride. In some embodiments, the salt of Compound 1 is Compound 1 Tosylate. In some embodiments, the salt of Compound 1 is Compound 1 Glucuronate. In some embodiments, the salt of Compound 1 is Compound 1 Ethanesulfonate.

### Formulations

The methods of the present invention can employ various formulations for administration to patients, e.g., humans in unit dosage forms, such as tablets, capsules, pills, powders, granules, sterile parenteral solutions or suspensions (e.g., intramuscular (IM), subcutaneous (SC) and intravenous (IV)), transdermal patches, and oral solutions or suspensions, and oil-water emulsions containing suitable quantities of a GABA-A PAM (e.g., Compound 1 or a pharmaceutically acceptable salt thereof).

Oral pharmaceutical dosage forms can be either solid or liquid. The solid dosage forms can be tablets, capsules, granules, films (e.g., buccal films) and bulk powders. Types of oral tablets include compressed, chewable lozenges and tablets, which can be enteric-coated, sugar-coated or film-coated. Capsules can be hard or soft gelatin capsules, while granules and powders can be provided in non-effervescent or effervescent form with the combination of other ingredients known to those skilled in the art. In some embodiments, the present oral dosage forms may include orally disintegrating tablets.

Pharmaceutically acceptable carriers utilized in tablets include binders, lubricants, diluents, disintegrating agents, coloring agents, flavoring agents, and wetting agents.

Liquid oral dosage forms include aqueous solutions, emulsions, suspensions, solutions and/or suspensions reconstituted from non-effervescent granules and effervescent preparations reconstituted from effervescent granules.

Aqueous solutions include, for example, elixirs and syrups. Emulsions can be either oil-in water or water-in-oil. Elixirs are clear, sweetened, hydroalcoholic preparations. Pharmaceutically acceptable carriers used in elixirs include solvents. Syrups can be concentrated aqueous solutions of a sugar, for example, sucrose, and can contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions can use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substance used in effervescent granules, to be reconstituted into a liquid oral dosage form, can include organic acids and a source of carbon dioxide. Coloring and flavoring agents can be used in all of the above dosage forms.

In some embodiments, the present disclosure provides a pharmaceutical composition comprising a salt of Compound 1. In some embodiments, the salt of Compound 1 is Compound 1 Hydrobromide, Compound 1 Citrate, Compound 1 L-Malate, Compound 1 Mesylate, Compound 1 Phosphate, Compound 1 L(+)-Tartrate, Compound 1 Hydrochloride, Compound 1 Tosylate, Compound 1 Glucuronate, or Compound 1 Ethanesulfonate.

### Co-Therapy

While the compositions can be administered as the sole active pharmaceutical ingredient (e.g., Compound 1) in the methods described herein, in other embodiments they can also be used in combination with one or more ingredients which are known to be therapeutically effective against one or more symptoms of perimenopause or menopause and/or compliment the therapeutic effect of the GABA-A PAM ingredient.

For example, in some embodiments, the present methods can employ a GABA-A PAM in conjunction with one or more additional anti-antidepressants agents.

In some embodiments, the GABA-A PAM is administered in combination with an additional anti-depressant agent, e.g., co-formulated or administered separately. In some embodiments, the GABA-A PAM is administered in conjunction with one or more selective serotonin reuptake inhibitors (e.g. fluoxetine), serotonin norepinephrine reuptake inhibitors (e.g. duloxetine), tricyclic antidepressants (e.g. imipramine), monoamine oxidase inhibitors (e.g. tranylcypromine), atypical monoaminergic antidepressants (e.g. mirtazapine, bupropion), serotonin modulator and stimulators (e.g., vilazodone or vortioxetine), mood stabilizers (e.g. lamotrigine), atypical antipsychotics (e.g. aripiprazole), NMDA antagonists (e.g. ketamine, derived either from r- s- or racemic ketamine), antiepileptic drugs (e.g. valproate) or combinations thereof. In some embodiments, the antiepileptic drug is selected from the group consisting of lamotrigine, valproate, and carbamazepine and oxcarbazepine. In some embodiments, the GABA-A PAM is administered in conjunction with one or more selective serotonin reuptake inhibitors, serotonin norepinephrine reuptake inhibitors, tricyclic antidepressants, monoamine oxidase inhibitors, mirtazapine, bupropion, lamotrigine, atypical antipsychotics, ketamine, esketamine, antiepileptic drugs or combinations thereof. In some embodiments, the antiepileptic drugs is selected from the group consisting of lamotrigine, valproic acid, and carbamazepine and oxcarbazepine.

In some embodiments, the GABA-A PAM is administered in combination with electroconvulsive therapy (ECT). In some embodiments, the GABA-A PAM is administered in combination with transcranial magnetic stimulation (TMS).

In some embodiments, the GABA-A PAM is administered in conjunction with one or more selective serotonin reuptake inhibitors. In some embodiments, the one or more selective serotonin reuptake inhibitors is selected from the group consisting of fluoxetine, escitalopram, citalopram, sertraline, and paroxetine. In particular embodiments, the GABA-A PAM is administered in conjunction with paroxetine.

In some embodiments, the GABA-A PAM is administered in conjunction with one or more serotonin modulator and stimulators. In some embodiments, the one or more serotonin modulator and stimulators is selected from the group consisting of vilazodone or vortioxetine.

In some embodiments, the GABA-A PAM is administered in conjunction with one or more serotonin norepinephrine reuptake inhibitors. In some embodiments, the one or more serotonin norepinephrine reuptake inhibitors is selected from the group consisting of venlafaxine, desvenlafaxine, and duloxetine.

In some embodiments, the GABA-A PAM is administered in conjunction with one or more tricyclic antidepressants. In some embodiments, the one or more tricyclic antidepressants is selected from the group consisting of amitriptyline, imipramine, and nortriptyline.

In some embodiments, the GABA-A PAM is administered in conjunction with one or more monoamine oxidase inhibitors. In some embodiments, the one or more monoamine oxidase inhibitors is selected from the group consisting of phenelzine and tranylcypromine.

In some embodiments, the GABA-A PAM is administered in conjunction with one or more atypical antipsychotics. In some embodiments, the one or more atypical antipsychotics is selected from the group consisting of lurasidone, aripiprazole, brexpiprazole, risperidone, olanzapine, quetiapine, ziprasidone, clozapine, iloperidone, paliperidone, asenapine and olanzapine/fluoxetine.

In some embodiments, the GABA-A PAM is administered in conjunction with one or more hormone replacement therapies. In some embodiments, the hormone replacement therapy is selected from an estrogen and a progestin, or a combination thereof. In some embodiments, the estrogen is selected from the group consisting of estradiol, synthetic conjugated estrogens, estradiol valerate, estradiol acetate, esterified estrogen, and estropipate. In some embodiments, the progestin is selected from the group consisting of progesterone and medroxyprogesterone acetate. In some embodiments, the combination of estrogen and progestin are selected from the group consisting of estradiol/norethindrone acetate, estradiol/drospirenone, estradiol/levonrgestrel, estradiol/norethindrone acetate, norethindrone acetate/ethinyl estradiol, estradiol/norgestimate, and conjugated estrogen/medroxyprogesterone. In some embodiments, the GABA-A PAM is administered in conjunction with tibolone.

In some embodiments, the GABA-A PAM is administered in conjunction with one or more selective estrogen receptor modulator (SERM). In some embodiments, the selective estrogen receptor modulator (SERM) is selected from the group consisting of ospemifene, and conjugated estrogens/bazedoxifene.

### Dosing

The invention provides methods for treating the symptoms of perimenopause or menopause by administering an effective amount of a GABA-A PAM (such as Compound 1 or a pharmaceutically acceptable salt thereof) to a patient in need thereof. An effective amount is an amount sufficient to eliminate or significantly reduce one or more symptoms of perimenopause or menopause or to alleviate those symptoms (e.g., reduce the symptoms, such as perimenopausal depression, anxiety or hot flushes, sleep disturbance, cognition symptoms, compared to the symptoms present prior to treatment).

According to some embodiments of the present invention, administering a GABA-A receptor PAM provides statistically significant therapeutic effect. In one embodiment, the statistically significant therapeutic effect is determined based on one or more standards or criteria provided by one or more regulatory agencies in the United States, e.g., FDA or other countries (such as Australia). In another embodiment, the statistically significant therapeutic effect is determined based on results obtained from regulatory agency approved clinical trial set up and/or procedure.

In some embodiments, the statistically significant therapeutic effect is determined based on a patient population of at least 10, 20, 50, 60, 100, 200, 300, 400, 500, 600, 700, 800, 900, 1000 or 2000. In some embodiments, the statistically significant therapeutic effect is determined based on data obtained from randomized and double-blinded clinical trial set up. In some embodiments, the statistically significant therapeutic effect is determined based on data with a p value of less than or equal to about 0.05, 0.04, 0.03, 0.02 or 0.01. In some embodiments, the statistically significant therapeutic effect is determined based on data with a confidence interval greater than or equal to 95%, 96%, 97%, 98% or 99%.

In some embodiments, the statistically significant therapeutic effect is determined by a randomized double-blind clinical trial of patients treated with Compound 1 or a pharmaceutically acceptable salt thereof and optionally in combination with standard care. In some embodiments, the statistically significant therapeutic effect is determined by a randomized clinical trial and using MENO-D as primary efficacy parameter and optionally in combination with any other commonly accepted criteria for symptom assessment.

In general, statistical analysis can include any suitable method permitted by a regulatory agency, e.g., FDA in the US or Europe or any other country. In some embodiments, statistical analysis includes non-stratified analysis, log-rank analysis, e.g., from Kaplan-Meier, Jacobson-Truax, Gulliken-Lord-Novick, Edwards-Nunnally, Hageman-Arrindel and Hierarchical Linear Modeling (HLM) and Cox regression analysis.

In the embodiments described herein, reference is made to the dose of a GABA-A PAM for the treatment of the symptoms of perimenopause or menopause, where the treated symptom is not specifically recited. However, the present disclosure contemplates the disclosed doses for the treatment of each of the symptoms of perimenopause or menopause that are described herein (e.g., vasomotor symptoms: hot flushes and night sweats; sexual symptoms: reduced libido and vaginal dryness; cognitive symptoms: memory loss and difficulty concentrating; mood symptoms: perimenopausal depression, irritability and anxiety; and sleep symptoms: sleep disturbance and insomnia).

According to the present invention, the GABA-A PAM is administered on a once or twice a day basis to provide effective relief of the symptoms of perimenopause or menopause. In some embodiments, Compound 1 or a pharmaceutically acceptable salt thereof is administered on a once or twice a day basis to provide effective relief of the symptoms of perimenopause or menopause.

In some embodiments, a total daily dose of Compound 1 is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg.

In some embodiments, the total daily dose of Compound 1 is from about 5 mg to about 120 mg, including about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, and about 120 mg, including all ranges therebetween. In some embodiments, the total daily dose of Compound 1 is from about 15 mg to about 60 mg. In some embodiments, the total daily dose of Compound 1 is from about 15 mg to about 80 mg. In some embodiments, the total daily dose of Compound 1 is from about 15 mg to about 100 mg. In some embodiments, the total daily dose of Compound 1 is from about 45 mg to about 60 mg. In some embodiments, the total daily dose of Compound 1 is from about 45 mg to about 80 mg.

In some embodiments, the total daily dose of Compound 1 is at least about 5 mg a day for the treatment of the symptoms of perimenopause or menopause. In some embodiments, the total daily dose of Compound 1 is at least about 10 mg a day for the treatment of the symptoms of perimenopause or menopause. In some embodiments, the total daily dose of Compound 1 is at least about 15 mg a day for the treatment of the symptoms of perimenopause or menopause. In some embodiments, the total daily dose of Compound 1 is at least about 20 mg a day for the treatment of the symptoms of perimenopause or menopause. In some embodiments, the total daily dose of Compound 1 is at least about 25 mg a day for the treatment of the symptoms of perimenopause or menopause. In some embodiments, the total daily dose of Compound 1 is at least about 30 mg a day for the treatment of the symptoms of perimenopause or menopause. In some embodiments, the total daily dose of Compound 1 is at least about 35 mg a day for the treatment of the symptoms of perimenopause or menopause. In some embodiments, the total daily dose of Compound 1 is at least about 40 mg a day for the treatment of the symptoms of perimenopause or menopause. In some embodiments, the total daily dose of Compound 1 is at least about 45 mg a day for the treatment of the symptoms of perimenopause or menopause. In some embodiments, the total daily dose of Compound 1 is at least about 50 mg a day for the treatment of the symptoms of perimenopause or menopause. In some embodiments, the total daily dose of Compound 1 is at least about 55 mg a day for the treatment of the symptoms of perimenopause or menopause. In some embodiments, the total daily dose of Compound 1 is at least about 60 mg a day for the treatment of the symptoms of perimenopause or menopause. In some embodiments, the total daily dose of Compound 1 is at least about 65 mg a day for the treatment of the symptoms of perimenopause or menopause. In some embodiments, the total daily dose of Compound 1 is at least about 70 mg a day for the treatment of the symptoms of perimenopause or menopause. In some embodiments, the total daily dose of Compound 1 is at least about 75 mg a day for the treatment of the symptoms of perimenopause or menopause. In some embodiments, the total daily dose of Compound 1 is at least about 80 mg a day for the treatment of the symptoms of perimenopause or menopause. In some embodiments, the total daily dose of Compound 1 is at least about 85 mg a day for the treatment of the symptoms of perimenopause or menopause. In some embodiments, the total daily dose of Compound 1 is at least about 90 mg a day for the treatment of the symptoms of perimenopause or menopause. In some embodiments, the total daily dose of Compound 1 is at least about 95 mg a day for the treatment of the symptoms of perimenopause or menopause. In some embodiments, the total daily dose of Compound 1 is at least about 100 mg a day for the treatment of the symptoms of perimenopause or menopause. In some embodiments, the total daily dose of Compound 1 is at least about 105 mg a day for the treatment of the symptoms of perimenopause or menopause. In some embodiments, the total daily dose of Compound 1 is at least about 110 mg a day for the treatment of the symptoms of perimenopause or menopause. In some embodiments, the total daily dose of Compound 1 is at least about 115 mg a day for the treatment of the symptoms of perimenopause or menopause. In some embodiments, the total daily dose of Compound 1 is at least about 120 mg a day for the treatment of the symptoms of perimenopause or menopause.

In some embodiments, the total daily dose of Compound 1 is about 5 mg a day for the treatment of the symptoms of perimenopause or menopause. In some embodiments, the total daily dose of Compound 1 is about 10 mg a day for the treatment of the symptoms of perimenopause or menopause. In some embodiments, the total daily dose of Compound 1 is about 15 mg a day for the treatment of the symptoms of perimenopause or menopause. In some embodiments, the total daily dose of Compound 1 is about 20 mg a day for the treatment of the symptoms of perimenopause or menopause. In some embodiments, the total daily dose of Compound 1 is about 25 mg a day for the treatment of the symptoms of perimenopause or menopause. In some embodiments, the total daily dose of Compound 1 is about 30 mg a day for the treatment of the symptoms of perimenopause or menopause. In some embodiments, the total daily dose of Compound 1 is about 35 mg a day for the treatment of the symptoms of perimenopause or menopause. In some embodiments, the total daily dose of Compound 1 is about 40 mg a day for the treatment of the symptoms of perimenopause or menopause. In some embodiments, the total daily dose of Compound 1 is about 45 mg a day for the treatment of the symptoms of perimenopause or menopause. In some embodiments, the total daily dose of Compound 1 is about 50 mg a day for the treatment of the symptoms of perimenopause or menopause. In some embodiments, the total daily dose of Compound 1 is about 55 mg a day for the treatment of the symptoms of perimenopause or menopause. In some embodiments, the total daily dose of Compound 1 is about 60 mg a day for the treatment of the symptoms of perimenopause or menopause. In some embodiments, the total daily dose of Compound 1 is about 65 mg a day for the treatment of the symptoms of perimenopause or menopause. In some embodiments, the total daily dose of Compound 1 is about 70 mg a day for the treatment of the symptoms of perimenopause or menopause. In some embodiments, the total daily dose of Compound 1 is about 75 mg a day for the treatment of the symptoms of perimenopause or menopause. In some embodiments, the total daily dose of Compound 1 is about 80 mg a day for the treatment of the symptoms of perimenopause or menopause. In some embodiments, the total daily dose of Compound 1 is about 85 mg a day for the treatment of the symptoms of perimenopause or menopause. In some embodiments, the total daily dose of Compound 1 is about 90 mg a day for the treatment of the symptoms of perimenopause or menopause. In some embodiments, the total daily dose of Compound 1 is about 95 mg a day for the treatment of the symptoms of perimenopause or menopause. In some embodiments, the total daily dose of Compound 1 is about 100 mg a day for the treatment of the symptoms of perimenopause or menopause. In some embodiments, the total daily dose of Compound 1 is about 105 mg a day for the treatment of the symptoms of perimenopause or menopause. In some embodiments, the total daily dose of Compound 1 is about 110 mg a day for the treatment of the symptoms of perimenopause or menopause. In some embodiments, the total daily dose of Compound 1 is about 115 mg a day for the treatment of the symptoms of perimenopause or menopause. In some embodiments, the total daily dose of Compound 1 is about 120 mg a day for the treatment of the symptoms of perimenopause or menopause.

In some embodiments, about 5 mg of Compound 1 once a day is selected to provide a substantial reduction in the symptoms of perimenopause or menopause. In some embodiments, about 5 mg of Compound 1 twice a day is selected to provide a substantial reduction in the symptoms of perimenopause or menopause. In some embodiments, about 10 mg of Compound 1 once a day is selected to provide a substantial reduction in the symptoms of perimenopause or menopause. In some embodiments, about 10 mg of Compound 1 twice a day is selected to provide a substantial reduction in the symptoms of perimenopause or menopause. In some embodiments, about 15 mg of Compound 1 once a day is selected to provide a substantial reduction in the symptoms of perimenopause or menopause. In some embodiments, about 15 mg of Compound 1 twice a day is selected to provide a substantial reduction in the symptoms of perimenopause or menopause. In some embodiments, about 20 mg of Compound 1 once a day is selected to provide a substantial reduction in the symptoms of perimenopause or menopause. In some embodiments, about 20 mg of Compound 1 twice a day is selected to provide a substantial reduction in the symptoms of perimenopause or menopause. In some embodiments, about 25 mg of Compound 1 once a day is selected to provide a substantial reduction in the symptoms of perimenopause or menopause. In some embodiments, about 25 mg of Compound 1 twice a day is selected to provide a substantial reduction in the symptoms of perimenopause or menopause. In some embodiments, about 30 mg of Compound 1 once a day is selected to provide a substantial reduction in the symptoms of perimenopause or menopause. In some embodiments, about 30 mg of Compound 1 twice a day is selected to provide a substantial reduction in the symptoms of perimenopause or menopause. In some embodiments, about 30 mg of Compound 1 once a day is selected to provide a substantial reduction in the symptoms of perimenopause or menopause. In some embodiments, about 30 mg of Compound 1 twice a day is selected to provide a substantial reduction in the symptoms of perimenopause or menopause. In some embodiments, about 35 mg of Compound 1 once a day is selected to provide a substantial reduction in the symptoms of perimenopause or menopause. In some embodiments, about 35 mg of Compound 1 twice a day is selected to provide a substantial reduction in the symptoms of perimenopause or menopause. In some embodiments, about 40 mg of Compound 1 once a day is selected to provide a substantial reduction in the symptoms of perimenopause or menopause. In some embodiments, about 40 mg of Compound 1 twice a day is selected to provide a substantial reduction in the symptoms of perimenopause or menopause. In some embodiments, about 45 mg of Compound 1 once a day is selected to provide a substantial reduction in the symptoms of perimenopause or menopause. In some embodiments, about 45 mg of Compound 1 twice a day is selected to provide a substantial reduction in the symptoms of perimenopause or menopause. In some embodiments, about 50 mg of Compound 1 once a day is selected to provide a substantial reduction in the symptoms of perimenopause or menopause. In some embodiments, about 50 mg of Compound 1 twice a day is selected to provide a substantial reduction in the symptoms of perimenopause or menopause. In some embodiments, about 55 mg of Compound 1 once a day is selected to provide a substantial reduction in the symptoms of perimenopause or menopause. In some embodiments, about 55 mg of Compound 1 twice a day is selected to provide a substantial reduction in the symptoms of perimenopause or menopause. In some embodiments, about 60 mg of Compound 1 once a day is selected to provide a substantial reduction in the symptoms of perimenopause or menopause. In some embodiments, about 60 mg of Compound 1 twice a day is selected to provide a substantial reduction in the symptoms of perimenopause or menopause. In some embodiments, about 65 mg of Compound 1 once a day is selected to provide a substantial reduction in the symptoms of perimenopause or menopause. In some embodiments, about 70 mg of Compound 1 once a day is selected to provide a substantial reduction in the symptoms of perimenopause or menopause. In some embodiments, about 75 mg of Compound 1 once a day is selected to provide a substantial reduction in the symptoms of perimenopause or menopause. In some embodiments, about 80 mg of Compound 1 once a day is selected to provide a substantial reduction in the symptoms of perimenopause or menopause. In some embodiments, about 85 mg of Compound 1 once a day is selected to provide a substantial reduction in the symptoms of perimenopause or menopause. In some embodiments, about 90 mg of Compound 1 once a day is selected to provide a substantial reduction in the symptoms of perimenopause or menopause. In some embodiments, about 95 mg of Compound 1 once a day is selected to provide a substantial reduction in the symptoms of perimenopause or menopause. In some embodiments, about 100 mg of Compound 1 once a day is selected to provide a substantial reduction in the symptoms of perimenopause or menopause. In some embodiments, about 105 mg of Compound 1 once a day is selected to provide a substantial reduction in the symptoms of perimenopause or menopause. In some embodiments, about 110 mg of Compound 1 once a day is selected to provide a substantial reduction in the symptoms of perimenopause or menopause. In some embodiments, about 115 mg of Compound 1 once a day is selected to provide a substantial reduction in the symptoms of perimenopause or menopause. In some embodiments, about 120 mg of Compound 1 once a day is selected to provide a substantial reduction in the symptoms of perimenopause or menopause.

Reduction of the symptoms of perimenopause or menopause can be determined by various methods. The method used to determine the reduction of the symptom will depend on the type of symptom that is assessed. For example, in some embodiments, the treated symptom of perimenopause or menopause is a mood symptom and the method should be appropriate for determining the treatment of mood symptoms. Various methods for determining the reduction of the mood symptoms, sexual symptoms, sleep symptoms and cognitive symptoms of perimenopause or menopause are describe herein; however, any suitable method known to those skilled in the art may be used to determine the symptom treatment according to the presently disclosed methods.

In some embodiments, the effectiveness of a dosage regimen for treating the symptoms of perimenopause or menopause can be determined by evaluation via MENO-D. In some embodiments, the effectiveness of a dosage regimen for treating the mood symptoms of perimenopause or menopause can be determined by evaluation via Hamilton Depression Rating Scale (HAM-D). In some embodiments, the effectiveness of a dosage regimen for treating the mood symptoms of perimenopause or menopause can be determined by evaluation via a Montgomery Asberg Depression Rating Scale (MADRS). In yet some other embodiments, the effectiveness of a dosage regimen for treating the mood symptoms of perimenopause or menopause can be determined by evaluation via HAM-D, MADRS, Hamilton Rating Scale for anxiety (HAM-A), Clinical Global Impression (CGI) subscale scores (i.e., Severity of Illness Subscale (CGI-S), Global Improvement Subscale (CGI-I), or Efficacy Index Subscale), Symptoms of Depression Questionnaire (SDQ), Pittsburgh Sleep Quality Index (PSQI), or any combination thereof. In some embodiments, the effectiveness of a dosage regimen for treating the mood symptoms of perimenopause or menopause can be determined by evaluation of GAD-7.

According to some embodiments of the present invention, the dosing frequency and dose amount per administration of Compound 1 are selected to provide therapeutic effects for the treatment of the symptoms of perimenopause or menopause that is refractory to other treatments.

According to some embodiments of the present invention, the dosing frequency and dose amount per administration of the GABA-A PAM are selected to provide therapeutic effects for the treatment of the symptoms of perimenopause or menopause that is partially responsive to other therapies. According to some embodiments of the present invention, the dosing frequency and dose amount per administration of the GABA-A PAM are selected to provide an adjunctive treatment for treating the symptoms of perimenopause or menopause. According to some embodiments of the present invention, the dosing frequency and dose amount per administration of the GABA-A PAM are selected to provide therapeutic effects for the treatment of the symptoms of perimenopause or menopause that is partially responsive to treatment with an antidepressant agent. According to some embodiments of the present invention, the dosing frequency and dose amount per administration of the GABA-A PAM are selected to provide therapeutic effects for the treatment of the symptoms of perimenopause or menopause that is partially responsive to treatment with hormone replacement therapy. According to some embodiments of the present invention, the dosing frequency and dose amount per administration of the GABA-A PAM are selected to provide therapeutic effects for the treatment of the symptoms of perimenopause or menopause that is partially responsive to treatment with selective estrogen receptor modulator (SERM).

In some embodiments, the dosing frequency and dose amount per administration of Compound 1 are selected to provide therapeutic effects for the acute symptoms of perimenopause or menopause. In some embodiments, the dosing frequency and dose amount per administration of Compound 1 are selected to provide therapeutic effects for the treatment of the symptoms of perimenopause or menopause and, after the acute treatment of the symptoms, Compound 1 is no longer administered and a dosing frequency and dose amount of second therapeutic agent is selected to provide therapeutic effects for the chronic treatment of the symptoms.

In some embodiments, the dosing frequency and dose amount per administration of the GABA-A PAM are selected to provide therapeutic effects for the acute treatment of the symptoms of perimenopause or menopause and, after the acute treatment of the symptoms, a dosing frequency and dose amount of the GABA-A PAM is selected to provide therapeutic effects for the chronic treatment of the symptoms. In some embodiments, the daily dosing of the GABA-A PAM for the acute treatment of the symptoms of perimenopause or menopause is greater than the daily dosing of the GABA-A PAM for the chronic treatment of the symptoms.

In some embodiments, the dosing frequency and dose amount per administration of the GABA-A PAM are selected to prevent the recurrence of the symptoms of perimenopause or menopause. In some embodiments, the dosing frequency and dose amount per administration of the GABA-A PAM are selected to maintain remission of the symptoms of perimenopause or menopause.

In some embodiments, the GABA-A PAM (e.g., Compound 1 or a pharmaceutically acceptable salt thereof) is administered on a once a day or twice a day basis for at least a week, for example, about a week, about 2 weeks, about 3 weeks, about 4 weeks, about 5 weeks, about 6 weeks, about 7 weeks, about 8 weeks, about 9 weeks, about 10 weeks, about 12 weeks, about 18 weeks, about 24 weeks, and about 50 weeks.

In some embodiments, at least about 5 mg or about 5 mg of Compound 1 or a pharmaceutically acceptable salt thereof is administered on a once a day or twice a day basis for at least a week. In some embodiments, at least about 10 mg or about 10 mg of Compound 1 or a pharmaceutically acceptable salt thereof is administered on a once a day or twice a day basis for at least a week. In some embodiments, at least about 15 mg or about 15 mg of Compound 1 or a pharmaceutically acceptable salt thereof is administered on a once a day or twice a day basis for at least a week. In some embodiments, at least about 20 mg or about 20 mg of Compound 1 or a pharmaceutically acceptable salt thereof is administered on a once a day or twice a day basis for at least a week. In some embodiments, at least about 25 mg or about 25 mg of Compound 1 or a pharmaceutically acceptable salt thereof is administered on a once a day or twice a day basis for at least a week. In some embodiments, at least about 30 mg or about 30 mg of Compound 1 or a pharmaceutically acceptable salt thereof is administered on a once a day or twice a day basis for at least a week. In some embodiments, at least about 35 mg or about 35 mg of Compound 1 or a pharmaceutically acceptable salt thereof is administered on a once a day or twice a day basis for at least a week. In some embodiments, at least about 40 mg or about 40 mg of Compound 1 or a pharmaceutically acceptable salt thereof is administered on a once a day or twice a day basis for at least a week. In some embodiments, at least about 45 mg or about 45 mg of Compound 1 or a pharmaceutically acceptable salt thereof is administered on a once a day or twice a day basis for at least a week. In some embodiments, at least about 50 mg or about 50 mg of Compound 1 or a pharmaceutically acceptable salt thereof is administered on a once a day or twice a day basis for at least a week. In some embodiments, at least about 55 mg or about 55 mg of Compound 1 or a pharmaceutically acceptable salt thereof is administered on a once a day or twice a day basis for at least a week. In some embodiments, at least about 60 mg or about 60 mg of Compound 1 or a pharmaceutically acceptable salt thereof is administered on a once a day or twice a day basis for at least a week. In some embodiments, at least about 65 mg or about 65 mg of Compound 1 or a pharmaceutically acceptable salt thereof is administered on a once a day basis for at least a week. In some embodiments, at least about 70 mg or about 70 mg of Compound 1 or a pharmaceutically acceptable salt thereof is administered on a once a day basis for at least a week. In some embodiments, at least about 75 mg or about 75 mg of Compound 1 or a pharmaceutically acceptable salt thereof is administered on a once a day basis for at least a week. In some embodiments, at least about 80 mg or about 80 mg of Compound 1 or a pharmaceutically acceptable salt thereof is administered on a once a day basis for at least a week. In some embodiments, at least about 85 mg or about 85 mg of Compound 1 or a pharmaceutically acceptable salt thereof is administered on a once a day basis for at least a week. In some embodiments, at least about 90 mg or about 90 mg of Compound 1 or a pharmaceutically acceptable salt thereof is administered on a once a day basis for at least a week. In some embodiments, at least about 95 mg or about 95 mg of Compound 1 or a pharmaceutically acceptable salt thereof is administered on a once a day basis for at least a week. In some embodiments, at least about 100 mg or about 100 mg of Compound 1 or a pharmaceutically acceptable salt thereof is administered on a once a day basis for at least a week. In some embodiments, at least about 105 mg or about 105 mg of Compound 1 or a pharmaceutically acceptable salt thereof is administered on a once a day basis for at least a week. In some embodiments, at least about 110 mg or about 110 mg of Compound 1 or a pharmaceutically acceptable salt thereof is administered on a once a day basis for at least a week. In some embodiments, at least about 115 mg or about 115 mg of Compound 1 or a pharmaceutically acceptable salt thereof is administered on a once a day basis for at least a week. In some embodiments, at least about 120 mg or about 120 mg of Compound 1 or a pharmaceutically acceptable salt thereof is administered on a once a day basis for at least a week.

According to some embodiments, the substantial reduction in the symptoms of perimenopause or menopause provided by the methods of the present disclosure requires treatment for a specified time interval (e.g., at least one week) before the patient experiences substantial reduction of the symptom (i.e., there is an induction period before the patient experiences a substantial reduction in the symptoms of perimenopause or menopause). In some embodiments, after treatment for at least one week, at least two weeks, at least three weeks, at least four weeks, at least five weeks, at least six weeks, at least seven weeks or at least eight weeks, the patient experiences a substantial reduction of the symptoms of perimenopause or menopause compared to prior to the treatment. In some embodiments, after treatment for at least one week the patient experiences a substantial reduction of the symptoms of perimenopause or menopause compared to prior to the treatment. In some embodiments, after treatment for less than one week (for example, after 1 day, 2 days, 3 days, 4 days, 5 days, or days of Compound 1 administration) the patient experiences a substantial reduction of the symptoms of perimenopause or menopause compared to prior to the treatment. According to such embodiments, the substantial reduction in the symptoms of perimenopause or menopause may be expressed using any of the methods described herein (for example, decline in MENO-D compared to prior to the treatment, reduction in the daily total of hot flushes compared to prior to the treatment, etc.).

In some embodiments, the dosing frequency and dose amount per administration of the GABA-A PAM are selected to provide therapeutic effects for the treatment of the mood symptoms of perimenopause or menopause. In some embodiments, the present invention provides a method of treating the mood symptoms of perimenopause or menopause such that after the treatment the patient experiences a substantial reduction in the mood symptoms of perimenopause or menopause. In some embodiments, the mood symptoms are selected from perimenopausal depression, anxiety and irritability.

The short form of the Profile of Mood States (POMS-SF) is a questionnaire to evaluate psychological distress consisting of 37 items (Shacham, S (1983, A shortened version of the Profile of Mood States, J of Personality Assessment, 47, 305-306). The POMS-SF yields a global Total Mood Disturbance score as well as six subscores: Fatigue-Inertia, Vigor-Activity, Tension-Anxiety, Depression-Dejection, Anger-Hostility and Confusion-Bewilderment Each question has five possible answers, score from 0 to 4 points (from the “not at all” to “extremely”). The range of possible global Total Mood Disturbance score is 0 to 148.

In some embodiments, after the treatment the patient experiences a substantial reduction in the mood symptoms of perimenopause or menopause that is characterized by at least about a 20% decline in total POMS-SF value compared to prior to the treatment. In some embodiments, the reduction in the mood symptoms of perimenopause or menopause is characterized by a decline in POMS-SF value ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction in the mood symptoms of perimenopause or menopause that is characterized by at least a one point decline in POMS-SF value compared to prior to the treatment. In some embodiments, the reduction in the mood symptoms of perimenopause or menopause is characterized by a decline in POMS-SF value ranging from about one point to about ten points, for example, about one point, about two points, about three points, about four points, about five points, about six points, about seven points, about eight points, about nine points and about ten points compared to prior to the treatment. In some embodiments, the reduction in the mood symptoms of perimenopause or menopause is characterized by a decline POMS-SF value of about two points. In some embodiments, the reduction in the mood symptoms of perimenopause or menopause is characterized by a decline in POMS-SF value of about three points. In some embodiments, the reduction in the mood symptoms of perimenopause or menopause is characterized by a decline in POMS-SF value of about four points. In some embodiments, the reduction in the mood symptoms of perimenopause or menopause is characterized by a decline in POMS-SF value of about five points. In some embodiments, the reduction in the mood symptoms of perimenopause or menopause is characterized by a decline POMS-SF value of about six points. In some embodiments, the reduction in the mood symptoms of perimenopause or menopause is characterized by a decline in POMS-SF value of about seven points. In some embodiments, the reduction in the mood symptoms of perimenopause or menopause is characterized by a decline in POMS-SF value of about eight points. In some embodiments, the in the mood symptoms of perimenopause or menopause is characterized by a decline in POMS-SF value of about nine points. In some embodiments, the reduction in the mood symptoms of perimenopause or menopause is characterized by a decline in POMS-SF value of about ten points.

In some embodiments, after the treatment the patient experiences a substantial reduction in the mood symptoms of perimenopause or menopause of that is characterized by at least about a 10% decline in at least one POMS-SF subscale value selected from Fatigue-Inertia, Vigor-Activity, Tension-Anxiety, Depression-Dejection, Anger-Hostility and Confusion-Bewilderment compared to prior to the treatment. In some embodiments, the reduction in the mood symptoms of perimenopause or menopause is characterized by a decline in at least one subscale value selected from Fatigue-Inertia, Vigor-Activity, Tension-Anxiety, Depression-Dejection, Anger-Hostility and Confusion-Bewilderment value ranging from about 10% to about 100%, for example, about 20%, about 30%, about 40%, about 50%, about 60%, about 70%, about 80%, about 90%, and about 100%, compared to prior to the treatment.

The Meno-D is a perimenopausal depression rating scale consisting of 12 items, each items is scored on a 5-point scale (ranging from 0 to 4) (Kulkami J, et al., Development and validation of a new rating scale for perimenopausal depression-the Meno-D. Transl Psychiatry. 2018 Jun. 28; 8(1):123). The total score of the 12 items ranges from 0 to 60 with higher scores indicating more severe perimenopausal depression. The MENO-D has five factors (or domains): Self (four items), Sexual (two items), Somatic (two items), Cognitive (two items) and Sleep (two items). MENO-D assesses the patient's self-esteem, sense of isolation, paranoid thinking, anxiety, interest in socialising, changes in libido and sexual activity, any decrease in energy, physical pain, changes in weight, subjective changes in memory and concentration, and increases in irritability and/or sleep disturbances.

In some embodiments, after the treatment the patient experiences a substantial reduction of perimenopausal depression that is characterized by at least about a 30% decline in MENO-D value compared to prior to the treatment. In some embodiments, after the treatment the patient experiences a substantial reduction of perimenopausal depression that is characterized by at least about a 50% decline in MENO-D value compared to prior to the treatment. In some embodiments, the reduction of perimenopausal depression is characterized by a decline in MENO-D value ranging from about 30% to about 100%, for example, about 30%, about 40%, about 50%, about 60%, about 70%, about 80%, about 90%, and about 100%, compared to prior to the treatment.

In some embodiments, after the treatment the patient experiences a substantial reduction of perimenopausal depression that is characterized by at least a one point decline in MENO-D value compared to prior to the treatment. In some embodiments, the reduction of perimenopausal depression is characterized by a decline in MENO-D value ranging from about one point to about twenty points, for example, about one point, about two points, about three points, about four points, about five points, about six points, about seven points, about eight points, about nine points, about ten points, about eleven points, about twelve points, about thirteen points, about fourteen points, about fifteen points, about sixteen points, about seventeen points, about eighteen points, about nineteen points, and about twenty points compared to prior to the treatment. In some embodiments, the reduction of perimenopausal depression is characterized by a decline MENO-D value of about two points. In some embodiments, the reduction of perimenopausal depression is characterized by a decline in MENO-D value of about three points. In some embodiments, the reduction of perimenopausal depression is characterized by a decline in MENO-D value of about four points. In some embodiments, the reduction of perimenopausal depression is characterized by a decline in MENO-D value of about five points. In some embodiments, the reduction of perimenopausal depression is characterized by a decline MENO-D value of about six points. In some embodiments, the reduction of perimenopausal depression is characterized by a decline in MENO-D value of about seven points. In some embodiments, the reduction of perimenopausal depression is characterized by a decline in MENO-D value of about eight points. In some embodiments, the reduction of perimenopausal depression is characterized by a decline in MENO-D value of about nine points. In some embodiments, the reduction of perimenopausal depression is characterized by a decline MENO-D value of about ten points. In some embodiments, the reduction of perimenopausal depression is characterized by a decline in MENO-D value of about eleven points. In some embodiments, the reduction of perimenopausal depression is characterized by a decline in MENO-D value of about twelve points. In some embodiments, the reduction of perimenopausal depression is characterized by a decline in MENO-D value of about thirteen points. In some embodiments, the reduction of perimenopausal depression is characterized by a decline MENO-D value of about fourteen points. In some embodiments, the reduction of perimenopausal depression is characterized by a decline in MENO-D value of about fifteen points. In some embodiments, the reduction of perimenopausal depression is characterized by a decline in MENO-D value of about sixteen points. In some embodiments, the reduction of perimenopausal depression is characterized by a decline in MENO-D value of about seventeen points. In some embodiments, the reduction of perimenopausal depression is characterized by a decline MENO-D value of about eighteen points. In some embodiments, the reduction of perimenopausal depression is characterized by a decline in MENO-D value of about nineteen points. In some embodiments, the reduction of perimenopausal depression is characterized by a decline in MENO-D value of about twenty points.

In some embodiments, after the treatment the patient experiences a substantial reduction of perimenopausal depression that is characterized by at least 1 point decline in at least one factor value selected from Self, Sexual, Somatic, Cognitive and Sleep compared to prior to the treatment. In some embodiments, the reduction of perimenopausal depression is characterized by a decline in at least one factor value selected from Self, Sexual, Somatic, Cognitive and Sleep value ranging from 1 point to 8 points, for example, 1 point, 2 points, 3 points, 4 points, 5 points, 6 points, 7 points, and 8 points, compared to prior to the treatment.

In some embodiments, after the treatment the patient experiences a substantial reduction of perimenopausal depression that is characterized by at least about a 30% decline in at least one factor value selected from Self, Sexual, Somatic, Cognitive and Sleep compared to prior to the treatment. In some embodiments, after the treatment the patient experiences a substantial reduction of perimenopausal depression that is characterized by at least about a 50% decline in at least one factor value selected from Self, Sexual, Somatic, Cognitive and Sleep compared to prior to the treatment. In some embodiments, the reduction of perimenopausal depression is characterized by a decline in at least one factor value selected from Self, Sexual, Somatic, Cognitive and Sleep ranging from about 30% to about 100%, for example, about 30%, about 40%, about 50%, about 60%, about 70%, about 80%, about 90%, and about 100%, compared to prior to the treatment.

The HAM-D is a depression rating scale consisting of 17 items, eight items are scored on a 5-point scale (ranging from 0 to 4), and 9 items are scores on a 3-point scale (ranging from 0 to 2). The total score of the 17 items ranges from 0 to 50 with higher scores indicating greater depression. The total score the 17 items is used to categorize the severity of depression: normal (total score between 0 and 7), mild depression (total score between 8 and 13), moderate depression (total score between 14-18), severe depression (total score between 19-22). Therefore, a decrease in the total score or on individual item scores indicates improvement Hamilton, M. A Rating Scale for Depression, Journal of Neurology, Neurosurgery, and Psychiatry. (1960) 23, pages 56-62.

In some embodiments, after the treatment the patient experiences a substantial reduction of perimenopausal depression that is characterized by at least about a 30% decline in total Hamilton Depression Rating Scale (HAM-D) value compared to prior to the treatment.

In some embodiments, the reduction of perimenopausal depression is characterized by a decline in HAM-D value ranging from about 30% to about 100%, for example, about 30%, about 40%, about 50%, about 60%, about 70%, about 80%, about 90%, and about 100%, compared to prior to the treatment.

In some embodiments, after the treatment the patient experiences a substantial reduction of perimenopausal depression that is characterized by at least a one point decline in HAM-D value compared to prior to the treatment. In some embodiments, the reduction of perimenopausal depression is characterized by a decline in HAM-D value ranging from about one point to about twenty points, for example, about one point, about two points, about three points, about four points, about five points, about six points, about seven points, about eight points, about nine points, about ten points, about eleven points, about twelve points, about thirteen points, about fourteen points, about fifteen points, about sixteen points, about seventeen points, about eighteen points, about nineteen points, and about twenty points compared to prior to the treatment. In some embodiments, the reduction of perimenopausal depression is characterized by a decline HAM-D value of about two points. In some embodiments, the reduction of perimenopausal depression is characterized by a decline in HAM-D value of about three points. In some embodiments, the reduction of perimenopausal depression is characterized by a decline in HAM-D value of about four points. In some embodiments, the reduction of perimenopausal depression is characterized by a decline in HAM-D value of about five points. In some embodiments, the reduction of perimenopausal depression is characterized by a decline HAM-D value of about six points. In some embodiments, the reduction of perimenopausal depression is characterized by a decline in HAM-D value of about seven points. In some embodiments, the reduction of perimenopausal depression is characterized by a decline in HAM-D value of about eight points. In some embodiments, the reduction of perimenopausal depression is characterized by a decline in HAM-D value of about nine points. In some embodiments, the reduction of perimenopausal depression is characterized by a decline HAM-D value of about ten points. In some embodiments, the reduction of perimenopausal depression is characterized by a decline in HAM-D value of about eleven points. In some embodiments, the reduction of perimenopausal depression is characterized by a decline in HAM-D value of about twelve points. In some embodiments, the reduction of perimenopausal depression is characterized by a decline in HAM-D value of about thirteen points. In some embodiments, the reduction of perimenopausal depression is characterized by a decline HAM-D value of about fourteen points. In some embodiments, the reduction of perimenopausal depression is characterized by a decline in HAM-D value of about fifteen points. In some embodiments, the reduction of perimenopausal depression is characterized by a decline in HAM-D value of about sixteen points. In some embodiments, the reduction of perimenopausal depression is characterized by a decline in HAM-D value of about seventeen points. In some embodiments, the reduction of perimenopausal depression is characterized by a decline HAM-D value of about eighteen points. In some embodiments, the reduction of perimenopausal depression is characterized by a decline in HAM-D value of about nineteen points. In some embodiments, the reduction of perimenopausal depression is characterized by a decline in HAM-D value of about twenty points.

In some embodiments, after the treatment the patient experiences a substantial reduction of perimenopausal depression that is characterized by at least a one category change in HAM-D severity classification compared to prior to the treatment. In some embodiments, the reduction of perimenopausal depression is characterized by a one category change HAM-D severity classification compared to prior to the treatment. In some embodiments, the reduction of perimenopausal depression is characterized by a two category change HAM-D severity classification compared to prior to the treatment. In some embodiments, the reduction of perimenopausal depression is characterized by a three category change HAM-D severity classification compared to prior to the treatment. In some embodiments, the reduction of perimenopausal depression is characterized by remission according to HAM-D value after said treatment (i.e., total HAM-D value of 7 or less).

The Montgomery Asberg Depression Rating Scale (MADRS) is a depression rating scale consisting of 10 items, each rated 0 to 6. The 10 items represent the core symptoms of depressive illness. The total score of the 10 items ranges from 0 to 60. Decrease in the total score or on individual items indicates improvement (Montgomery S. A. and Asberg M. A, New Depression Scale Designed to be Sensitive to Change, Br. J. Psychiatry. (1979) April; 134, pages 382-9.).

In some embodiments, after the treatment the patient experiences a substantial reduction of perimenopausal depression that is characterized by at least about a 30% decline in Montgomery Asberg Depression Rating Scale (MADRS) compared to prior to the treatment. In some embodiments, the reduction of perimenopausal depression is characterized by a decline in MADRS value ranging from about 30% to about 100%, for example, about 30%, about 40%, about 50%, about 60%, about 70%, about 80%, about 90%, and about 100%, compared to prior to the treatment.

In some embodiments, after the treatment the patient experiences a substantial reduction of perimenopausal depression that is characterized by at least a one point decline in MADRS value compared to prior to the treatment. In some embodiments, the reduction of perimenopausal depression is characterized by a decline in MADRS value ranging from about one point to about five points, for example, about one point, about two points, about three points, about four points, and about five points compared to prior to the treatment. In some embodiments, the reduction of perimenopausal depression is characterized by a decline MADRS value of about two points. In some embodiments, the reduction of perimenopausal depression is characterized by a decline in MADRS value of about three points. In some embodiments, the reduction of perimenopausal depression is characterized by a decline in MADRS value of about four points. In some embodiments, the reduction of perimenopausal depression is characterized by a decline in MADRS value of about five points. In some embodiments, the reduction of perimenopausal depression is characterized by remission according to MADRS value after said treatment (i.e., MADRS value of 12 or less).

The Hamilton Rating Scale for Anxiety (HAM-A) is an anxiety rating scale consisting of 14 items that assess anxious mood, tension, fear, insomnia, intellectual (cognitive) functioning, depressed mood, behavior at interview, somatic (sensory), cardiovascular, respiratory, gastrointestinal, genitourinary, autonomic, and somatic (muscular) symptom (Hamilton, M. The Assessment of Anxiety States by Rating, Br J Med Psychol. (1959); 32 (1), pages 50-5). Each symptom is rated from 0 (absent) to 4 (maximum severity) scale. The total score is used to categorize the severity of anxiety: mild severity (total score less than 17), mild to moderate severity (total score between 18-24), and moderate to severe (total score between 25-30). Total scores range from 0 to 56 with higher scores indicating greater severity.

In some embodiments, after the treatment the patient experiences a substantial reduction of perimenopausal anxiety that is characterized by at least about a 30% decline in total HAM-A value compared to prior to the treatment. In some embodiments, the reduction of perimenopausal anxiety is characterized by a decline in HAM-A value ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction of perimenopausal anxiety that is characterized by at least a one point decline in HAM-A value compared to prior to the treatment. In some embodiments, the reduction of perimenopausal depression is characterized by a decline in HAM-A value ranging from about one point to about five points, for example, about one point, about two points, about three points, about four points, and about five points compared to prior to the treatment. In some embodiments, the reduction of perimenopausal depression is characterized by a decline HAM-A value of about two points. In some embodiments, the reduction of perimenopausal depression is characterized by a decline in HAM-A value of about three points. In some embodiments, the reduction of perimenopausal depression is characterized by a decline in HAM-A value of about four points. In some embodiments, the reduction of perimenopausal depression is characterized by a decline in HAM-A value of about five points.

In some embodiments, after the treatment the patient experiences a substantial reduction of perimenopausal depression that is characterized by at least a one category change in HAM-A severity classification compared to prior to the treatment. In some embodiments, the reduction of perimenopausal depression is characterized by a one category change HAM-A severity classification compared to prior to the treatment. In some embodiments, the reduction of perimenopausal depression is characterized by a two category change HAM-A severity classification compared to prior to the treatment. In some embodiments, the reduction of perimenopausal depression is characterized by a three category change HAM-A severity classification compared to prior to the treatment.

The General Anxiety Disorder 7 (GAD-7) is an anxiety rating scale consisting of 7 (Spitzer R. L. et al., A brief measure for assessing generalized anxiety disorder. Arch Inern Med. 2006; 166: 1092-1097.). Each symptom is rated from 0 (Not at all Sure) to 4 (Nearly Every Day) scale. Total scores range from 0 to 21 with higher scores indicating greater severity. The total score is used to categorize the severity of anxiety: minimal anxiety (total score between 0-4), mild anxiety (total score between 5-9), moderate anxiety (total score between 10-14) and severe anxiety (total score between 15-21).

In some embodiments, after the treatment the patient experiences a substantial reduction of perimenopausal depression that is characterized by at least about a 20% decline in total GAD-7 value compared to prior to the treatment. In some embodiments, the reduction of perimenopausal depression is characterized by a decline in GAD-7 value ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction of perimenopausal depression that is characterized by at least a one point decline in GAD-7 value compared to prior to the treatment. In some embodiments, the reduction of perimenopausal depression is characterized by a decline in GAD-7 value ranging from about one point to about ten points, for example, about one point, about two points, about three points, about four points, about five points, about six points, about seven points, about eight points, about nine points and about ten points compared to prior to the treatment. In some embodiments, the reduction of perimenopausal depression is characterized by a decline GAD-7 value of about two points. In some embodiments, the reduction of perimenopausal depression is characterized by a decline in GAD-7 value of about three points. In some embodiments, the reduction of perimenopausal depression is characterized by a decline in GAD-7 value of about four points. In some embodiments, the reduction of perimenopausal depression is characterized by a decline in GAD-7 value of about five points. In some embodiments, the reduction of perimenopausal depression is characterized by a decline GAD-7 value of about six points. In some embodiments, the reduction of perimenopausal depression is characterized by a decline in GAD-7 value of about seven points. In some embodiments, the reduction of perimenopausal depression is characterized by a decline in GAD-7 value of about eight points. In some embodiments, the reduction of perimenopausal depression is characterized by a decline in GAD-7 value of about nine points. In some embodiments, the reduction of perimenopausal depression is characterized by a decline in GAD-7 value of about ten points.

In some embodiments, after the treatment the patient experiences a substantial reduction of perimenopausal depression that is characterized by at least a one category change in GAD-7 severity classification compared to prior to the treatment. In some embodiments, the reduction of perimenopausal depression is characterized by a one category change GAD-7 severity classification compared to prior to the treatment. In some embodiments, the reduction of perimenopausal depression is characterized by a two category change GAD-7 severity classification compared to prior to the treatment. In some embodiments, the reduction of perimenopausal depression is characterized by a three category change GAD-7 severity classification compared to prior to the treatment.

The Clinical Global Impression (CGI) (Guy 1976 (Guy W (1976), ECDEU Assessment Manual for Psychopharmacology, Revised. Rockville, Md.: US Department of Health, Education and Welfare) consists of three subscales: the CGI-Severity (CGI-S), the CGI-Improvement (CGI-I) and Efficacy Index. The CGI-S assesses the clinician's impression of the patient's current mental illness. A treating clinician categorizes the severity of the patient's current mental illness on a 7-point scale: 1 (normal, not at all ill), 2 (borderline mentally ill), 3 (mildly ill), 4 (moderately ill), 5 (markedly ill), 6 (severely ill), and 7 (among the most extremely ill patients). The CGI-I assesses the participant's improvement (or worsening) from baseline. A treating clinician categorizes the patient's condition relative to Baseline (e.g., prior to administering an antidepressant) on a 7-point scale: 1 (very much improved), 2 (much improved), 3 (minimally improved), 4 (no change), 5 (minimally worse), 6 (much worse), and 7 (very much worse).

In some embodiments, after the treatment the patient experiences a substantial reduction of perimenopausal depression that is characterized by at least a one point decline in CGI-S value compared to prior to the treatment. In some embodiments, the reduction of perimenopausal depression is characterized by a one point decline in CGI-S value compared to prior to the treatment. In some embodiments, the reduction of perimenopausal depression is characterized by a two point decline in CGI-S value compared to prior to the treatment. In some embodiments, the reduction of perimenopausal depression is characterized by a three point decline in CGI-S value compared to prior to the treatment.

In some embodiments, after the treatment the patient experiences a substantial reduction of perimenopausal depression that is characterized by at least a one point decline in CGI-I value compared to prior to the treatment. In some embodiments, the reduction of perimenopausal depression is characterized by a one point decline in CGI-I value compared to prior to the treatment. In some embodiments, the reduction of perimenopausal depression is characterized by a two point decline in CGI-I value compared to prior to the treatment. In some embodiments, the reduction of perimenopausal depression is characterized by a three point decline in CGI-I value compared to prior to the treatment.

The Symptoms of Depression Questionnaire (SDQ) is a 44-item, self-report scale that consists of five subscales: SDQ-1, SDQ-2, SDQ-3, SDQ-4 and SDQ-5. SDQ-1 includes items related to lassitude, mood, and cognitive functioning. SDQ-2 includes items related to anxiety, agitation, irritability, and anger. SDQ-3 includes items related to suicidal ideation. SDQ-4 assesses disruptions in sleep quality. SDQ-5 includes items on changes in appetite and weight. SDQ is used to assess symptom severity across several subtypes of depression (Pedrelli, P., et al., Reliability and Validity of the Symptoms of Depression Questionnaire (SDQ), CNS Spectr. 2014 Dec.; 19(6), pages 535-546.). The items are rated on a 6-point scale. Each item is rated based on a participant's perception of what is normal for the individual (score=2), what is better than normal (score=1), and what is worse than normal (scores=3-6). Total scores range from 0 to 264 with higher scores indicating greater severity.

In some embodiments, after the treatment the patient experiences a substantial reduction of perimenopausal depression that is characterized by at least about a 10% decline in total SDQ scale value compared to prior to the treatment. In some embodiments, the reduction of perimenopausal depression is characterized by a decline in total SDQ scale value ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction of perimenopausal depression that is characterized by at least about a 10% decline in at least one subscale value selected from SDQ-1, SDQ-2, SDQ-3, SDQ-4 and SDQ-5 compared to prior to the treatment. In some embodiments, the reduction of perimenopausal depression is characterized by a decline in at least one subscale value selected from SDQ-1, SDQ-2, SDQ-3, SDQ-4 and SDQ-5 value ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction of perimenopausal depression that is characterized by at least a one point decline in Global PSQI (described above) score compared to prior to the treatment. In some embodiments, the reduction of perimenopausal depression is characterized by a one point decline in Global PSQI score compared to prior to the treatment. In some embodiments, the reduction of perimenopausal depression is characterized by a two point decline in Global PSQI score compared to prior to the treatment. In some embodiments, the reduction of perimenopausal depression is characterized by a three point decline in Global PSQI score compared to prior to the treatment.

In some embodiments, after the treatment the patient experiences a substantial reduction of perimenopausal symptoms that is characterized by partial remission of the patient's perimenopausal symptoms. In some embodiments, the patient experiences a substantial reduction of perimenopausal depression that is characterized by partial remission of the patient's perimenopausal depression. In some embodiments, partial remission of perimenopausal depression is where the symptoms of the immediately previous depressive episode are present, but full criteria are not met, or there is a period lasting less than 2 months without any significant symptoms of a major depressive episode following the end of such an episode (i.e., the DSM-5's definition of partial remission).

In some embodiments, after the treatment the patient experiences a substantial reduction of perimenopausal depression that is characterized by full remission of the patient's depression. In some embodiments, full remission is where during the past 2 months, no significant signs or symptoms of the disturbance were present (i.e., the DSM-5's definition of full remission).

In some embodiments, the dosing frequency and dose amount per administration of the GABA-A PAM are selected to provide therapeutic effects for the treatment of the sexual symptoms of perimenopause or menopause. In some embodiments, the present invention provides a method of treating the sexual symptoms of perimenopause or menopause such that after the treatment the patient experiences a substantial reduction in the sexual symptoms of perimenopause compared to prior to said treatment. In some embodiments, the sexual symptoms are selected from reduced libido and vaginal dryness.

The revised Sabbatsberg Sexual Self-Rating Scale (SRS) is a questionnaire to evaluate sexual functioning consisting of 12 items (Garratt A M, et al., Measuring sexual functioning in premenopausal women. BJOG 1995; 102:311-316.). The SRS has six domains: sexual interest, sexual activity, satisfaction of sexual life, experience of sexual pleasure, orgasm capacity, and sexual relevancy. Each question has five possible answers, score from 0 to 4 points (from the lowest to the highest sexual satisfaction rating). The range of possible composite scores is 0 to 48, and the composite score is transformed to a scale of 0 to 100, which represents the percentage of the total possible score.

In some embodiments, after the treatment the patient experiences a substantial reduction in the sexual symptoms of perimenopause or menopause that is characterized by at least about a 30% decline in total SRS value compared to prior to the treatment. In some embodiments, the reduction in the sexual symptoms of perimenopause or menopause is characterized by a decline in SRS value ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction in the sexual symptoms of perimenopause or menopause that is characterized by at least a one point decline in SRS value compared to prior to the treatment. In some embodiments, the reduction in the sexual symptoms of perimenopause or menopause is characterized by a decline in SRS value ranging from about one point to about ten points, for example, about one point, about two points, about three points, about four points, about five points, about six points, about seven points, about eight points, about nine points and about ten points compared to prior to the treatment. In some embodiments, the reduction in the sexual symptoms of perimenopause or menopause is characterized by a decline SRS value of about two points. In some embodiments, the reduction in the sexual symptoms of perimenopause or menopause is characterized by a decline in SRS value of about three points. In some embodiments, the reduction in the sexual symptoms of perimenopause or menopause is characterized by a decline in SRS value of about four points. In some embodiments, the reduction in the sexual symptoms of perimenopause or menopause is characterized by a decline in SRS value of about five points.

In some embodiments, the reduction in the sexual symptoms of perimenopause or menopause is characterized by a decline SRS value of about six points. In some embodiments, the reduction in the sexual symptoms of perimenopause or menopause is characterized by a decline in SRS value of about seven points. In some embodiments, the reduction in the sexual symptoms of perimenopause or menopause is characterized by a decline in SRS value of about eight points. In some embodiments, the reduction in the sexual symptoms of perimenopause or menopause is characterized by a decline in SRS value of about nine points. In some embodiments, the reduction in the sexual symptoms of perimenopause or menopause is characterized by a decline in SRS value of about ten points.

In some embodiments, after the treatment the patient experiences a substantial reduction in the sexual symptoms of perimenopause or menopause that is characterized by at least about a 10% decline in at least one SRS subscale value selected from sexual interest, sexual activity, satisfaction of sexual life, experience of sexual pleasure, orgasm capacity, and sexual relevancy compared to prior to the treatment. In some embodiments, the reduction in the sexual symptoms of perimenopause or menopause is characterized by a decline in at least one subscale value selected from sexual interest, sexual activity, satisfaction of sexual life, experience of sexual pleasure, orgasm capacity, and sexual relevancy value ranging from about 10% to about 100%, for example, about 20%, about 30%, about 40%, about 50%, about 60%, about 70%, about 80%, about 90%, and about 100%, compared to prior to the treatment.

In some embodiments, the dosing frequency and dose amount per administration of the GABA-A PAM are selected to provide therapeutic effects for the treatment of the vasomotor symptoms of perimenopause or menopause. In some embodiments, the present invention provides a method of treating the vasomotor symptoms of perimenopause or menopause such that after the treatment the patient experiences a substantial reduction in the vasomotor symptoms of perimenopause or menopause compared to prior to said treatment. The vasomotor symptoms (such as hot flushes) of perimenopause or menopause may be measured using any suitable method that is known to those skilled in the art. In some embodiments, the vasomotor symptoms of perimenopause or menopause are measured using a patient diary, wherein in the patient tracks the total daily number and intensity (mild, moderate or severe) of daytime hot flushes and nighttime hot flushes (a.k.a., night sweats). In some embodiments, the vasomotor symptoms of perimenopause or menopause are measured using the average intensity of the patient's hot flushes according to the following formula:

$$Average intensity of hot flushes = \frac{Sum of hot flush intensity}{Total number of hot flushes}$$

where mild, moderate and severe intensity hot flush are givens value of 1, 2 and 3, respectively.

In some embodiments, after the treatment the patient experiences a substantial reduction of the vasomotor symptoms of perimenopause or menopause that is characterized by a reduction in the patient's daily number of hot flushes compared to prior to the treatment. In some embodiments, after the treatment the patient experiences a substantial reduction of the vasomotor symptoms of perimenopause or menopause that is characterized by at least about a 10% decline in the patient's daily number of hot flushes compared to prior to the treatment. In some embodiments, the reduction of the vasomotor symptoms of perimenopause or menopause is characterized by a decline in the patient's daily number of hot flushes ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction of the vasomotor symptoms of perimenopause or menopause that is characterized by a reduction in the average intensity of the patient's hot flushes compared to prior to the treatment. In some embodiments, after the treatment the patient experiences a substantial reduction of the vasomotor symptoms of perimenopause or menopause that is characterized by at least about a 10% decline in the average intensity of the patient's hot flushes compared to prior to the treatment. In some embodiments, the reduction of the vasomotor symptoms of perimenopause or menopause is characterized by a decline in the average intensity of the patient's hot flushes ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction of the vasomotor symptoms of perimenopause or menopause that is characterized by a reduction in the patient's daily number of daytime hot flushes compared to prior to the treatment. In some embodiments, after the treatment the patient experiences a substantial reduction of the vasomotor symptoms of perimenopause or menopause that is characterized by at least about a 10% decline in the patient's daily number of daytime hot flushes compared to prior to the treatment. In some embodiments, the reduction of the vasomotor symptoms of perimenopause or menopause is characterized by a decline in the patient's daily number of daytime hot flushes ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction of the vasomotor symptoms of perimenopause or menopause that is characterized by a reduction in the average intensity of the patient's daytime hot flushes compared to prior to the treatment. In some embodiments, after the treatment the patient experiences a substantial reduction of the vasomotor symptoms of perimenopause or menopause that is characterized by at least about a 10% decline in the average intensity of the patient's daytime hot flushes compared to prior to the treatment. In some embodiments, the reduction of the vasomotor symptoms of perimenopause or menopause is characterized by a decline in the average intensity of the patient's daytime hot flushes ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction of the vasomotor symptoms of perimenopause or menopause that is characterized by a reduction in the patient's daily number of nighttime hot flushes compared to prior to the treatment. In some embodiments, after the treatment the patient experiences a substantial reduction of the vasomotor symptoms of perimenopause or menopause that is characterized by at least about a 10% decline in the patient's daily number of nighttime hot flushes compared to prior to the treatment. In some embodiments, the reduction of the vasomotor symptoms of perimenopause or menopause is characterized by a decline in the patient's daily number of nighttime hot flushes ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction of the vasomotor symptoms of perimenopause or menopause that is characterized by a reduction in the average intensity of the patient's nighttime hot flushes compared to prior to the treatment. In some embodiments, after the treatment the patient experiences a substantial reduction of the vasomotor symptoms of perimenopause or menopause that is characterized by at least about a 10% decline in the average intensity of the patient's nighttime hot flushes compared to prior to the treatment. In some embodiments, the reduction of the vasomotor symptoms of perimenopause or menopause is characterized by a decline in the average intensity of the patient's nighttime hot flushes ranging from about 10% to about 100%, for example, about 20%, about 30%, about 40%, about 50%, about 60%, about 70%, about 80%, about 90%, and about 100%, compared to prior to the treatment.

In some embodiments, the dosing frequency and dose amount per administration of the GABA-A PAM are selected to provide therapeutic effects for the treatment of the sleep symptoms of perimenopause or menopause. In some embodiments, the sleep symptoms are selected from sleep disturbance and insomnia. In some embodiments, the dosing frequency and dose amount per administration of the GABA-A PAM are selected to provide therapeutic effects for the treatment of insomnia characterized by difficulties with sleep initiation. In some embodiments, the dosing frequency and dose amount per administration of the GABA-A PAM are selected to provide therapeutic effects for the treatment of insomnia in a perimenopausal depression patient with insomnia.

In some embodiments, after treatment for at least one week the patient experiences a substantial reduction of insomnia compared to prior to the treatment. According to this embodiment, the substantial reduction in insomnia may be expressed using any of the methods described herein (for example, an improvement in polysomnography parameters, such as a decline in latency to persistent sleep (LPS) compared to prior to the treatment, decline in wake time after sleep onset (WASO) compared to prior to the treatment, etc.).

The sleep parameters described herein (including wake time after sleep onset, total sleep time, sleep efficiency and latency to persistent sleep) may be measured by polysomnography using methods that are known to those skilled in the art.

Wake time after sleep onset is the wakefulness time occurring after defined sleep onset. In some embodiments, after the treatment the patient experiences a substantial reduction of insomnia that is characterized by at least about a 30% decline in wake time after sleep onset (WASO) compared to prior to the treatment. In some embodiments, the reduction of insomnia is characterized by a decline in WASO ranging from about 30% to about 100%, for example, about 30%, about 40%, about 50%, about 60%, about 70%, about 80%, about 90%, and about 100%, compared to prior to the treatment.

Total Sleep Time is the amount of actual sleep time in a sleep episode; i.e., the total sleep episode less the awake time. In some embodiments, after the treatment the patient experiences a substantial reduction of insomnia that is characterized by at least about a 30% increase in Total Sleep Time (TST) compared to prior to the treatment. In some embodiments, the reduction of insomnia is characterized by an increase in TST ranging from about 30% to about 100%, for example, about 30%, about 40%, about 50%, about 60%, about 70%, about 80%, about 90%, and about 100%, compared to prior to the treatment.

Sleep efficiency is the percentage of total time in bed actually spent in sleep. An increase in sleep efficiency correlates to an improvement in insomnia. In some embodiments, after the treatment the patient experiences a substantial reduction of insomnia that is characterized by at least about a 30% increase in sleep efficiency (SE) compared to prior to the treatment. In some embodiments, the reduction of insomnia is characterized by an increase in SE value ranging from about 30% to about 100%, for example, about 30%, about 40%, about 50%, about 60%, about 70%, about 80%, about 90%, and about 100%, compared to prior to the treatment.

Latency to persistent sleep is the length of time that it takes to accomplish the transition from full wakefulness to sleep. In some embodiments, after the treatment the patient experiences a substantial reduction of insomnia that is characterized by at least about a 30% decrease in latency to persistent sleep (LPS) compared to prior to the treatment. In some embodiments, the reduction of insomnia is characterized by a decline in LPS value ranging from about 30% to about 100%, for example, about 30%, about 40%, about 50%, about 60%, about 70%, about 80%, about 90%, and about 100%, compared to prior to the treatment.

The Pittsburgh Sleep Quality Index (PSQI) is a 19-item self-report scale that assesses sleep quality and disturbances for the month preceding the assessment (Buysse D. J., The Pittsburgh Sleep Quality Index: a New Instrument for Psychiatric Practice and Research. Psychiatry Res. 1989 May; 28(2), pages 193-213.). The scale generates seven “component” scores that differentiate “poor” from “good” sleep quality: subjective sleep quality, sleep latency, sleep duration, habitual sleep efficiency, sleep disturbances, use of sleeping medication, and daytime dysfunction. The sum of scores for these seven components yields the Global PSQI score. A Global PSQI score of “5” or greater indicates poor sleep quality. In some embodiments, after the treatment the patient experiences a substantial reduction of insomnia that is characterized by at least a one point decline in Global Pittsburgh Sleep Quality Index (PSQI) score compared to prior to the treatment. In some embodiments, the reduction of insomnia is characterized by a one point decline in Global PSQI score compared to prior to the treatment. In some embodiments, the reduction of insomnia is characterized by a two point decline in Global PSQI score compared to prior to the treatment. In some embodiments, the reduction of insomnia is characterized by a three point decline in Global PSQI score compared to prior to the treatment.

The Epworth Sleepiness Scale (ESS) is also useful for determining the treatment of insomnia. In scoring the ESS, each item is rated on a 4-point scale from 0=would never doze to 3=high chance of dozing. The item scores are summed to produce a total score (range 0-24). A sum of 10 or more from the 8 individual scores reflects above normal daytime sleepiness and need for further evaluation. In some embodiments, after the treatment the patient experiences a substantial reduction of insomnia that is characterized by at least a one point increase in ESS value compared to prior to the treatment. In some embodiments, the reduction of insomnia is characterized by a one point increase in ESS value compared to prior to the treatment. In some embodiments, the reduction of insomnia is characterized by a two point increase in ESS value compared to prior to the treatment. In some embodiments, the reduction of insomnia is characterized by a three point increase in ESS value compared to prior to the treatment.

The Insomnia Severity Index (ISI) may be used to determine the treatment of insomnia. For example, the Insomnia Severity Index has seven questions, where answers provide a total score ranging from 0 to 28. A total score of 0 to 7 indicates no significant insomnia; 8 to 14 indicates subthreshold insomnia; 15 to 21 indicates clinical insomnia—moderate severity; and 22-28 indicates clinical insomnia—severe. In some embodiments, after the treatment the patient experiences a substantial reduction of insomnia that is characterized by at least a one point decrease in Insomnia Severity Index scale value compared to prior to the treatment. In some embodiments, the reduction of insomnia is characterized by a one point decrease in ISI value compared to prior to the treatment. In some embodiments, the reduction of insomnia is characterized by a two point decrease in ISI value compared to prior to the treatment. In some embodiments, the reduction of insomnia is characterized by a three point decrease in ISI value compared to prior to the treatment.

The Leeds Sleep Evaluation Questionnaire (LSEQ) may be used to determine the treatment of insomnia. The LSEQ is a 10-item, subjective, self-report measure designed to assess changes in sleep quality over the course of a drug treatment intervention. The LSEQ has four domains: Ease of Initiating Sleep (three items), Quality of Sleep (two items), Ease of Waking (two items) and Behavior Following Wakefulness (three items). LSEQ uses a visual analogue scale where the respondents place markers on a group of 10-cm lines representing the changes have experience in a variety of symptoms since beginning treatment. Lines extend between extremes such as “more difficult than usual” and “easier than usual” (item 6 related to ease of waking). Responses are measured using a 100-mm scale and are then averaged to provide a score for each domain. The average scores can be used to evaluate the efficacy and sleep-related side effects of drug treatment. In some embodiments, after the treatment the patient experiences a substantial reduction of insomnia that is characterized by at least about a 10% improvement in total LSEQ value compared to prior to the treatment. In some embodiments, the reduction of insomnia is characterized by an increase in total LSEQ value ranging from about 10% to about 100%, for example, about 10% about, 20%, about 30%, about 40%, about 50%, about 60%, about 70%, about 80%, about 90%, and about 100%, compared to prior to the treatment. In some embodiments, after the treatment the patient experiences a substantial reduction of insomnia that is characterized by at least about a 10% improvement in Ease of Initiating Sleep LSEQ domain value compared to prior to the treatment. In some embodiments, the reduction of insomnia is characterized by an increase in Ease of Initiating Sleep LSEQ domain value ranging from about 10% to about 100%, for example, about 10% about, 20%, about 30%, about 40%, about 50%, about 60%, about 70%, about 80%, about 90%, and about 100%, compared to prior to the treatment. In some embodiments, after the treatment the patient experiences a substantial reduction of insomnia that is characterized by at least about a 10% improvement in Quality of Sleep LSEQ domain value compared to prior to the treatment. In some embodiments, the reduction of insomnia is characterized by an increase in Quality of Sleep LSEQ domain value ranging from about 10% to about 100%, for example, about 10% about, 20%, about 30%, about 40%, about 50%, about 60%, about 70%, about 80%, about 90%, and about 100%, compared to prior to the treatment. In some embodiments, after the treatment the patient experiences a substantial reduction of insomnia that is characterized by at least about a 10% improvement in Ease of Waking LSEQ domain value compared to prior to the treatment. In some embodiments, the reduction of insomnia is characterized by an increase in Ease of Waking LSEQ domain value ranging from about 10% to about 100%, for example, about 10% about, 20%, about 30%, about 40%, about 50%, about 60%, about 70%, about 80%, about 90%, and about 100%, compared to prior to the treatment. In some embodiments, after the treatment the patient experiences a substantial reduction of insomnia that is characterized by at least about a 10% improvement in Behavior Following Wakefulness LSEQ domain value compared to prior to the treatment. In some embodiments, the reduction of insomnia is characterized by an increase in Behavior Following Wakefulness LSEQ domain value ranging from about 10% to about 100%, for example, about 10% about, 20%, about 30%, about 40%, about 50%, about 60%, about 70%, about 80%, about 90%, and about 100%, compared to prior to the treatment.

The Athens Insomnia Scale (AIS) may be used to determine the treatment of insomnia. The AIS scale assess the severity of insomnia using the diagnostic criteria set forth by the International Classification of Diseases (ICD-10). The eight-item questionnaire evaluates sleep onset, night and early-morning waking, sleep time, sleep quality, frequency and duration of complaints, distress cause by the experience and interference with daily functions. Respondents use Likert-type scales to show how severely certain sleep difficulties have affected them in the past month. Scores range from 0 (meaning that the item in question has not been a problem to 3 (indicating more acute sleep difficulties) where answers provide a total score ranging from 0 to 24. In some embodiments, after the treatment the patient experiences a substantial reduction of insomnia that is characterized by at least a one point decrease in total AIS value compared to prior to the treatment. In some embodiments, the reduction of insomnia is characterized by a one point decrease in total AIS value compared to prior to the treatment. In some embodiments, the reduction of insomnia is characterized by a two point decrease in total AIS value compared to prior to the treatment. In some embodiments, the reduction of insomnia is characterized by a three point decrease in total AIS value compared to prior to the treatment. In some embodiments, the reduction of insomnia is characterized by a four point decrease in total AIS value compared to prior to the treatment. In some embodiments, the reduction of insomnia is characterized by a five point decrease in total AIS value compared to prior to the treatment. In some embodiments, the reduction of insomnia is characterized by a six point decrease in total AIS value compared to prior to the treatment. In some embodiments, the reduction of insomnia is characterized by a seven point decrease in total AIS value compared to prior to the treatment. In some embodiments, the reduction of insomnia is characterized by an eight point decrease in total AIS value compared to prior to the treatment. In some embodiments, the reduction of insomnia is characterized by a nine point decrease in total AIS value compared to prior to the treatment. In some embodiments, the reduction of insomnia is characterized by a ten point decrease in total AIS value compared to prior to the treatment.

The Sleep Quality Index (SQI) may be used to determine the treatment of insomnia. The SQI is an eight item questionnaire with three categories weighted 0, 1, or 2 for each item (Urponen H., et al. (1991) Sleep Quality and Health: Description of the Sleep Quality Index. In: Peter J. H., Penzel T., Podszus T., von Wichert P. (eds) Sleep and Health Risk. Springer, Berlin. Heidelberg). The value of SQI varies from 0 to 16 with higher scores indicating more severe sleep disturbance. In some embodiments, after the treatment the patient experiences a substantial reduction of insomnia that is characterized by at least a one point decrease in total SQI value compared to prior to the treatment. In some embodiments, the reduction of insomnia is characterized by a one point decrease in total SQI value compared to prior to the treatment. In some embodiments, the reduction of insomnia is characterized by a two point decrease in total SQI value compared to prior to the treatment. In some embodiments, the reduction of insomnia is characterized by a three point decrease in total SQI value compared to prior to the treatment. In some embodiments, the reduction of insomnia is characterized by a four point decrease in total SQI value compared to prior to the treatment. In some embodiments, the reduction of insomnia is characterized by a five point decrease in total SQI value compared to prior to the treatment. In some embodiments, the reduction of insomnia is characterized by a six point decrease in total SQI value compared to prior to the treatment. In some embodiments, the reduction of insomnia is characterized by a seven point decrease in total SQI value compared to prior to the treatment. In some embodiments, the reduction of insomnia is characterized by an eight point decrease in total SQI value compared to prior to the treatment. In some embodiments, the reduction of insomnia is characterized by a nine point decrease in total SQI value compared to prior to the treatment. In some embodiments, the reduction of insomnia is characterized by a ten point decrease in total SQI value compared to prior to the treatment.

In some embodiments, the dosing frequency and dose amount per administration of the GABA-A PAM are selected to provide therapeutic effects for the treatment of the cognitive symptoms of perimenopause or menopause. In some embodiments, the present invention provides a method of treating the cognitive symptoms of perimenopause or menopause such that after the treatment the patient experiences a substantial reduction in the mood symptoms of perimenopause or menopause. In some embodiments, the cognitive symptoms are selected from memory loss and difficulty concentrating.

The Massachusetts General Hospital Cognitive and Physical Functioning Questionnaire (CPFQ) may be used to determine the treatment of the cognitive symptoms of perimenopause or menopause. The CPFQ is seven item questionnaire each item rated on a scale from 1 to 6, with 1 indicating greater than normal functioning, 2 indicating normal functioning and higher numbers indicating poorer function (Fava, M, et al., Reliability and Validity of the Massachusetts General Hospital Cognitive and Physical Functioning Questionnaire, Psychothera Psychosom. 2009, 78, 91-97). The value of total CPFQ varies from 7 to 42 with higher scores indicating poorer functioning. In some embodiments, after the treatment the patient experiences a substantial reduction of the cognitive symptoms of perimenopause or menopause that is characterized by at least a one point decrease in total CPFQ value compared to prior to the treatment. In some embodiments, the reduction of the cognitive symptoms of perimenopause or menopause is characterized by a one point decrease in total CPFQ value compared to prior to the treatment. In some embodiments, the reduction of the cognitive symptoms of perimenopause or menopause is characterized by a two point decrease in total CPFQ value compared to prior to the treatment. In some embodiments, the reduction of the cognitive symptoms of perimenopause or menopause is characterized by a three point decrease in total CPFQ value compared to prior to the treatment. In some embodiments, the reduction of the cognitive symptoms of perimenopause or menopause is characterized by a four point decrease in total CPFQ value compared to prior to the treatment. In some embodiments, the reduction of the cognitive symptoms of perimenopause or menopause is characterized by a five point decrease in total CPFQ value compared to prior to the treatment. In some embodiments, the reduction of the cognitive symptoms of perimenopause or menopause is characterized by a six point decrease in total CPFQ value compared to prior to the treatment. In some embodiments, the reduction of the cognitive symptoms of perimenopause or menopause is characterized by a seven point decrease in total CPFQ value compared to prior to the treatment. In some embodiments, the reduction of the cognitive symptoms of perimenopause or menopause is characterized by an eight point decrease in total CPFQ value compared to prior to the treatment. In some embodiments, the reduction of the cognitive symptoms of perimenopause or menopause is characterized by a nine point decrease in total CPFQ value compared to prior to the treatment. In some embodiments, the reduction of the cognitive symptoms of perimenopause or menopause is characterized by a ten point decrease in total CPFQ value compared to prior to the treatment.

In some embodiments, the method of treating the symptoms of perimenopause or menopause further includes a step of titrating the dose of Compound 1 until a maintenance dose is achieved in the patient. In some embodiments, the titration is conducted for at least about one week until a maintenance dose is achieved in the patient. In one embodiment, the titration is conducted for about 2 weeks until a maintenance dose is achieved in the patient. In some embodiments, the titration is conducted for about 7 days to about 30 days until a maintenance dose is achieved in the patient. In some embodiments, the titration is conducted for about 12 days to about 20 days until a maintenance dose is achieved in the patient. In some embodiments, during the titration step, a constant daily dose of Compound 1 is provided. In further embodiments, the constant daily dose of Compound 1 is provided for at least two weeks.

The daily dose can be titrated in one or more steps. The daily dosage can be titrated by increasing a single daily dosage, or each dose of a twice-daily dosing regimen. The amount a dosage is stepped, where there are multiple titration steps, can be the same, or can be different.

In some embodiments, the titration is initiated with from about 15 mg to about 100 mg of Compound 1 or a pharmaceutically acceptable salt thereof, including about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg and about 100 mg including all ranges therebetween once or twice daily. In some embodiments, the titration is initiated with about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg and about 100 mg of Compound 1 or a pharmaceutically acceptable salt thereof once or twice daily. In some embodiments, doses can be adjusted in 5-30 mg increments every 1 to 4 days. In some embodiments, doses can be adjusted in 5-30 mg increments every week. In some embodiments, the titration is conducted for at least about one week, 2 weeks, 3 weeks, 4 weeks, 5 weeks, 6 weeks, about 7 weeks, about 8 weeks, about 9 weeks or about 10 weeks prior to the maintenance dose

In some embodiments, ascending doses of Compound 1 are administered during the titration until a maintenance dose is achieved in the patient. In some embodiments, ascending doses of the Compound 1 are administered during the titration until an effective amount of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg or about 120 mg is achieved in the patient.

In some embodiments, patients are initially administered about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg or about 100 mg of Compound 1 or a pharmaceutically acceptable salt once or twice a day and are titrated to a maintenance dose of about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg or about 120 mg once or twice a day. In some embodiments, patients are initially administered from about 15 mg to about 100 mg of Compound 1 or a pharmaceutically acceptable salt, including about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg and about 100 mg, including all ranges therebetween once or twice a day and are titrated to a maintenance dose of from about 20 mg to about 120 mg, including about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg or about 120 mg including all ranges therebetween, once or twice a day.

In some embodiments, the present disclosure provides a method of treating the symptoms of perimenopause or menopause that includes the steps of: (a) administering an initial daily dose of Compound 1 for at least one week and (b) administering a maintenance daily dose for at least one week. In some embodiments, the initial daily dose is greater than the maintenance daily dose. In some embodiments, the initial daily dose is less than the maintenance daily dose. In some embodiments, the initial daily dose is administered for two weeks and the maintenance daily dose is administered is administered for at least one month.

In some embodiments, the present disclosure provides a method of treating the symptoms of perimenopause or menopause that includes the steps of:
(a) administering a loading dose of Compound 1 and
(b) administering a maintenance dose of Compound 1.

In some embodiments, the loading dose is administered for about 1 day to about 14 days, including about 1 day, about 2 days, about 3 days, about 4 days, about 5 days, about 6 days, about 7 days, about 8 days, about 9 days, about 10 days, about 11 days, about 12 days, about 13 days and about 14 days, including all ranges therebetween. In some embodiments, the loading dose is administered for about 1 day, about 2 days, about 3 days, about 4 days, about 5 days, about 6 days, about 7 days, about 8 days, about 9 days, about 10 days, about 11 days, about 12 days, about 13 days or about 14 days.

In some embodiments, the methods comprise a loading dose of from about 30 mg to about 120 mg, including about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, and about 120 mg including all ranges therebetween. In some embodiments, the methods comprise a loading dose of about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg,

In some embodiments, the maintenance dose is administered for from about 1 month to about 36 months, including about 1 month, about 2 months, about 3 months, about 4 months, about 5 months, about 6 months, about 7 months, about 8 months, about 9 months, about 10 months, about 11 months, about 12 months, about 18 months, about 24 months, about 30 months, or about 36 months including all ranges therebetween. In some embodiments, the maintenance dose is administered for about 1 month, about 2 months, about 3 months, about 4 months, about 5 months, about 6 months, about 7 months, about 8 months, about 9 months, about 10 months, about 11 months, about 12 months, about 18 months, about 24 months, about 30 months, or about 36 months.

In some embodiments, the methods comprise a maintenance dose of from about 30 mg to about 120 mg, including about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, and about 120 mg including all ranges therebetween, once or twice a day. In some embodiments, the methods comprise a maintenance dose of from about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg, once or twice a day.

In some embodiments, the loading dose administration methods further comprise a cessation period after administration of the loading dose and prior to administration of the maintenance dose.

In some embodiments, the cessation period is about one day, about two days, about three days, about four days, about five days, about six days, or about seven days. In some embodiments, the cessation period is from about one day to about seven days, including about one day, about two days, about three days, about four days, about five days, about six days, and about seven days, including all ranges therebetween.

In some embodiments, the cessation period is about one week, about two weeks, about three weeks, about four weeks, about five weeks, about six weeks, about seven weeks, or about eight weeks. In some embodiments, the cessation period is from about one week to about eight weeks, including about one week, about two weeks, about three weeks, about four weeks, about five weeks, about six weeks, about seven weeks, and about eight weeks including all ranges therebetween.

In some embodiments, the methods of the present disclosure comprise continuous administration of Compound 1 or a pharmaceutically acceptable salt thereof for a specified interval (for example, one week) followed by a cessation period wherein the patient is not administered Compound 1. In some embodiments, the methods of the present disclosure comprise continuous administration of Compound 1 or a pharmaceutically acceptable salt thereof for from about one week to about eight weeks, including about one week, about two weeks, about three weeks, about four weeks, about five weeks, about six weeks, about seven weeks, and about eight weeks including all ranges therebetween. In some embodiments, the methods of the present disclosure comprise continuous administration of Compound 1 or a pharmaceutically acceptable salt thereof for about one week, about two weeks, about three weeks, about four weeks, about five weeks, about six weeks, about seven weeks, or about eight weeks.

In some embodiments, the methods of the present disclosure comprise continuous administration of Compound 1 or a pharmaceutically acceptable salt thereof for from about one month to about 36 months, including about one month, about two months, about three months, about four months, about five months, about six months, about seven months, about eight months, about 9 months, about 10 months, about 11 months, about 12 months, about 18 months, about 24 months, about 30 months, and about 36 months including all ranges therebetween. In some embodiments, the methods of the present disclosure comprise continuous administration of Compound 1 or a pharmaceutically acceptable salt thereof for about one month, about two months, about three months, about four months, about five months, about six months, about seven months, about eight months, about nine months, about ten months, about eleven months, about twelve months, about 18 months, about 24 months, about 30 months, or about 36 months.

In some embodiments, the methods of the present disclosure comprise intermittent administration of Compound 1 or a pharmaceutically acceptable salt thereof. As used herein, intermittent administration means cycling a patient in need thereof on and off treatment with Compound 1 or a pharmaceutically acceptable salt thereof for specified time intervals.

In some embodiments, intermittent administration comprises:
(a) administering Compound 1 for a first administration period;
(b) after the first administration period (a), not administering Compound 1 for a cessation period;
(c) after the cessation period (b), administering Compound 1 for a second administration period.

In some embodiments, the intermittent administration further comprises one or more additional cessation periods (for example, a second cessation period) and/or administration periods (for example, a third administration period). In such embodiments, the present disclosure contemplates embodiments wherein the additional cessation and/or administration periods have the durations described herein for the first administration period and the cessation period.

In some embodiments, two or more of the periods (a), (b) and (c) are the same (for example, the first administration period, cessation period and second administration period are each one week). In some embodiments, the periods (a) and (b) (for example, one week) are the same and the period (c) is different (for example, two weeks). In some embodiments, the periods (a) and (c) are the same and the period (b) is different. In some embodiments, the periods (b) and (c) are the same and the period (a) is different. In some embodiments, the periods (a), (b) and (c) are the different (for example, the first administration period is one week, the cessation period is two weeks and the second administration period is three weeks).

In some embodiments, the first administration period is about one week, about two weeks, about three weeks, about four weeks, about five weeks, about six weeks, about seven weeks, or about eight weeks. In some embodiments, the first administration period is from about one week to about eight weeks, including about one week, about two weeks, about three weeks, about four weeks, about five weeks, about six weeks, about seven weeks, and about eight weeks including all ranges therebetween.

In some embodiments, the cessation period is about one week, about two weeks, about three weeks, about four weeks, about five weeks, about six weeks, about seven weeks, or about eight weeks. In some embodiments, the cessation period is from about one week to about eight weeks, including about one week, about two weeks, about three weeks, about four weeks, about five weeks, about six weeks, about seven weeks, and about eight weeks including all ranges therebetween.

In some embodiments, the second administration period is about one week, about two weeks, about three weeks, about four weeks, about five weeks, about six weeks, about seven weeks, or about eight weeks. In some embodiments, the second administration period is from about one week to about eight weeks, including about one week, about two weeks, about three weeks, about four weeks, about five weeks, about six weeks, about seven weeks, or about eight weeks including all ranges therebetween.

In some embodiments, the first administration period, cessation period and second administration period are one week. In some embodiments, the first administration period, cessation period and second administration period are two weeks. In some embodiments, the first administration period, cessation period and second administration period are three weeks. In some embodiments, the first administration period, cessation period and second administration period are four weeks. In some embodiments, the first administration period, cessation period and second administration period are five weeks. In some embodiments, the first administration period, cessation period and second administration period are six weeks. In some embodiments, the first administration period, cessation period and second administration period are seven weeks. In some embodiments, the first administration period, cessation period and second administration period are eight weeks.

In some embodiments, the first administration period is about one week; the cessation period is about three weeks; and the second administration period is about one week.

In some embodiments, the first administration period is about two weeks; the first cessation period is about two weeks; the second administration period is about one week; the second cessation period is about one week and the third administration period is about one week.

In some embodiments, the intermittent administration period (including the administration and cessation periods) is about one month, about two months, about three months, about four months, about five months, about six months, about seven months, about eight months, about nine months, about ten months, about eleven months, about twelve months, about 18 months, about 24 months, about 30 months or about 36 months. In some embodiments, the intermittent administration period is from about one month to about twelve months, including about two months, about three months, about four months, about five months, about six months, about seven months, about eight months, about nine months, about ten months, about eleven months, about twelve months, about 18 months, about 24 months, about 30 months and about 36 months, including all ranges therebetween.

In some embodiments of the present disclosure, Compound 1 or a pharmaceutically acceptable salt thereof is administered to a patient with food. In some embodiments, Compound 1 or a pharmaceutically acceptable salt thereof is administered to a patient about 5 minutes, about 10 minutes, about 15 minutes, about 30 minutes, about 45 minutes, about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours or about 8 hours after food is ingested.

In some embodiments, the food ingested is a high fat and high calorie food. In some embodiments, the caloric content of the high fat and high calorie food is at least about 700 kilocalories (kcal), and at least about 40 percent of the caloric content of the food is from fat. For example, the fat can contribute to about 50 percent of the caloric content of the food of high fat and high calorie. In some embodiments, the caloric content of the high fat and high calorie food is about 900 kilocalories.

In some embodiments, the food ingested is a medium fat and medium calorie food. In some embodiments, the caloric content of the medium fat and medium calorie food is about 300 kcal to about 700 kcal, and between about 20 percent to about 40 percent of the caloric content of the food is from fat. In some embodiments, the caloric content of the medium fat and medium calorie food is about 400 kcal.

In some embodiments, the food ingested is a low fat and low calorie food. In some embodiments, the caloric content of the low fat and low calorie food is between about 100 kcal to about 300 kcal, and the fat content is approximately 3 grams or less, or about 20 percent or less of the caloric content of the food are from fat. In some embodiments, the caloric content of the food of low fat and low calorie is about 100 kilocalories.

In some embodiments of the present disclosure, Compound 1 or a pharmaceutically acceptable salt thereof is administered to a patient after a fasting period. In some embodiments, Compound 1 or a pharmaceutically acceptable salt thereof is administered to a patient after a fasting period of about 1 hour, about 2 hours, about 3 hours, about 4 hours, about 5 hours, about 6 hours, about 7 hours, about 8 hours, about 9 hours, about 10 hours, about 11 hours, or about 12 hours.

In some embodiments of the present disclosure, Compound 1 or a pharmaceutically acceptable salt thereof is administered to a patient without regard to meals. In some embodiments, Compound 1 or a pharmaceutically acceptable salt thereof is administered to a patient at bedtime.

In some embodiments, the methods of the present disclosure comprise controlling the gastrointestinal pH of the patient prior to, concurrently with or after administration of Compound 1. In some embodiments, the gastrointestinal pH of the patient is controlled prior to administration of Compound 1. In some embodiments, the gastrointestinal pH of the patient is controlled after administration of Compound 1.

In some embodiments, the pH is controlled by administering a drug, food or liquid to a patient that decreases gastrointestinal pH prior to, concurrently with or after administration of Compound 1. In some embodiments, the liquid is an acidic beverage (such as a carbonated beverage).

In some embodiments, the pH is controlled by the patient avoiding a drug, food or beverage that increases gastrointestinal pH prior to, concurrently with or after administration of Compound 1. In some embodiments, the drug that increases gastrointestinal pH is a proton pump inhibitor or an orally-administered antacid.

In some embodiments, the initial daily dosing frequency and dose amount per administration of the GABA-A PAM are selected to provide therapeutic effects for the acute treatment of depression and the maintenance daily dosing frequency and dose amount per administration of Compound 1 are selected to provide therapeutic effects for the chronic treatment of depression.

In some embodiments, the initial daily dosing frequency and dose amount per administration of the GABA-A PAM are selected to provide therapeutic effects for the acute treatment of the symptoms of perimenopause or menopause and the maintenance daily dosing frequency and dose amount per administration of the GABA-A PAM are selected to maintain remission of the symptoms of perimenopause or menopause.

In some embodiments, the initial daily dosing frequency and dose amount per administration of the GABA-A PAM are selected to provide therapeutic effects for the acute treatment of the symptoms of perimenopause or menopause and the maintenance the daily dosing frequency and dose amount per administration of the GABA-A PAM are selected to prevent recurrence of the symptoms of perimenopause or menopause.

In some embodiments, the initial daily dose is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg or about 120 mg and the maintenance daily dose is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, or about 115 mg provided the initial daily dose is greater than the maintenance daily dose.

According to some embodiments of the present invention, the methods of the present invention provide therapeutically effective blood plasma levels of Compound 1 for treating the symptoms of perimenopause or menopause. Blood plasma levels of Compound 1 may be expressed using pharmacokinetic parameters that are known to those skilled in the art, such as steady state plasma levels, AUC, Cmax and Cmin.

In some embodiments, the present methods provide steady state plasma levels of Compound 1 that correlate to one or more statistically significant therapeutic effects. In some embodiments, the therapeutically effective steady state plasma levels of Compound 1 provided by the methods of the present invention range from about 1 ng/mL to about 500 ng/mL, including about 1 ng/ml, about 5 ng/mL, about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL about 80 ng/mL, about 85 ng/mL, about 90 ng/mL, about 95 ng/mL, about 100 ng/mL, about 105 ng/mL, about 110 ng/mL, about 115 ng/mL, about 120 ng/mL, about 125 ng/mL, about 130 ng/mL, about 135 ng/mL, about 140 ng/mL, about 145 ng/mL, about 150 ng/mL, about 155 ng/mL, about 160 ng/mL, about 165 ng/mL, about 170 ng/mL, about 175 ng/mL about 180 ng/mL, about 185 ng/mL, about 190 ng/mL, about 195 ng/mL, 200 ng/ml, about 205 ng/mL, about 210 ng/mL, about 215 ng/mL, about 220 ng/mL, about 225 ng/mL, about 230 ng/mL, about 235 ng/mL, about 240 ng/mL, about 245 ng/mL, about 250 ng/mL, about 255 ng/mL, about 260 ng/mL, about 265 ng/mL, about 270 ng/mL, about 275 ng/mL about 280 ng/mL, about 285 ng/mL, about 290 ng/mL, about 295 ng/mL, about 300 ng/mL, about 305 ng/mL, about 310 ng/mL, about 315 ng/mL, about 320 ng/mL, about 325 ng/mL, about 330 ng/mL, about 335 ng/mL, about 340 ng/mL, about 345 ng/mL, about 350 ng/mL, about 355 ng/mL, about 360 ng/mL, about 365 ng/mL, about 370 ng/mL, about 375 ng/mL about 380 ng/mL, about 385 ng/mL, about 390 ng/mL, about 395 ng/mL, about 400 ng/mL, about 405 ng/mL, about 410 ng/mL, about 415 ng/mL, about 420 ng/mL, about 425 ng/mL, about 430 ng/mL, about 435 ng/mL, about 440 ng/mL, about 445 ng/mL, about 450 ng/mL, about 455 ng/mL, about 460 ng/mL, about 465 ng/mL, about 470 ng/mL, about 475 ng/mL about 480 ng/mL, about 485 ng/mL, about 490 ng/mL, about 495 ng/mL, and about 500 ng/mL including all ranges therebetween. In some embodiments, the therapeutically effective steady state plasma levels of Compound 1 provided by the methods of the present invention range from about 50 ng/ml to 500 ng/ml.

In some embodiments, the therapeutically effective steady state plasma levels of Compound 1 is provided by administering a daily dose of Compound 1 or a pharmaceutically acceptable salt thereof of about 15 mg. In further embodiments, the therapeutically effective steady state plasma levels of Compound 1 is provided by administering about 15 mg of Compound 1 or a pharmaceutically acceptable salt thereof once a day.

In some embodiments, the therapeutically effective steady state plasma levels of Compound 1 is provided by administering a daily dose of Compound 1 or a pharmaceutically acceptable salt thereof of about 20 mg. In further embodiments, the therapeutically effective steady state plasma levels of Compound 1 is provided by administering about 10 mg of Compound 1 or a pharmaceutically acceptable salt thereof twice a day. In further embodiments, the therapeutically effective steady state plasma levels of Compound 1 is provided by administering about 20 mg of Compound 1 or a pharmaceutically acceptable salt thereof once a day.

In some embodiments, the therapeutically effective steady state plasma levels of Compound 1 is provided by administering a daily dose of Compound 1 or a pharmaceutically acceptable salt thereof of about 25 mg. In further embodiments, the therapeutically effective steady state plasma levels of Compound 1 is provided by administering about 25 mg of Compound 1 or a pharmaceutically acceptable salt thereof once a day.

In some embodiments, the therapeutically effective steady state plasma levels of Compound 1 is provided by administering a daily dose of Compound 1 or a pharmaceutically acceptable salt thereof of about 30 mg. In further embodiments, the therapeutically effective steady state plasma levels of Compound 1 is provided by administering about 15 mg of Compound 1 or a pharmaceutically acceptable salt thereof twice a day. In further embodiments, the therapeutically effective steady state plasma levels of Compound 1 is provided by administering about 30 mg of Compound 1 or a pharmaceutically acceptable salt thereof once a day.

In some embodiments, the therapeutically effective steady state plasma levels of Compound 1 is provided by administering a daily dose of Compound 1 or a pharmaceutically acceptable salt thereof of about 35 mg. In further embodiments, the therapeutically effective steady state plasma levels of Compound 1 is provided by administering about 35 mg of Compound 1 or a pharmaceutically acceptable salt thereof once a day.

In some embodiments, the therapeutically effective steady state plasma levels of Compound 1 is provided by administering a daily dose of Compound 1 or a pharmaceutically acceptable salt thereof of about 40 mg. In further embodiments, the therapeutically effective steady state plasma levels of Compound 1 is provided by administering about 20 mg of Compound 1 or a pharmaceutically acceptable salt thereof twice a day. In further embodiments, the therapeutically effective steady state plasma levels of Compound 1 is provided by administering about 40 mg of Compound 1 or a pharmaceutically acceptable salt thereof once a day.

In some embodiments, the therapeutically effective steady state plasma levels of Compound 1 is provided by administering a daily dose of Compound 1 or a pharmaceutically acceptable salt thereof of about 45 mg. In further embodiments, the therapeutically effective steady state plasma levels of Compound 1 is provided by administering about 45 mg of Compound 1 or a pharmaceutically acceptable salt thereof once a day.

In some embodiments, the therapeutically effective steady state plasma levels of Compound 1 is provided by administering a daily dose of Compound 1 or a pharmaceutically acceptable salt thereof of about 50 mg. In further embodiments, the therapeutically effective steady state plasma levels of Compound 1 is provided by administering about 25 mg of Compound 1 or a pharmaceutically acceptable salt thereof twice a day. In further embodiments, the therapeutically effective steady state plasma levels of Compound 1 is provided by administering about 50 mg of Compound 1 or a pharmaceutically acceptable salt thereof once a day.

In some embodiments, the therapeutically effective steady state plasma levels of Compound 1 is provided by administering a daily dose of Compound 1 or a pharmaceutically acceptable salt thereof of about 55 mg. In further embodiments, the therapeutically effective steady state plasma levels of Compound 1 is provided by administering about 55 mg of Compound 1 or a pharmaceutically acceptable salt thereof once a day.

In some embodiments, the therapeutically effective steady state plasma levels of Compound 1 is provided by administering a daily dose of Compound 1 or a pharmaceutically acceptable salt thereof of about 60 mg. In further embodiments, the therapeutically effective steady state plasma levels of Compound 1 is provided by administering about 30 mg of Compound 1 or a pharmaceutically acceptable salt thereof twice a day. In further embodiments, the therapeutically effective steady state plasma levels of Compound 1 is provided by administering about 60 mg of Compound 1 or a pharmaceutically acceptable salt thereof once a day.

In some embodiments, the present methods provide mean steady state AUC₀₋₂₄ₕ (expressed in terms of ng*hr/mL) levels of Compound 1 that correlate to one or more statistically significant therapeutic effects. In some embodiments, the therapeutically effective mean steady state AUC₀₋₂₄ₕ levels of Compound 1 provided by the methods of the present invention range from about 50 ng*hr/mL to about 3000 ng*hr/mL, including about 50 ng*hr/mL, 100 ng*hr/mL, 150 ng*hr/mL, 200 ng*hr/mL, 250 ng*hr/mL, 300 ng*hr/mL, about 400 ng*hr/mL, about 500 ng*hr/mL, about 600 ng*hr/mL, about 700 ng*hr/mL, about 800 ng*hr/mL, about 900 ng*hr/mL, about 1000 ng*hr/mL, about 1100 ng*hr/mL, about 1200 ng*hr/mL, about 1300 ng*hr/mL, about 1400 ng*hr/mL, about 1500 ng*hr/mL, about 1600 ng*hr/mL, about 1700 ng*hr/mL, about 1800 ng*hr/mL, about 1900 ng*hr/mL, about 2000 ng*hr/mL, about 2100 ng*hr/mL, about 2200 ng*hr/mL, about 2300 ng*hr/mL, about 2400 ng*hr/mL, about 2500 ng*hr/mL, about 2600 ng*hr/mL, about 2700 ng*hr/mL, about 2800 ng*hr/mL, about 2900 ng*hr/mL, and about 3000 ng*hr/mL, including all ranges therebetween.

In some embodiments, the mean steady state AUC₀₋₂₄ₕ levels of Compound 1 is provided by administering a daily dose of Compound 1 or a pharmaceutically acceptable salt thereof of about 15 mg. In further embodiments, the mean steady state AUC₀₋₂₄ₕ levels of Compound 1 is provided by administering about 15 mg of Compound 1 or a pharmaceutically acceptable salt thereof once a day.

In some embodiments, the mean steady state AUC₀₋₂₄ₕ levels of Compound 1 is provided by administering a daily dose of Compound 1 or a pharmaceutically acceptable salt thereof of about 20 mg. In further embodiments, the mean steady state AUC₀₋₂₄ₕ levels of Compound 1 is provided by administering about 10 mg of Compound 1 or a pharmaceutically acceptable salt thereof twice a day. In further embodiments, the mean steady state AUC₀₋₂₄ₕ levels of Compound 1 is provided by administering about 20 mg of Compound 1 or a pharmaceutically acceptable salt thereof once a day.

In some embodiments, the mean steady state AUC₀₋₂₄ₕ levels of Compound 1 is provided by administering a daily dose of Compound 1 or a pharmaceutically acceptable salt thereof of about 25 mg. In further embodiments, the mean steady state AUC₀₋₂₄ₕ levels of Compound 1 is provided by administering about 25 mg of Compound 1 or a pharmaceutically acceptable salt thereof once a day.

In some embodiments, the mean steady state AUC₀₋₂₄ₕ levels of Compound 1 is provided by administering a daily dose of Compound 1 or a pharmaceutically acceptable salt thereof of about 30 mg. In further embodiments, the mean steady state AUC₀₋₂₄ₕ levels of Compound 1 is provided by administering about 15 mg of Compound 1 or a pharmaceutically acceptable salt thereof twice a day. In further embodiments, the mean steady state AUC₀₋₂₄ₕ levels of Compound 1 is provided by administering about 30 mg of Compound 1 or a pharmaceutically acceptable salt thereof once a day.

In some embodiments, the mean steady state AUC₀₋₂₄ₕ levels of Compound 1 is provided by administering a daily dose of Compound 1 or a pharmaceutically acceptable salt thereof of about 35 mg. In further embodiments, the mean steady state AUC₀₋₂₄ₕ levels of Compound 1 is provided by administering about 35 mg of Compound 1 or a pharmaceutically acceptable salt thereof once a day.

In some embodiments, the mean steady state AUC₀₋₂₄ₕ levels of Compound 1 is provided by administering a daily dose of Compound 1 or a pharmaceutically acceptable salt thereof of about 40 mg. In further embodiments, the mean steady state AUC₀₋₂₄ₕ levels of Compound 1 is provided by administering about 20 mg of Compound 1 or a pharmaceutically acceptable salt thereof twice a day. In further embodiments, the mean steady state AUC₀₋₂₄ₕ levels of Compound 1 is provided by administering about 40 mg of Compound 1 or a pharmaceutically acceptable salt thereof once a day.

In some embodiments, the mean steady state AUC₀₋₂₄ₕ levels of Compound 1 is provided by administering a daily dose of Compound 1 or a pharmaceutically acceptable salt thereof of about 45 mg. In further embodiments, the mean steady state AUC₀₋₂₄ₕ levels of Compound 1 is provided by administering about 45 mg of Compound 1 or a pharmaceutically acceptable salt thereof once a day.

In some embodiments, the mean steady state AUC₀₋₂₄ₕ levels of Compound 1 is provided by administering a daily dose of Compound 1 or a pharmaceutically acceptable salt thereof of about 50 mg. In further embodiments, the mean steady state AUC₀₋₂₄ₕ levels of Compound 1 is provided by administering about 25 mg of Compound 1 or a pharmaceutically acceptable salt thereof twice a day. In further embodiments, the mean steady state AUC₀₋₂₄ₕ levels of Compound 1 is provided by administering about 50 mg of Compound 1 or a pharmaceutically acceptable salt thereof once a day.

In some embodiments, the mean steady state AUC₀₋₂₄ₕ levels of Compound 1 is provided by administering a daily dose of Compound 1 or a pharmaceutically acceptable salt thereof of about 55 mg. In further embodiments, the mean steady state AUC₀₋₂₄ₕ levels of Compound 1 is provided by administering about 55 mg of Compound 1 or a pharmaceutically acceptable salt thereof once a day.

In some embodiments, the mean steady state AUC₀₋₂₄ₕ levels of Compound 1 is provided by administering a daily dose of Compound 1 or a pharmaceutically acceptable salt thereof of about 60 mg. In further embodiments, the mean steady state AUC₀₋₂₄ₕ levels of Compound 1 is provided by administering about 30 mg of Compound 1 or a pharmaceutically acceptable salt thereof twice a day. In further embodiments, the mean steady state AUC₀₋₂₄ₕ levels of Compound 1 is provided by administering about 60 mg of Compound 1 or a pharmaceutically acceptable salt thereof once a day.

In some embodiments, the present methods provide steady state plasma Cmax levels (or mean steady state plasma Cmax levels) of Compound 1 that correlate to one or more statistically significant therapeutic effects. In some embodiments, the therapeutically effective steady state plasma Cmax levels (or mean steady state plasma Cmax levels) of Compound 1 provided by the methods of the present invention range from about 5 ng/mL to about 500 ng/mL, including about 5 ng/mL, 10 ng/mL, 20 ng/mL, 30 ng/mL, 40 ng/mL, 50 ng/mL, 60 ng/mL, about 70 ng/mL, about 80 ng/mL, about 90 ng/mL, about 100 ng/mL, about 110 ng/mL, about 120 ng/mL, about 130 ng/mL, about 140 ng/mL, about 150 ng/mL, about 160 ng/mL, about 170 ng/mL about 180 ng/mL, about 190 ng/mL, about 200 ng/mL, about 210 ng/mL, about 220 ng/mL, about 230 ng/mL, about 240 ng/mL, about 250 ng/mL, about 260 ng/mL, about 270 ng/mL about 280 ng/mL, about 290 ng/mL, about 300 ng/mL, about 310 ng/mL, about 320 ng/mL, about 330 ng/mL, about 340 ng/mL, about 350 ng/mL, about 360 ng/mL, about 370 ng/mL about 380 ng/mL, about 390 ng/mL, about 400 ng/mL, about 410 ng/mL, about 420 ng/mL, about 430 ng/mL, about 440 ng/mL, about 150 ng/mL, about 460 ng/mL, about 470 ng/mL about 480 ng/mL, about 490 ng/mL, about 500 ng/mL, about 510 ng/mL about 520 ng/mL, about 530 ng/mL, about 540 ng/mL, about 550 ng/mL, about 560 ng/mL, about 570 ng/mL about 580 ng/mL, about 590 ng/mL and about 600 ng/mL, including all ranges therebetween. In some embodiments, the therapeutically effective steady state plasma Cmax levels (or mean steady state plasma Cmax levels) of Compound 1 provided by the methods of the present invention are from about 100 ng/mL to about 275 ng/mL, including about 110 ng/mL, about 120 ng/mL, about 130 ng/mL, about 140 ng/mL, about 150 ng/mL, about 160 ng/mL, about 170 ng/mL about 180 ng/mL, about 190 ng/mL, about 200 ng/mL, about 210 ng/mL, about 220 ng/mL, about 230 ng/mL, about 240 ng/mL, about 250 ng/mL, about 260 ng/mL, about 270 ng/mL, including all ranges therebetween. In some embodiments, the therapeutically effective steady state plasma Cmax levels (or mean steady state plasma Cmax levels) of Compound 1 provided by the methods of the present invention are from about 125 ng/mL to about 250 ng/mL.

In some embodiments, the therapeutically effective steady state plasma Cmax levels (or mean steady state plasma Cmax levels) of Compound 1 is provided by administering a daily dose of Compound 1 or a pharmaceutically acceptable salt thereof is about 15 mg. In further embodiments, the therapeutically effective steady state plasma Cmax levels (or mean steady state plasma Cmax levels) of Compound 1 is provided by administering about 15 mg of Compound 1 or a pharmaceutically acceptable salt thereof once a day.

In some embodiments, the therapeutically effective steady state plasma Cmax levels (or mean steady state plasma Cmax levels) of Compound 1 is provided by administering a daily dose of Compound 1 or a pharmaceutically acceptable salt thereof is about 20 mg. In further embodiments, the therapeutically effective steady state plasma Cmax levels (or mean steady state plasma Cmax levels) of Compound 1 is provided by administering about 10 mg of Compound 1 or a pharmaceutically acceptable salt thereof twice a day. In further embodiments, the therapeutically effective steady state Cmax plasma levels of Compound 1 is provided by administering about 20 mg of Compound 1 or a pharmaceutically acceptable salt thereof once a day.

In some embodiments, the therapeutically effective steady state plasma Cmax levels (or mean steady state plasma Cmax levels) of Compound 1 is provided by administering a daily dose of Compound 1 or a pharmaceutically acceptable salt thereof is about 25 mg. In further embodiments, the therapeutically effective steady state plasma Cmax levels (or mean steady state plasma Cmax levels) of Compound 1 is provided by administering about 25 mg of Compound 1 or a pharmaceutically acceptable salt thereof once a day.

In some embodiments, the therapeutically effective steady state plasma Cmax levels (or mean steady state plasma Cmax levels) of Compound 1 is provided by administering a daily dose of Compound 1 or a pharmaceutically acceptable salt thereof is about 30 mg. In further embodiments, the therapeutically effective steady state plasma Cmax levels (or mean steady state plasma Cmax levels) of Compound 1 is provided by administering about 15 mg of Compound 1 or a pharmaceutically acceptable salt thereof twice a day. In further embodiments, the therapeutically effective steady state Cmax plasma levels of Compound 1 is provided by administering about 30 mg of Compound 1 or a pharmaceutically acceptable salt thereof once a day.

In some embodiments, the therapeutically effective steady state plasma Cmax levels (or mean steady state plasma Cmax levels) of Compound 1 is provided by administering a daily dose of Compound 1 or a pharmaceutically acceptable salt thereof is about 35 mg. In further embodiments, the therapeutically effective steady state plasma Cmax levels (or mean steady state plasma Cmax levels) of Compound 1 is provided by administering about 35 mg of Compound 1 or a pharmaceutically acceptable salt thereof once a day.

In some embodiments, the therapeutically effective steady state plasma Cmax levels (or mean steady state plasma Cmax levels) of Compound 1 is provided by administering a daily dose of Compound 1 or a pharmaceutically acceptable salt thereof is about 40 mg. In further embodiments, the therapeutically effective steady state plasma Cmax levels (or mean steady state plasma Cmax levels) of Compound 1 is provided by administering about 20 mg of Compound 1 or a pharmaceutically acceptable salt thereof twice a day. In further embodiments, the therapeutically effective steady state Cmax plasma levels of Compound 1 is provided by administering about 40 mg of Compound 1 or a pharmaceutically acceptable salt thereof once a day.

In some embodiments, the therapeutically effective steady state plasma Cmax levels (or mean steady state plasma Cmax levels) of Compound 1 is provided by administering a daily dose of Compound 1 or a pharmaceutically acceptable salt thereof is about 45 mg. In further embodiments, the therapeutically effective steady state plasma Cmax levels (or mean steady state plasma Cmax levels) of Compound 1 is provided by administering about 45 mg of Compound 1 or a pharmaceutically acceptable salt thereof once a day.

In some embodiments, the therapeutically effective steady state plasma Cmax levels (or mean steady state plasma Cmax levels) of Compound 1 is provided by administering a daily dose of Compound 1 or a pharmaceutically acceptable salt thereof is about 50 mg. In further embodiments, the therapeutically effective steady state plasma Cmax levels (or mean steady state plasma Cmax levels) of Compound 1 is provided by administering about 25 mg of Compound 1 or a pharmaceutically acceptable salt thereof twice a day. In further embodiments, the therapeutically effective steady state Cmax plasma levels of Compound 1 is provided by administering about 50 mg of Compound 1 or a pharmaceutically acceptable salt thereof once a day.

In some embodiments, the therapeutically effective steady state plasma Cmax levels (or mean steady state plasma Cmax levels) of Compound 1 is provided by administering a daily dose of Compound 1 or a pharmaceutically acceptable salt thereof is about 55 mg. In further embodiments, the therapeutically effective steady state plasma Cmax levels (or mean steady state plasma Cmax levels) of Compound 1 is provided by administering about 55 mg of Compound 1 or a pharmaceutically acceptable salt thereof once a day.

In some embodiments, the therapeutically effective steady state plasma Cmax levels (or mean steady state plasma Cmax levels) of Compound 1 is provided by administering a daily dose of Compound 1 or a pharmaceutically acceptable salt thereof is about 60 mg. In further embodiments, the therapeutically effective steady state plasma Cmax of Compound 1 is provided by administering about 30 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 60 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 65 mg. In further embodiments, the therapeutically effective steady state plasma Cmax levels (or mean steady state plasma Cmax levels) of Compound 1 is provided by administering about 65 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 70 mg. In further embodiments, the therapeutically effective steady state plasma Cmax levels (or mean steady state plasma Cmax levels) of Compound 1 is provided by administering about 35 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 70 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 75 mg. In further embodiments, the therapeutically effective steady state plasma Cmax levels (or mean steady state plasma Cmax levels) of Compound 1 is provided by administering about 75 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 80 mg. In further embodiments, the therapeutically effective steady state plasma Cmax levels (or mean steady state plasma Cmax levels) of Compound 1 is provided by administering about 40 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 80 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 85 mg. In further embodiments, the therapeutically effective steady state plasma Cmax levels (or mean steady state plasma Cmax levels) of Compound 1 is provided by administering about 85 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 90 mg. In further embodiments, the therapeutically effective steady state plasma Cmax levels (or mean steady state plasma Cmax levels) of Compound 1 is provided by administering about 45 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 90 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 95 mg. In further embodiments, the therapeutically effective steady state plasma Cmax levels (or mean steady state plasma Cmax levels) of Compound 1 is provided by administering about 95 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 100 mg. In further embodiments, the therapeutically effective steady state plasma Cmax levels (or mean steady state plasma Cmax levels) of Compound 1 is provided by administering about 50 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 100 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 105 mg. In further embodiments, the therapeutically effective steady state plasma Cmax levels (or mean steady state plasma Cmax levels) of Compound 1 is provided by administering about 105 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 110 mg. In further embodiments, the therapeutically effective steady state plasma Cmax levels (or mean steady state plasma Cmax levels) of Compound 1 is provided by administering about 55 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 110 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 115 mg. In further embodiments, the therapeutically effective steady state plasma Cmax levels (or mean steady state plasma Cmax levels) of Compound 1 is provided by administering about 115 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 120 mg. In further embodiments, the therapeutically effective steady state plasma Cmax levels (or mean steady state plasma Cmax levels) of Compound 1 is provided by administering about 60 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 120 mg of Compound 1 or a pharmaceutically acceptable salt thereof once a day. In some embodiments, the present methods provide steady state plasma Cmax levels (or mean steady state plasma Cmax levels) of Compound 1 that do not exceed 500 ng/mL. In some embodiments, the therapeutically effective steady state plasma Cmax levels (or mean steady state plasma Cmax levels) of Compound 1 provided by the methods of the present invention do not exceed about 500 ng/mL, including less than about 500 ng/mL, less than about 475 ng/mL, less than about 450 ng/mL, than about 425 ng/mL, less than about 400 ng/mL, less than about 375 ng/mL, than about 350 ng/mL, less than about 325 ng/mL, and less than about 300 ng/mL.

### Examples

The present invention is further illustrated by reference to the following Examples. However, it is noted that these Examples, like the embodiments described above, are illustrative and are not to be construed as restricting the scope of the invention in any way.

### Example 1

Healthy subjects aged 18 to 55 years were treated with an oral suspension of Compound 1 to study the safety, tolerability, pharmacokinetics and pharmacodynamics of Compound 1 in healthy subjects. Dose and dose frequencies were evaluated in order to select a regimen that is suitable for subjects with mood disorders.

### Study Design

The study was a randomized, double-blind, placebo-controlled multiple escalating dose study comprised of 3 cohorts that each received an oral suspension. Each cohort consisted of two groups: one group treated with Compound 1 and another treated with placebo. In each cohort, the ratio of Compound 1-treated subjects to placebo-treated subjects was 3:1.

The Compound 1-treated subjects of Cohort 1 were treated with 15.0 mg of Compound 1 once per day (QD). The Compound 1-treated subjects of Cohort 2 were treated with 30.0 mg of Compound 1 QD. The Compound 1-treated subjects of Cohort 3 were treated with 60.0 mg of Compound 1 QD.

A Food Effect Cohort (Cohort 4) was conducted to assess the effect of food on the PK profile of a single dose of Compound 1 when administered to healthy subjects. The subjects of Cohort 4 were treated with 30 mg of Compound 1 QD.

### Dosing:

Patients in each cohort were treated with Compound 1 for 14 consecutive days, unless dosing was halted by the Safety Review Committee (SRC). The dosing of subjects in each of the cohorts was staggered with the decision to dose escalate based on SRC review of a minimum of 14 days of observation of safety and tolerability data and review of the available plasma PK data from the preceding cohort(s). Thus, dose escalation was predicated on tolerability of the prior cohorts.

Compound 1 was administered under fasted conditions (no food or drink, except water, for at least 10 hours prior to dosing). Immediately after administration of Compound 1, the subject was be administered 240 mL water. No additional fluid intake was allowed until 1 hour after Compound 1 administration.

Cohort 1 subjects received a single 15.0 mg dose of a Compound 1 suspension on the morning of Days 1-14. Cohort 2 subjects received a single 30.0 mg dose of a Compound 1 suspension on the morning of Days 1-14. Cohort 3 subjects received a single 60.0 mg dose of a Compound 1 suspension on the morning of Days 1-14. For all cohorts, the last treatment was administered on the morning of Day 14.

Cohort 4 subjects received a single 30 mg dose of a Compound 1 suspension on Days 1 and 5. The Day 1 dose was administered after a minimum of 10 hour fasting. No additional fluid intake was allowed until 1 hour after drug administration. A standard meal was given at least 4 hours post-dose. The Day 5 dose was administered following a high-fat, high calorie meal. Participants remained at the clinical site for a total of 8 days to complete PK sampling after the second dose.

Blood and urine were obtained during each treatment period at designated times for PK and other analyses (see below). Standard safety assessments were measured during each treatment period.

### Pharmacokinetic (PK) Assessments

PK parameters (e.g., C . . . , T . . . , Tin, AUC, etc.) for healthy patients in each cohort was compared to assess the suitability of Compound 1 suspension for the treatment of MDD. Data were obtained from the blood plasma samples collected from each cohort according to the schedule provided.

Plasma samples were analyzed to determine Compound 1 concentrations using a validated assay method. Pharmacokinetic variables (including but not limited to Cₘₐₓ, Tₘₐₓ and AUC₍₀₋ₗₐₛₜ₎) were calculated using non-compartmental analysis. PK parameters for Compound 1 were derived from the plasma concentration data using non-compartmental analysis with Phoenix™ WinNonlin® v 8.0 (Pharsight Corporation, USA).

### Protocol:

Blood (Cohorts 1-3): For each cohort, blood samples were collected on Days 1, 2, 3, 4, 5, 6 and 14 at the following time points: Day 1 at pre-dose (0 hour), 0.25, 0.5, 0.75, 1, 1.5, 2, 2.5, 3, 4, 6, 8, 12, 16 h post-dose; Day 2 at pre-dose (24 h), Day 3 at pre-dose (48 h), Day 4 at pre-dose (72 h), Day 5 at pre-dose (96 h), Day 6 at pre-dose (120 h); Day 14 at pre-dose 0.25, 0.5, 0.75, 1, 1.5, 2, 2.5, 3, 4, 6, 8, 12, 16, 24, 36, 48, and 72 hours. Trough level blood samples were collected on Days 2, 3, 4, 5, 6 and 14, prior to the morning dose administrations.

The following PK parameters were calculated based on the plasma concentrations of Compound 1: maximum observed concentration (Cmax) on Day 1 and at steady state on Day 15 (Cmax,SS), Time of Cmax (Tmax) and Cmax,SS (Tmax,SS), area under the concentration-time curve through the dosing interval on Day 1 and 15 (AUCtau and AUCSS), total clearance at steady state, measured on Day 15 (CLSS), and volume of distribution at steady state, measured on Day 15 (VSS).

Urine (Cohorts 1-3): Urine was collected/pooled at the following collection windows: Day −1 (6 hours) and at Day 14: (0 to 6 hours), (6 to 12 hours), (12 to 24 hours), and (24-48 hours). Urine samples were analyzed to determine Compound 1 concentrations using a validated assay method. Pooling of urine across patients may be allowed if volumes are not sufficient to allow individual determination.

The following PK parameters were calculated based on the urine concentrations of Compound 1: absolute and cumulative amount of Compound 1 excreted in urine and renal clearance (CLR).

Blood (Cohort 4): Serial blood samples were collected relative to the dosing of Compound 1 at the following time points on both Day 1 and Day 5: Pre-dose (0 hours), 0.25, 0.50, 0.75, 1.00, 1.50, 2.00, 2.50, 3.00, 4.00, 6.00, 8.00, 12.00, 16.00, 24.00, 36.00, 48.00 and 72.00 hours post-dose (f2 min). Urine (Cohort 4): No urine analysis was conducted in Cohort 4.

### Pharmacodynamic Assessment

Pharmacodynamic (PD) effects of first dose and steady state Compound 1 concentrations on wake electroencephalograms (EEGs) was studied. Standard 16 channel continuous EEGs were obtained at the following time points: Day-1, Day 1 (1 h after dosing), and Day 14 (1 h after dosing).

### Safety Assessments/Monitoring

Adverse events (AEs) were monitored throughout the duration of the study.

To monitor for possible adverse events, vital signs, hematology and clinical chemistry laboratory parameters, ECG readings, neurological examination findings, and EEG parameters and abnormal findings were recorded at each visit.

### Statistical Analysis

Descriptive statistics were calculated for plasma and urine PK parameter and concentration data, and summarized by study day and time point. Arithmetic means, coefficient of variation (CV), standard deviation, median, minimum, and maximum values, and number of observations were calculated for all PK parameters and trough concentration data. Except for Tmax, the geometric mean, geometric standard deviation, and geometric CV were provided for all PK parameter and concentration data.

### Cohorts 1-3 Results:

The following pharmacokinetic parameters were determined for each cohort: maximum plasma concentration (Cₘₐₓ, observed, Day 1 only); time to reach the maximum plasma concentration (Tₘₐₓ, observed, Day 1 only); and area under the plasma concentration-time curve from time 0 to 24 h post dose (AUC₀₋ₜₐᵤ).

The following steady state pharmacokinetic (Day 14) parameters were determined: tin: elimination half-life associated with the terminal slope (λz) of the semi-logarithmic drug concentration-time curve, calculated as 0.693/λz; C_{max,ss}: maximum plasma concentration (observed); T_{max,ss}: time to reach the maximum plasma concentration (observed); AUCₛₛ: area under the plasma concentration-time curve from 0 to 24 h post dose; AUC_{inf}: area under the plasma concentration-time curve from 0 to infinite time; C_{avg}: average concentration over dosing interval; CLss/F: steady state clearance; and Vz/F: volume of distribution of terminal phase.

Table 1 shows a summary of the observed PK parameters Day 1.

TABLE 1
Summary of Parameters for Compound 1 (calculated
from Day 1 data for Cohorts 1-3)

| | | | | | Ctrough |
|---|---|---|---|---|---|
| | | Tₘₐₓ | Cₘₐₓ | AUC₀₋ₜₐᵤ | (Day 2) |
| Treatment | Statistic | (h) | (ng/mL) | (h*ng/mL) | (ng/mL) |
| Cohort 1 | Mean | 1.056 | 57.367 | 378.685 | 4.207 |
| | SD | 0.273 | 13.169 | 119.963 | 2.298 |
| | Min | 0.75 | 39.10 | 219.21 | 1.69 |
| | Median | 1.00 | 55.00 | 394.97 | 3.89 |
| | Max | 1.50 | 76.00 | 581.62 | 8.01 |
| | % CV | 25.9 | 23.0 | 31.7 | 54.6 |
| | Geo Mean | 1.027 | 56.016 | 361.762 | 3.716 |
| | CV % g mean | 25.08 | 23.59 | 33.22 | 56.06 |
| Cohort 2 | Mean | 2.028 | 132.511 | 842.141 | 9.353 |
| | SD | 2.323 | 50.359 | 181.943 | 3.3335 |
| | Min | 0.75 | 34.60 | 449.41 | 5.06 |
| | Median | 1.00 | 156.00 | 899.25 | 8.62 |
| | Max | 8.00 | 195.00 | 1030.39 | 14.50 |
| | CV % | 114.6 | 38.0 | 21.6 | 35.7 |
| | Geometric Mean | 1.432 | 120.018 | 820.402 | 8.805 |
| | CV % Geometric | 91.88 | 57.47 | 26.22 | 38.93 |
| | Mean | | | | |
| Cohort 3 | Mean | 1.139 | 363.889 | 2343.762 | 19.024 |
| | SD | 0.435 | 96.801 | 585.039 | 10.024 |
| | Min | 0.75 | 213.00 | 1405.82 | 5.44 |
| | Median | 1.00 | 341.00 | 2356.80 | 20.40 |
| | Max | 2.00 | 511.00 | 3128.23 | 32.30 |
| | CV % | 38.2 | 26.6 | 25.0 | 52.7 |
| | Geometric Mean | 1.074 | 352.127 | 2274.106 | 16.049 |
| | CV % Geometric | 36.72 | 28.13 | 27.16 | 75.74 |
| | Mean | | | | |

Table 2 shows a summary of the observed PK parameters Day 14 for Cohorts 1-3.

TABLE 2
Summary of Parameters for Compound 1 (calculated from Day 14 data)

| | | t_{1/2} | T_{max,ss} | C_{max,ss} | AUCinf | CLss/F | Vz/F | AUCss | C_{avg} |
|---|---|---|---|---|---|---|---|---|---|
| Treatment | Statistic | (h) | (h) | (ng/mL) | (h*ng/mL) | (L/h) | (L) | (h*ng/mL) | (ng/mL) |
| Cohort 1 | Mean | 14.772 | 1.139 | 66.333 | 623.3 | 31.415 | 655.194 | 506.288 | 21.093 |
| | SD | 2.260 | 0.435 | 13.733 | 182.7 | 8.313 | 130.993 | 129.280 | 5.387 |
| | Min | 11.48 | 0.75 | 41.50 | 351 | 19.57 | 464.09 | 306.85 | 12.79 |
| | Median | 15.26 | 1.00 | 66.00 | 595.2 | 30.41 | 656.82 | 493.56 | 20.57 |
| | Max | 17.53 | 2.00 | 91.80 | 1003 | 48.88 | 842.72 | 766.41 | 31.93 |
| | % CV | 15.3 | 38.2 | 20.7 | 29.3 | 26.5 | 20.0 | 25.5 | 25.5 |
| | Geo Mean | 14.613 | 1.074 | 65.010 | 600.228 | 30.497 | 642.968 | 491.871 | 20.497 |
| | CV % Geo Mean | 15.90 | 36.72 | 21.98 | 29.87 | 26.08 | 21.16 | 26.08 | 26.08 |
| Cohort 2 | Mean | 12.701 | 0.964 | 164.286 | 1226.440 | 30.949 | 570.566 | 1016.979 | 35.089 |
| | SD | 1.233 | 0.267 | 46.248 | 284.205 | 7.912 | 180.389 | 222.845 | 7.581 |
| | Min | 10.47 | 0.75 | 103.00 | 805.89 | 23.42 | 405.14 | 646.59 | 18.73 |
| | Median | 12.90 | 1.00 | 150.00 | 1268.36 | 30.12 | 513.72 | 995.94 | 37.47 |
| | Max | 14.19 | 1.50 | 222.00 | 1544.30 | 46.40 | 949.89 | 1280.73 | 42.93 |
| | CV % | 9.7 | 27.7 | 28.2 | 23.2 | 25.6 | 31.6 | 21.9 | 21.6 |
| | Geo Mean | 12.647 | 0.937 | 158.580 | 1196.442 | 30.178 | 550.639 | 994.095 | 34.183 |
| | CV % Geo Mean | 10.11 | 25.67 | 29.64 | 24.88 | 24.02 | 28.09 | 24.02 | 26.22 |
| Cohort 3 | Mean | 12.230 | 0.969 | 355.000 | 2373.932 | 32.142 | 574.599 | 2084.130 | 86.839 |
| | SD | 1.323 | 0.248 | 157.003 | 823.150 | 11.640 | 242.861 | 729.880 | 30.412 |
| | Min | 9.79 | 0.75 | 145.00 | 1368.69 | 17.36 | 302.08 | 1190.49 | 49.60 |
| | Median | 12.42 | 1.00 | 371.50 | 2439.64 | 28.45 | 531.34 | 2109.71 | 87.90 |
| | Max | 13.82 | 1.50 | 565.00 | 3931.46 | 50.40 | 971.28 | 3457.01 | 144.04 |
| | CV % | 10.8 | 25.6 | 44.2 | 34.7 | 36.2 | 42.3 | 35.0 | 35.0 |
| | Geo Mean | 12.164 | 0.944 | 320.410 | 2253.016 | 30.398 | 533.456 | 1973.814 | 82.242 |
| | CV % Geo Mean | 11.36 | 23.83 | 54.42 | 35.80 | 36.78 | 42.61 | 36.78 | 36.78 |

Conclusions: Compound 1 underwent rapid absorption with an approximate proportional increase in Cₘₐₓ and AUC parameters at steady state. Where steady state data was available mean tin was varied between 12.23 and 14.77 h and steady state clearance was determined at approximately 31 L/h.

Rapid absorption was observed with a Tmax occurring within the first 2 h of dosing (Tables 1-2). An accumulation factor of the dosing interval (ratio of AUC_{Day14}/AUC_{Day1}) of approximately 1.25 for Cohort 1 and Cohort 2 (Tables 1 and 2). For Cohort 3, the accumulation factor was 0.89 (Tables 1 and 2). The mean plasma concentration (ng/mL) for the first 24 hours after the initial dose is illustrated in FIG. 2. The mean plasma concentration (ng/mL) for the 24 hours after the last dose is illustrated in FIG. 3.

Mean t1/2 for Cohort 1, Cohort 2 and Cohort 3 was 14.77±2.26 h, 12.70±1.23 h and 12.23±1.32 h, respectively. Steady state clearance was approximately 31 L/h for the three cohorts at Day 14 and mean Vz/F was 655, 570 and 574 L for Cohorts 1, 2 and 3, respectively. A proportional increase in Cavg, AUCss and AUCinf was observed between Cohorts 1 and 3 (Tables 1 and 2).

In Cohorts 1-3s, there were no Serious Adverse Events and no clinically relevant vital sign, ECG, or lab abnormalities. Additionally, several subjects experienced elevated mood at doses of 30 mg and 60 mg

### Cohort 4 Results:

Table 3 shows a summary of the observed PK parameters Days 1 (fasted) and 5 (fed).

TABLE 3

| Compound 1 Mean (SD) | | |
|---|---|---|
| PK Parameters Following | | |
| Oral Administration for | | |
| Food Effect Cohort | Fasted (Day 1) | Fed (Day 1) |
| Parameter | n = 10 | n = 10 |
| Cₘₐₓ (ng/mL) | 84.18 (48.245) | 45.36 (10.74) |
| Tₘₐₓ (h) | 1.00 (0.75, 1.77) | 3.25 (1.00, 6.00) |
| AUC₀₋ₗₐₛₜ (h.ng/mL) | 597.7 (302.85) | 629.9 (183.63) |
| AUC_{inf} (h.ng/mL) | 602.3 (304.84) | 634.2 (185.13) |
| t ½ (h) | 11.22 (1.7749) | 11.26 (0.86406) |
| AUC %_{Extrap} | 0.8702 (0.50874) | 0.6632 (0.28199) |

For Tmax, Median (Minimum, Maximum) values are displayed.

### Conclusions:

The mean plasma concentration (ng/mL) for the first 24 hours after the initial dose for the fed and fasted Cohorts are illustrated in FIG. 4.

Subjects displaying high concentrations following fasted conditions also tended to show higher concentrations (relative to others within the treatment group) following fed conditions. Similarly, subjects with low Compound 1 concentrations following fasted conditions tended to have low Compound 1 concentrations following fed conditions. The t1/2 was similar for both fasted and fed conditions (Table 3).

There was evidence of a food effect for Compound 1 when administered as a single 30 mg dose. Approximately 15 to 20% greater overall exposure as measured by AUC0-last and AUCinf was observed following fed conditions. In addition, Cmax was 1.5 to 1.6 times higher under fasting conditions with a tendency for a shorter Tmax compared to fed conditions.

### Example 2

Female patients with a current diagnosis of major depressive disorder (or symptoms that would likely meet criteria for depression if formally assessed) aged 40 or greater with irregular menses greater than 60 days apart, but less than 365 days apart and an average of 4 or greater vasomotor symptoms per day, were treated with an oral formulation of Compound 1 to study the safety, tolerability, pharmacokinetics, and efficacy of Compound 1 in the treatment of the symptoms of perimenopause.

### Study Design

The study is a two-week open label trial in women that are perimenopausal with depression during which patients were treated with an oral formulation of Compound 1 in adjunct to standard antidepressant treatment.

Screening: Screening assessments include: The Mini International Neuropsychiatric Interview (MINI) to confirm a diagnosis of major depressive disorder, The Hamilton Depression Rating Scale (HAM-D) to gauge the severity of depression, a review of medical records to confirm irregular menses greater than 60 days apart, but less than 365 days apart (to confirm a diagnosis of perimenopause) and an average of 4 or greater vasomotor symptoms per day.

Treatment Period: The treatment period was 14 days in duration. Participants were administered daily doses of Compound 1 (up to 120 mg qPM, including 15 mg once a day, 20 mg once a day, 25 mg once a day, 30 mg once a day, 35 mg once a day, 40 mg once a day, 45 mg once a day, 50 mg once a day, 55 mg once a day, 60 mg once a day, 65 mg once a day, 70 mg once a day, 75 mg once a day, 80 mg once a day, 85 mg once a day, 90 mg once a day, 95 mg once a day, 100 mg once a day, 105 mg once a day, 110 mg once a day, 115 mg once a day and 120 mg once a day) for 14 days.

### Patient Population:

This trial will enroll women 40 years of age and older with a current diagnosis of major depressive disorder, irregular menses greater than 60 days apart, but less than 365 days apart and an average of 4 or greater vasomotor symptoms per day.

Number of Participants: The clinical trial is planned to include approximately 12 participants.

Inclusion Criteria: Women 40 years of age and older, hold a current diagnosis of major depressive disorder (or symptoms that would likely meet criteria for depression if formally assessed) and irregular menses greater than 60 days apart, but less than 365 days apart and an average of 4 or greater vasomotor symptoms per day.

Exclusion Criteria: Ongoing or history of any psychiatric, medical or surgical condition that, in the judgment of the Investigator, might jeopardize the participant's safety or interfere with the absorption, distribution, metabolism or excretion of Compound 1. History of seizures. History of hypercoagulability or venous thromboembolism. Taking hormonal therapy or failed a trial of hormonal therapy for depression.

### Dosing:

Patients were administered daily doses of Compound 1 (up to 120 mg qPM, including 15 mg once a day, 20 mg once a day, 25 mg once a day, 30 mg once a day, 35 mg once a day, 40 mg once a day, 45 mg once a day, 50 mg once a day, 55 mg once a day, 60 mg once a day, 65 mg once a day, 70 mg once a day, 75 mg once a day, 80 mg once a day, 85 mg once a day, 90 mg once a day, 95 mg once a day, 100 mg once a day, 105 mg once a day, 110 mg once a day, 115 mg once a day and 120 mg once a day) for 14 days.

Standard safety assessments were measured during the treatment period.

### Formulation

The Compound 1 drug product will be formulated as a suspension whose composition is summarized in Table 4. The Compound 1 oral suspension is planned to contain from 1 to 20 mg/mL of Compound 1.

TABLE 4
Compound 1 Composition of Drug Product
Suspension, 1 mg/mL to 20 mg/mL

| Ingredient | Purpose | Amount, mg/mL |
|---|---|---|
| Compound 1 | Active | 1-20 mg/mL |
| Hypromellose 2910, 4000cP, | Suspension stabilizer | 5.0 |
| USP | | |
| Poloxamer 188, USP | Dispersing agent | 5.0 |
| Purified Water, USP or higher | Excipient | q.s. |
| quality | | |
| To make | | 1.0 mL |

A placebo to match the Compound 1 oral suspension will be manufactured having substantially the same composition as the active drug product but without Compound 1 (active pharmaceutical ingredient). However, microcrystalline cellulose will be used to mimic the appearance of the suspended Compound 1. The composition of the Placebo is summarized in Table 5.

TABLE 5
Composition of Placebo Drug Product Suspension

| Ingredient | Purpose | Amount, mg/mL |
|---|---|---|
| Microcrystalline Cellulose, NF | API simulant | 5.0-20.0* |
| (MCC) | | |
| Hypromellose 2910, 4000cP, | Suspension | 5.0 |
| USP | stabilizer | |
| Poloxamer 188, USP | Dispersing agent | 5.0 |
| Purified water, USP or higher | Excipient | q.s. |
| quality | | |
| To make | | 1.0 mL |

### Protocol:

### Pharmacodynamic Assessment

The Primary efficacy outcome was measured by the Hamilton Depression Rating Scale (HAM-D). The HAM-D was administered at baseline (Day 1, predose), Day 4, Day 8, Day 15, Day 21 and Day 28.

Secondary outcome measure was by the Clinical Global Impression (CGI), DSM-V MDD Anxious Distress Specifier, the Hamilton Anxiety Rating Scale (HAM-A), the Montgomery-Asberg Depression Rating Scale (MADRS), the Perimenopausal Depression Questionnaire (Meno-D), the Pittsburgh Sleep Quality Index (PSQI), the revised Sabbastberg Sexual Function Scale, the Symptoms of Depression Questionnaire (SDQ), and a vasomotor symptom diary.

HAM-D Response was defined as a reduction from baseline of >50% in total HAM-D score. HAM-D remission was defined as a total HAM-D score of <7.

### Safety Assessments/Monitoring

Adverse events (AEs) were monitored throughout the duration of the study.

During the study, physical examination, vital signs, ECG, and clinical laboratory tests were conducted. The Modified Observers Assessment of Anesthesia and Sedation, (MOASS) and the Columbia Suicide were performed.

### Statistical Analysis

Efficacy analysis: The HAM-D total score and change from baseline values will be summarized by treatment group and time point. In addition, the change from baseline in HAM-D total score will also be analyzed using paired t-tests or similar methods.

### Efficacy Results

Efficacy (as determined by reduction Mean Change from Baseline in HAM-D score and perimenopausal depression-specific symptom endpoints) was observed in a patient administered 60 mg of Compound 1 once a day for 14 days.

Prior to treatment with Compound 1, the patient had a baseline HAM-D score of 28, indicating moderate to severe depression, and met DSM-5 criteria for anxious distress. The patient had a baseline MENO-D score of 28, indicating moderate perimenopausal depression symptoms, and a baseline frequency of hot flushes of 9 per day (5 daytime hot flushes and 4 nighttime hot flushes at baseline assessment.). Before treatment with Compound 1, the patient was treated with the antidepressant fluoxetine 15 mg daily taken for 8 months and the patient's depression symptoms had not demonstrated a sufficient clinical response as determined by the HAM-D score and the clinical evaluation. The clinical evaluation also determined that the patient's hot flush symptoms were not improved with fluoxetine treatment, and that they had been increasing in frequency in the prior month despite ongoing treatment with fluoxetine.

Depression Endpoints: On Day 8, the patient demonstrated an improvement in HAM-D score of 48%. On Day 15, the patient met the HAM-D Response criterion (the patient demonstrated an improvement of 71% from baseline value) and nearly met the HAM-D Remission criterion (the patient's Day 15 HAM-D score was 8). Similar improvements were observed in the MADRS depression assessment and the HAM-A anxiety assessment at Days 8 and 15.

Perimenopausal Depression Endpoints: On Days 8 and 15, the patient demonstrated an improvement in MENO-D of 54% and 57%, respectively, from baseline value. Based on the standards developed for this assessment, the patient's Day 8 and Day 15 MENO-D scores no longer met classification for perimenopausal depression requiring clinical attention. The following improvements in the MENO-D factors were observed at Day 15: Self (63%), Sexual (43%), Somatic (50%), Cognitive (100%) and Sleep (40%). The patient's frequency of hot flushes on the vasomotor symptoms diary demonstrated a 66% decrease by Day 4 and the decrease was maintained throughout the majority of the Compound 1 treatment period. Importantly, the patient reported a decrease in hot flush frequency early in the treatment period (i.e., 4 days after starting treatment). The daytime hot flushes decreased to 0 or 1 per day starting at the Day 3 assessment, and the decrease was maintained throughout the majority of the Compound 1 treatment period. The nighttime hot flushes decreased to between 0 to 2 per night starting at the Day 3 assessment, and the decrease was maintained throughout the rest of the Compound 1 treatment period. In contrast, known non-hormonal therapies for the treatment of perimenopausal vasomotor symptoms (including SSRI and SNRI antidepressants) require a longer treatment duration to achieve a significant reduction in hot flush frequency.

Tolerability: The 60 mg dose of Compound 1 was well-tolerated, and the only adverse event the patient reported was mild epigastric pain after dosing.

PK results: The patient's dose (i.e., 60 mg of Compound 1 administered once a day, in the evening for 14 days) is associated with a mean maximum post-dose concentration of 162 ng/mL, which was the average value observed in six MDD patients treated with this dose.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as acknowledgment or any form of suggestion that they constitute valid prior art or form part of the common general knowledge in any country in the world.

### Embodiments

1. A method of treating the symptoms of perimenopause or postmenopause in a patient in need thereof comprising administering a therapeutically effective amount of a GABA-A receptor PAM.
2. The method of embodiment 1, wherein the patient is in perimenopause according to the Stages of Reproductive Aging Workshop+10 (STRAW+10) Staging System.
3. The method of embodiment 2, wherein the patient is in early menopausal transition, or early perimenopause, according to the STRAW+10 Staging System.
4. The method of embodiment 2, wherein the patient is in late menopausal, or late perimenopause, transition according to the STRAW+10 Staging System.
5. The method of any of embodiments 1-2, wherein the patient is in early postmenopause according to the STRAW+10 Staging System.
6. The method of embodiment 1, wherein the patient is in postmenopause according to the STRAW+10 Staging System.
7. The method of embodiment 1, wherein the patient is in perimenopause according to Female Reproductive Lifecycle and Hormones Questionnaire (FRLHQ).
8. The method of any one of embodiments 1-7, wherein the GABA-A receptor PAM is a neuroactive steroid.
9. The method of embodiment 8, wherein the neuroactive steroid is selected from the group consisting of pregnanolone, allopregnanolone, allotetrahydrodeoxycorticosterone, ganaxolone, alphaxolone, alphadolone, hydroxydione, minaxolone, Althesin, Renanolone, SAGE-324 (Zuranolone), SAGE-217 (3α-hydroxy-3β-methyl-21-(4-cyano-1H-pyrazol-1′-yl)-19-nor-50-pregnan-20-one), and any neuroactive steroid as described in U.S. Publication No. 2017/0240589.
10. The method of embodiment 8, wherein the GABA-A receptor PAM is Compound 1:

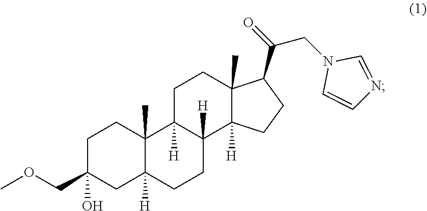

or a pharmaceutically acceptable salt thereof.
11. The method of any one of embodiments 1-7, wherein the GABA-A receptor PAM is etifoxine or a pharmaceutically acceptable salt thereof.
12. The method of any one of embodiments 1-11, wherein the symptoms of perimenopause or postmenopause are selected from the group consisting of vasomotor symptoms, sleep symptoms, cognitive symptoms, sexual symptoms and mood symptoms.
13. The method of embodiment 12, wherein the vasomotor symptoms are selected from the group consisting of hot flushes and night sweats.
14. The method of embodiment 12, wherein the cognitive symptoms are selected from the group consisting of memory loss and difficulty concentrating.
15. The method of embodiment 12, wherein the mood symptoms are selected from the group consisting of perimenopausal depression, irritability and anxiety.
16. The method of any of embodiments 1-15, wherein the GABA-A receptor PAM is orally administered.
17. The method of embodiment 10, wherein the administered daily dose of Compound 1 is from about 5 mg to about 120 mg.
18. The method of embodiment 17, wherein about 45 mg to about 80 mg of Compound 1 or a pharmaceutically acceptable salt thereof is administered per day.
19. The method of embodiment 17, wherein about 45 mg of Compound 1 or a pharmaceutically acceptable salt thereof is administered per day.
20. The method of embodiment 17, wherein about 60 mg of Compound 1 or a pharmaceutically acceptable salt thereof is administered per day.
21. The method of embodiment 17, wherein about 80 mg of Compound 1 or a pharmaceutically acceptable salt thereof is administered per day.
22. The method of embodiment 17, wherein about 100 mg of Compound 1 or a pharmaceutically acceptable salt thereof is administered per day.
23. The method of embodiment 17, wherein about 120 mg of Compound 1 or a pharmaceutically acceptable salt thereof is administered per day.
24. The method of any one of embodiments 17-23 wherein Compound 1 or a pharmaceutically acceptable salt thereof is administered at bedtime.
25. The method of any one of embodiments 17-24, wherein Compound 1 or a pharmaceutically acceptable salt thereof is administered without regard to meals.
26. The method of any one of embodiments 17-25, wherein the method comprises administering Compound 1 or a pharmaceutically acceptable salt thereof for about 1 week, about 2 weeks, about 3 weeks, about 4 weeks, about two months, about three months, about four months, about five months, about six months, about seven months, about eight months, about nine months, about ten months, about eleven months, about twelve months, about 18 months, about 24 months, about 30 months or about 36 months.
27. The method of any one of embodiments 17-26, wherein the method comprises continuous administration of Compound 1 or a pharmaceutically acceptable salt thereof.
28. The method of embodiment 27, wherein the method comprises:
   (a) administering Compound 1 or a pharmaceutically acceptable salt thereof for about 1 week and
   (b) after the administration period (a) not administering Compound 1 or a pharmaceutically acceptable salt thereof for at least 3 weeks.
29. The method of embodiment 27, wherein the method comprises:
   (a) administering Compound 1 or a pharmaceutically acceptable salt thereof for about 3 weeks and
   (b) after the administration period (a) not administering Compound 1 or a pharmaceutically acceptable salt thereof for at least 3 weeks.
30. The method of embodiment 27, wherein the method comprises:
   (a) administering Compound 1 or a pharmaceutically acceptable salt thereof for about 4 weeks and
   (b) after the administration period (a) not administering Compound 1 or a pharmaceutically acceptable salt thereof for at least 3 weeks.
31. The method of any one of embodiments 17-26, wherein the method comprises intermittent administration of Compound 1 or a pharmaceutically acceptable salt thereof.
32. The method of embodiment 31, wherein the intermittent administration comprises:
   (a) administering Compound 1 or a pharmaceutically acceptable salt thereof for a first administration period;
   (b) after the first administration period (a), not administering Compound 1 or a pharmaceutically acceptable salt thereof for a cessation period;
   (c) after the cessation period (b), administering Compound 1 or a pharmaceutically acceptable salt thereof for a second administration period.
33. The method of embodiment 31, wherein the intermittent administration comprises:
   (a) administering Compound 1 or a pharmaceutically acceptable salt thereof for about 1 week;
   (b) after the administration period (a) not administering Compound 1 or a pharmaceutically acceptable salt thereof for about 1 week; and
   (c) after the cessation period (b) administering Compound 1 or a pharmaceutically acceptable salt thereof for about 1 week.
34. The method of embodiment 31, wherein the intermittent administration comprises:
   (a) administering Compound 1 or a pharmaceutically acceptable salt thereof for about 2 weeks;
   (b) after the administration period (a) not administering Compound 1 or a pharmaceutically acceptable salt thereof for about 2 weeks; and
   (c) after the cessation period (b) administering Compound 1 or a pharmaceutically acceptable salt thereof for about 2 weeks.
35. The method of any one of embodiments 31-34, further comprising not administering Compound 1 or a pharmaceutically acceptable salt thereof for one or more additional cessation periods.
36. The method of any one of embodiments 31-35, further comprising administering Compound 1 or a pharmaceutically acceptable salt thereof for one or more additional administration periods.
37. The method of any one embodiments 32 and 35-36, wherein the first administration period is about one week, about two weeks, about three weeks, about four weeks, about five weeks, about six weeks, about seven weeks, or about eight weeks.
38. The method of any one of embodiments 32 and 35-37, wherein the cessation period is about one week, about two weeks, about three weeks, about four weeks, about five weeks, about six weeks, about seven weeks, or about eight weeks.
39. The method of any one of embodiments 32 and 35-38, wherein the second administration period is about one week, about two weeks, about three weeks, about four weeks, about five weeks, about six weeks, about seven weeks, or about eight weeks.
40. The method of any one embodiments 32 and 35-36, wherein the first administration period is about one week; the cessation period is about three weeks; and the second administration period is about one week.
41. The method of any one embodiments 32 and 35-36, wherein the first administration period is about two weeks; the first cessation period is about two weeks; the second administration period is about one week; the second cessation period is about one week and the third administration period is about one week.
42. The method of any one of embodiments 32-41, wherein intermittent administration period is about one month, about two months, about three months, about four months, about five months, about six months, about seven months, about eight months, about nine months, about ten months, about eleven months, about twelve months, about 18 months, about 24 months, about 30 months or about 36 months.
43. The method of any one of embodiments 17-42, further comprising titrating the dose of Compound 1 or a pharmaceutically acceptable salt thereof for at least one week until a maintenance dose is achieved in the patient.
44. The method of embodiment 43, wherein the initial dose of Compound 1 or a pharmaceutically acceptable salt thereof is from about 15 mg to about 45 mg.
45. The method of any one of embodiments 43-44, wherein the maintenance dose of Compound 1 or a pharmaceutically acceptable salt thereof is from about 45 mg to about 80 mg.
46. The method of any one of embodiments 43-45, wherein the initial dose is administered for one week and the maintenance dose is administered for at least one week.
47. The method of any of embodiments 17-26, wherein the method comprises:
   (a) administering a loading dose of Compound 1 or a pharmaceutically acceptable salt thereof to a patient in need thereof and
   (b) administering a maintenance dose of Compound 1 or a pharmaceutically acceptable salt thereof.
48. The method of embodiment 47, wherein the loading dose is administered for about 1 day, about 2 days, about 3 days, about 4 days, about 5 days, about 6 days, about 7 days, about 8 days, about 9 days, about 10 days, about 11 days, about 12 days, about 13 days or about 14 days.
49. The method of embodiment of any one of embodiments 47-48, wherein the loading dose of Compound 1 or a pharmaceutically acceptable salt thereof is about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg.
50. The method of embodiment of any one of embodiments 43 and 47-49, wherein the maintenance dose is administered for about 1 month, about 2 months, about 3 months, about 4 months, about 5 months, about 6 months, about 7 months, about 8 months, about 9 months, about 10 months, about 11 months, about 12 months, about 18 months, about 24 months, about 30 months, or about 36 months.
51. The method of embodiment of any one of embodiments 43 and 47-50, wherein the maintenance dose of Compound 1 or a pharmaceutically acceptable salt thereof is about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg.
52. The method of embodiment of any one of embodiments 47-51, wherein the method further comprises a cessation period after administration of the loading dose and prior to administration of the maintenance dose.
53. The method of embodiment 52, wherein the cessation period is about one day, about two days, about three days, about four days, about five days, about six days, or about seven days.
54. The method of embodiment 52, wherein the cessation period is about one week, about two weeks, about three weeks, about four weeks, about five weeks, about six weeks, about seven weeks, or about eight weeks.
55. The method of any one of embodiments 1-54, wherein after said administering, the patient experiences a substantial reduction in the vasomotor symptoms compared to prior to said administering.
56. The method of embodiment 55, wherein after said administering, the patient experiences a reduction of the vasomotor symptoms that is characterized by an at least 10% decline in the patient's daily total number of hot flushes.
57. The method of embodiment 55, wherein after said administering, the patient experiences a reduction of the vasomotor symptoms that is characterized by an at least 10% decline in the average daily intensity of the patient's hot flushes.
58. The method of embodiment 55, wherein after said administering, the patient experiences a reduction of the vasomotor symptoms that is characterized by an at least 10% decline in the patient's daily daytime number of hot flushes.
59. The method of embodiment 55, wherein after said administering, the patient experiences a reduction of the vasomotor symptoms that is characterized by an at least 10% decline in the average daily daytime intensity of the patient's hot flushes.
60. The method of embodiment 55, wherein after said administering, the patient experiences a reduction of the vasomotor symptoms that is characterized by an at least 10% decline in the patient's daily nighttime number of hot flushes.
61. The method of embodiment 55, wherein after said administering, the patient experiences a reduction of the vasomotor symptoms that is characterized by an at least 10% decline in the average daily nighttime intensity of the patient's hot flushes.
62. The method of embodiment 55, wherein after said administering, the patient experiences a reduction of the vasomotor symptoms that is characterized by an at least 10% decline in the average daily number of the patient's night sweats.
63. The method of embodiment 55, wherein after said administering, the patient experiences a reduction of the vasomotor symptoms that is characterized by an at least 10% decline in the average daily intensity of the patient's night sweats.
64. The method of any one of embodiments 1-63, wherein after said administering, the patient experiences a substantial reduction in the anxiety symptoms of perimenopause or menopause compared to prior to said administering.
65. The method of embodiment 64, wherein after said administering, the patient experiences a substantial reduction the patient experiences a substantial reduction in the anxiety symptoms that is characterized by at least about a 20% decline in total GAD-7 value compared to prior to the treatment.
66. The method of embodiment 64, wherein after said administering, the patient experiences a reduction in the anxiety symptoms that is characterized by an at least two point decline in total GAD-7 value compared to prior to the treatment.
67. The method of embodiment 64, wherein after said administering, the patient experiences a reduction in the anxiety symptoms that is characterized by an at least two point decline in total GAD-7 value compared to prior to the treatment.
68. The method of embodiment 64, wherein after said administering, the patient experiences a reduction in the anxiety symptoms that is characterized by an at least one category change in GAD-7 severity classification compared to prior to the treatment.
69. The method of any one of embodiments 1-68 wherein after said administering, the patient experiences a substantial reduction in perimenopausal depression compared to prior to said administering.
70. The method of embodiment 69, wherein after said administering, the patient experiences a reduction of perimenopausal depression that is characterized by an at least about a 20% decline in MENO-D value.
71. The method of embodiment 69, wherein after said administering, the patient experiences a reduction of perimenopausal depression that is characterized by an at least two point decline in MENO-D value.
72. The method of embodiment 69, wherein after said administering, the patient experiences a reduction of perimenopausal depression that is characterized by at least 1 point decline in at least one MENO-D factor value selected from Self, Sexual, Somatic, Cognitive and Sleep compared to prior to the treatment.
73. The method of embodiment 69, wherein after said administering, the patient experiences a reduction of perimenopausal depression that is characterized by an at least two point decline in total Hamilton Depression Rating Scale (HAM-D) value.
74. The method of embodiment 69, wherein after said administering, the patient experiences a reduction of perimenopausal depression that is characterized by an at least 20% reduction in HAM-D value.
75. The method of embodiment 69, wherein after said administering, the patient experiences a reduction of perimenopausal depression that is characterized by an at least one category change in HAM-D severity classification.
76. The method of embodiment 69, wherein after said administering, the patient experiences a reduction of perimenopausal depression that is characterized by an at least two point decline in Montgomery Asberg Depression Rating Scale (MADRS) value.
77. The method of embodiment 69, wherein after said administering, the patient experiences a reduction of perimenopausal depression that is characterized by an at least 20% reduction in MADRS value.
78. The method of any one of embodiments 1-77, wherein after said administering, the patient experiences a substantial reduction in the sexual symptoms of perimenopause or menopause compared to prior to said administering.
79. The method of embodiment 78, wherein after said administering, the patient experiences a reduction of the sexual symptoms of perimenopause or menopause that is characterized by an at least 20% reduction in revised Sabbatsberg Sexual Self-Rating Scale (SRS) Value.
80. The method of embodiment 78, wherein after said administering, the patient experiences a substantial reduction in the sexual symptoms that is characterized by at least about a 10% decline in at least one SRS subscale value selected from sexual interest, sexual activity, satisfaction of sexual life, experience of sexual pleasure, orgasm capacity, and sexual relevancy compared to prior to the treatment.
81. The method of embodiment 78, wherein after said administering, the patient experiences a reduction of sexual symptoms that is characterized by an at least two point decline in total SRS value.
82. The method of any one of embodiments 1-81, wherein after said administering, the patient experiences a substantial reduction in the mood symptoms of perimenopause or menopause compared to prior to said administering.
83. The method of embodiment 82, wherein the after said administering, the patient experiences a substantial reduction in the mood symptoms that is characterized by at least about a 20% decline in total Profile of Mood States (POMS-SF) value compared to prior to the treatment.
84. The method of embodiment 81, wherein after said administering, the patient experiences a substantial reduction in the mood symptoms that is characterized by an at least two point decline in total POMS-SF value.
85. The method of embodiment 84, wherein the after said administering, the patient experiences a substantial reduction in the mood symptoms that is characterized by at least about a 10% decline in at least one POMS-SF subscale value selected from Fatigue-Inertia, Vigor-Activity, Tension-Anxiety, Depression-Dejection, Anger-Hostility and Confusion-Bewilderment compared to prior to the treatment.
86. The method of any one of embodiments 17-85, wherein Compound 1 is a pharmaceutically acceptable salt.
87. The method of embodiment 86, wherein the pharmaceutically acceptable salt is selected from the group consisting of hydrobromide, citrate, malate, mesylate, phosphate, and tartrate.
88. The method of any one of embodiments 17-87, wherein the administration to a patient in need thereof provides a mean steady state blood plasma AUC₀₋₂₄ₕ from about 500 to about 2500 ng*h/ml of Compound 1.
89. The method of any one of embodiments 17-88, wherein the administration to a patient in need thereof provides a steady state blood plasma Cmax from about 50 ng/mL to about 400 ng/ml of the Compound 1.
90. The method of any one of embodiments 17-89, wherein the administration to a patient in need thereof provides a steady state blood plasma Cmax from about 125 ng/mL to about 250 ng/ml of the Compound 1.
91. The method of any one of embodiments 17-89, wherein the administration to a patient in need thereof provides a steady state blood plasma Cmax that does not exceed 500 ng/ml of Compound 1.
92. The composition of any one of embodiments 1-91, wherein the composition is an oral dosage form.
93. The method of any one of embodiments 17-92, wherein Compound 1 is in the form of an extended release oral dosage form.
94. The method of any one of embodiments 1-93, further comprising administering one or more one additional therapeutic agents.
95. The method of embodiment 94, wherein the additional therapeutic agent is an antidepressant.
96. The method of embodiment 95, wherein the additional antidepressant is selected from the group consisting of selective serotonin reuptake inhibitors, serotonin norepinephrine reuptake inhibitors, tricyclic antidepressants, monoamine oxidase inhibitors, mirtazapine bupropion, lamotrigine atypical antipsychotics, ketamine, esketamine, and antiepileptic drugs.
97. The method of embodiment 95, wherein the selective serotonin reuptake inhibitor is selected from the group consisting of fluoxetine, escitalopram, citalopram, sertraline, and paroxetine.
98. The method of embodiment 95, wherein the serotonin norepinephrine reuptake inhibitor is selected from the group consisting of venlafaxine and duloxetine.
99. The method of embodiment 95, wherein the serotonin tricyclic antidepressant is selected from the group consisting of amitriptyline, imipramine, and nortriptyline.
100. The method of embodiment 95, wherein the monoamine oxidase inhibitor is selected from the group consisting of phenelzine and tranylcypromine.
101. The method of embodiment 95, wherein the atypical antipsychotic is selected from the group consisting of lurasidone, aripiprazole, brexpiprazole, risperidone, olanzapine, quetiapine, ziprasidone, clozapine, iloperidone, paliperidone, asenapine and olanzapine/fluoxetine.
102. The method of embodiment 94, wherein the additional therapeutic agent is a hormone replacement therapy.
103. The method of embodiment 102, wherein the hormone replacement therapy is selected from an estrogen and a progestin, or a combination thereof.
104. The method of embodiment 103, wherein the estrogen is selected from the group consisting of estradiol, synthetic conjugated estrogens, estradiol valerate, estradiol acetate, esterified estrogen, and estropipate.
105. The method of embodiment 103, wherein the progestin is selected from the group consisting of progesterone and medroxyprogesterone acetate.
106. The method of embodiment 103, wherein the combination of estrogen and progestin are selected from the group consisting of estradiol/norethindrone acetate, estradiol/drospirenone, estradiol/levonrgestrel, estradiol/norethindrone acetate, norethindrone acetate/ethinyl estradiol, estradiol/norgestimate, and conjugated estrogen/medroxyprogesterone.
107. The method of embodiment 94, wherein the additional therapeutic agent is one or more selective estrogen receptor modulator (SERM).
108. The method of embodiment 107, wherein the SERM is ospemifene.

What is claimed is:

1. A method of treating the symptoms of perimenopause or postmenopause in a patient in need thereof comprising administering a therapeutically effective amount of a GABA-A receptor PAM.

2. The method of claim 1, wherein the patient is in perimenopause according to the Stages of Reproductive Aging Workshop+10 (STRAW+10) Staging System.

3. The method of claim 2, wherein the patient is in early menopausal transition, or early perimenopause, according to the STRAW+10 Staging System.

4. The method of claim 2, wherein the patient is in late menopausal, or late perimenopause, transition according to the STRAW+10 Staging System.

5. The method of any of claims 1-2, wherein the patient is in early postmenopause according to the STRAW+10 Staging System.

6. The method of claim 1, wherein the patient is in postmenopause according to the STRAW+10 Staging System.

7. The method of claim 1, wherein the patient is in perimenopause according to Female Reproductive Lifecycle and Hormones Questionnaire (FRLHQ).

8. The method of any one of claims 1-7, wherein the GABA-A receptor PAM is a neuroactive steroid.

9. The method of claim 8, wherein the neuroactive steroid is selected from the group consisting of pregnanolone, allopregnanolone, allotetrahydrodeoxycorticosterone, ganaxolone, alphaxolone, alphadolone, hydroxydione, minaxolone, Althesin, Renanolone, SAGE-324 (Zuranolone), SAGE-217 (3α-hydroxy-3β-methyl-21-(4-cyano-1H-pyrazol-1′-yl)-19-nor-5β-pregnan-20-one), and any neuroactive steroid as described in U.S. Publication No. 2017/0240589.

10. The method of claim 8, wherein the GABA-A receptor PAM is Compound 1: 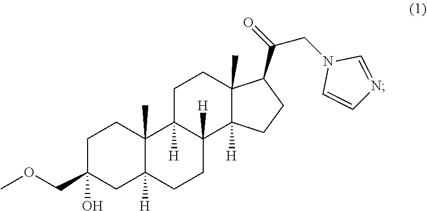
or a pharmaceutically acceptable salt thereof.

11. The method of any one of claims 1-7, wherein the GABA-A receptor PAM is etifoxine or a pharmaceutically acceptable salt thereof.

12. The method of any one of claims 1-11, wherein the symptoms of perimenopause or postmenopause are selected from the group consisting of vasomotor symptoms, sleep symptoms, cognitive symptoms, sexual symptoms and mood symptoms.

13. The method of claim 12, wherein the vasomotor symptoms are selected from the group consisting of hot flushes and night sweats.

14. The method of claim 12, wherein the cognitive symptoms are selected from the group consisting of memory loss and difficulty concentrating.

15. The method of claim 12, wherein the mood symptoms are selected from the group consisting of perimenopausal depression, irritability and anxiety.

16. The method of any of claims 1-15, wherein the GABA-A receptor PAM is orally administered.

17. The method of claim 10, wherein the administered daily dose of Compound 1 is from about 5 mg to about 120 mg.

18. The method of claim 17, wherein about 45 mg to about 80 mg of Compound 1 or a pharmaceutically acceptable salt thereof is administered per day.

19. The method of claim 17, wherein about 45 mg of Compound 1 or a pharmaceutically acceptable salt thereof is administered per day.

20. The method of claim 17, wherein about 60 mg of Compound 1 or a pharmaceutically acceptable salt thereof is administered per day.

21. The method of claim 17, wherein about 80 mg of Compound 1 or a pharmaceutically acceptable salt thereof is administered per day.

22. The method of claim 17, wherein about 100 mg of Compound 1 or a pharmaceutically acceptable salt thereof is administered per day.

23. The method of claim 17, wherein about 120 mg of Compound 1 or a pharmaceutically acceptable salt thereof is administered per day.

24. The method of any one of claims 17-23 wherein Compound 1 or a pharmaceutically acceptable salt thereof is administered at bedtime.

25. The method of any one of claims 17-24, wherein Compound 1 or a pharmaceutically acceptable salt thereof is administered without regard to meals.

26. The method of any one of claims 17-25, wherein the method comprises administering Compound 1 or a pharmaceutically acceptable salt thereof for about 1 week, about 2 weeks, about 3 weeks, about 4 weeks, about two months, about three months, about four months, about five months, about six months, about seven months, about eight months, about nine months, about ten months, about eleven months, about twelve months, about 18 months, about 24 months, about 30 months or about 36 months.

27. The method of any one of claims 17-26, wherein the method comprises continuous administration of Compound 1 or a pharmaceutically acceptable salt thereof.

28. The method of claim 27, wherein the method comprises:
(a) administering Compound 1 or a pharmaceutically acceptable salt thereof for about 1 week and
(b) after the administration period (a) not administering Compound 1 or a pharmaceutically acceptable salt thereof for at least 3 weeks.

29. The method of claim 27, wherein the method comprises:
(a) administering Compound 1 or a pharmaceutically acceptable salt thereof for about 3 weeks and
(b) after the administration period (a) not administering Compound 1 or a pharmaceutically acceptable salt thereof for at least 3 weeks.

30. The method of claim 27, wherein the method comprises:
(a) administering Compound 1 or a pharmaceutically acceptable salt thereof for about 4 weeks and
(b) after the administration period (a) not administering Compound 1 or a pharmaceutically acceptable salt thereof for at least 3 weeks.

31. The method of any one of claims 17-26, wherein the method comprises intermittent administration of Compound 1 or a pharmaceutically acceptable salt thereof.

32. The method of claim 31, wherein the intermittent administration comprises:
(a) administering Compound 1 or a pharmaceutically acceptable salt thereof for a first administration period;
(b) after the first administration period (a), not administering Compound 1 or a pharmaceutically acceptable salt thereof for a cessation period;
(c) after the cessation period (b), administering Compound 1 or a pharmaceutically acceptable salt thereof for a second administration period.

33. The method of claim 31, wherein the intermittent administration comprises:
(a) administering Compound 1 or a pharmaceutically acceptable salt thereof for about 1 week;
(b) after the administration period (a) not administering Compound 1 or a pharmaceutically acceptable salt thereof for about 1 week; and
(c) after the cessation period (b) administering Compound 1 or a pharmaceutically acceptable salt thereof for about 1 week.

34. The method of claim 31, wherein the intermittent administration comprises:
(a) administering Compound 1 or a pharmaceutically acceptable salt thereof for about 2 weeks;
(b) after the administration period (a) not administering Compound 1 or a pharmaceutically acceptable salt thereof for about 2 weeks; and
(c) after the cessation period (b) administering Compound 1 or a pharmaceutically acceptable salt thereof for about 2 weeks.

35. The method of any one of claims 31-34, further comprising not administering Compound 1 or a pharmaceutically acceptable salt thereof for one or more additional cessation periods.

36. The method of any one of claims 31-35, further comprising administering Compound 1 or a pharmaceutically acceptable salt thereof for one or more additional administration periods.

37. The method of any one claims 32 and 35-36, wherein the first administration period is about one week, about two weeks, about three weeks, about four weeks, about five weeks, about six weeks, about seven weeks, or about eight weeks.

38. The method of any one of claims 32 and 35-37, wherein the cessation period is about one week, about two weeks, about three weeks, about four weeks, about five weeks, about six weeks, about seven weeks, or about eight weeks.

39. The method of any one of claims 32 and 35-38, wherein the second administration period is about one week, about two weeks, about three weeks, about four weeks, about five weeks, about six weeks, about seven weeks, or about eight weeks.

40. The method of any one claims 32 and 35-36, wherein the first administration period is about one week; the cessation period is about three weeks; and the second administration period is about one week.

41. The method of any one claims 32 and 35-36, wherein the first administration period is about two weeks; the first cessation period is about two weeks; the second administration period is about one week; the second cessation period is about one week and the third administration period is about one week.

42. The method of any one of claims 32-41, wherein intermittent administration period is about one month, about two months, about three months, about four months, about five months, about six months, about seven months, about eight months, about nine months, about ten months, about eleven months, about twelve months, about 18 months, about 24 months, about 30 months or about 36 months.

43. The method of any one of claims 17-42, further comprising titrating the dose of Compound 1 or a pharmaceutically acceptable salt thereof for at least one week until a maintenance dose is achieved in the patient.

44. The method of claim 43, wherein the initial dose of Compound 1 or a pharmaceutically acceptable salt thereof is from about 15 mg to about 45 mg.

45. The method of any one of claims 43-44, wherein the maintenance dose of Compound 1 or a pharmaceutically acceptable salt thereof is from about 45 mg to about 80 mg.

46. The method of any one of claims 43-45, wherein the initial dose is administered for one week and the maintenance dose is administered for at least one week.

47. The method of any of claims 17-26, wherein the method comprises:
(a) administering a loading dose of Compound 1 or a pharmaceutically acceptable salt thereof to a patient in need thereof and
(b) administering a maintenance dose of Compound 1 or a pharmaceutically acceptable salt thereof.

48. The method of claim 47, wherein the loading dose is administered for about 1 day, about 2 days, about 3 days, about 4 days, about 5 days, about 6 days, about 7 days, about 8 days, about 9 days, about 10 days, about 11 days, about 12 days, about 13 days or about 14 days.

49. The method of claim of any one of claims 47-48, wherein the loading dose of Compound 1 or a pharmaceutically acceptable salt thereof is about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg.

50. The method of claim of any one of claims 43 and 47-49, wherein the maintenance dose is administered for about 1 month, about 2 months, about 3 months, about 4 months, about 5 months, about 6 months, about 7 months, about 8 months, about 9 months, about 10 months, about 11 months, about 12 months, about 18 months, about 24 months, about 30 months, or about 36 months.

51. The method of claim of any one of claims 43 and 47-50, wherein the maintenance dose of Compound 1 or a pharmaceutically acceptable salt thereof is about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg.

52. The method of claim of any one of claims 47-51, wherein the method further comprises a cessation period after administration of the loading dose and prior to administration of the maintenance dose.

53. The method of claim 52, wherein the cessation period is about one day, about two days, about three days, about four days, about five days, about six days, or about seven days.

54. The method of claim 52, wherein the cessation period is about one week, about two weeks, about three weeks, about four weeks, about five weeks, about six weeks, about seven weeks, or about eight weeks.

55. The method of any one of claims 1-54, wherein after said administering, the patient experiences a substantial reduction in the vasomotor symptoms compared to prior to said administering.

56. The method of claim 55, wherein after said administering, the patient experiences a reduction of the vasomotor symptoms that is characterized by an at least 10% decline in the patient's daily total number of hot flushes.

57. The method of claim 55, wherein after said administering, the patient experiences a reduction of the vasomotor symptoms that is characterized by an at least 10% decline in the average daily intensity of the patient's hot flushes.

58. The method of claim 55, wherein after said administering, the patient experiences a reduction of the vasomotor symptoms that is characterized by an at least 10% decline in the patient's daily daytime number of hot flushes.

59. The method of claim 55, wherein after said administering, the patient experiences a reduction of the vasomotor symptoms that is characterized by an at least 10% decline in the average daily daytime intensity of the patient's hot flushes.

60. The method of claim 55, wherein after said administering, the patient experiences a reduction of the vasomotor symptoms that is characterized by an at least 10% decline in the patient's daily nighttime number of hot flushes.

61. The method of claim 55, wherein after said administering, the patient experiences a reduction of the vasomotor symptoms that is characterized by an at least 10% decline in the average daily nighttime intensity of the patient's hot flushes.

62. The method of claim 55, wherein after said administering, the patient experiences a reduction of the vasomotor symptoms that is characterized by an at least 10% decline in the average daily number of the patient's night sweats.

63. The method of claim 55, wherein after said administering, the patient experiences a reduction of the vasomotor symptoms that is characterized by an at least 10% decline in the average daily intensity of the patient's night sweats.

64. The method of any one of claims 1-63, wherein after said administering, the patient experiences a substantial reduction in the anxiety symptoms of perimenopause or menopause compared to prior to said administering.

65. The method of claim 64, wherein after said administering, the patient experiences a substantial reduction the patient experiences a substantial reduction in the anxiety symptoms that is characterized by at least about a 20% decline in total GAD-7 value compared to prior to the treatment.

66. The method of claim 64, wherein after said administering, the patient experiences a reduction in the anxiety symptoms that is characterized by an at least two point decline in total GAD-7 value compared to prior to the treatment.

67. The method of claim 64, wherein after said administering, the patient experiences a reduction in the anxiety symptoms that is characterized by an at least two point decline in total GAD-7 value compared to prior to the treatment.

68. The method of claim 64, wherein after said administering, the patient experiences a reduction in the anxiety symptoms that is characterized by an at least one category change in GAD-7 severity classification compared to prior to the treatment.

69. The method of any one of claims 1-68 wherein after said administering, the patient experiences a substantial reduction in perimenopausal depression compared to prior to said administering.

70. The method of claim 69, wherein after said administering, the patient experiences a reduction of perimenopausal depression that is characterized by an at least about a 20% decline in MENO-D value.

71. The method of claim 69, wherein after said administering, the patient experiences a reduction of perimenopausal depression that is characterized by an at least two point decline in MENO-D value.

72. The method of claim 69, wherein after said administering, the patient experiences a reduction of perimenopausal depression that is characterized by at least 1 point decline in at least one MENO-D factor value selected from Self, Sexual, Somatic, Cognitive and Sleep compared to prior to the treatment.

73. The method of claim 69, wherein after said administering, the patient experiences a reduction of perimenopausal depression that is characterized by an at least two point decline in total Hamilton Depression Rating Scale (HAM-D) value.

74. The method of claim 69, wherein after said administering, the patient experiences a reduction of perimenopausal depression that is characterized by an at least 20% reduction in HAM-D value.

75. The method of claim 69, wherein after said administering, the patient experiences a reduction of perimenopausal depression that is characterized by an at least one category change in HAM-D severity classification.

76. The method of claim 69, wherein after said administering, the patient experiences a reduction of perimenopausal depression that is characterized by an at least two point decline in Montgomery Asberg Depression Rating Scale (MADRS) value.

77. The method of claim 69, wherein after said administering, the patient experiences a reduction of perimenopausal depression that is characterized by an at least 20% reduction in MADRS value.

78. The method of any one of claims 1-77, wherein after said administering, the patient experiences a substantial reduction in the sexual symptoms of perimenopause or menopause compared to prior to said administering.

79. The method of claim 78, wherein after said administering, the patient experiences a reduction of the sexual symptoms of perimenopause or menopause that is characterized by an at least 20% reduction in revised Sabbatsberg Sexual Self-Rating Scale (SRS) Value.

80. The method of claim 78, wherein after said administering, the patient experiences a substantial reduction in the sexual symptoms that is characterized by at least about a 10% decline in at least one SRS subscale value selected from sexual interest, sexual activity, satisfaction of sexual life, experience of sexual pleasure, orgasm capacity, and sexual relevancy compared to prior to the treatment.

81. The method of claim 78, wherein after said administering, the patient experiences a reduction of sexual symptoms that is characterized by an at least two point decline in total SRS value.

82. The method of any one of claims 1-81, wherein after said administering, the patient experiences a substantial reduction in the mood symptoms of perimenopause or menopause compared to prior to said administering.

83. The method of claim 82, wherein the after said administering, the patient experiences a substantial reduction in the mood symptoms that is characterized by at least about a 20% decline in total Profile of Mood States (POMS-SF) value compared to prior to the treatment.

84. The method of claim 81, wherein after said administering, the patient experiences a substantial reduction in the mood symptoms that is characterized by an at least two point decline in total POMS-SF value.

85. The method of claim 84, wherein the after said administering, the patient experiences a substantial reduction in the mood symptoms that is characterized by at least about a 10% decline in at least one POMS-SF subscale value selected from Fatigue-Inertia, Vigor-Activity, Tension-Anxiety, Depression-Dejection, Anger-Hostility and Confusion-Bewilderment compared to prior to the treatment.

86. The method of any one of claims 17-85, wherein Compound 1 is a pharmaceutically acceptable salt.

87. The method of claim 86, wherein the pharmaceutically acceptable salt is selected from the group consisting of hydrobromide, citrate, malate, mesylate, phosphate, and tartrate.

88. The method of any one of claims 17-87, wherein the administration to a patient in need thereof provides a mean steady state blood plasma AUC₀₋₂₄ₕ from about 500 to about 2500 ng*h/ml of Compound 1.

89. The method of any one of claims 17-88, wherein the administration to a patient in need thereof provides a steady state blood plasma Cmax from about 50 ng/mL to about 400 ng/ml of the Compound 1.

90. The method of any one of claims 17-89, wherein the administration to a patient in need thereof provides a steady state blood plasma Cmax from about 125 ng/mL to about 250 ng/ml of the Compound 1.

91. The method of any one of claims 17-89, wherein the administration to a patient in need thereof provides a steady state blood plasma Cmax that does not exceed 500 ng/ml of Compound 1.

92. The composition of any one of claims 1-91, wherein the composition is an oral dosage form.

93. The method of any one of claims 17-92, wherein Compound 1 is in the form of an extended release oral dosage form.

94. The method of any one of claims 1-93, further comprising administering one or more one additional therapeutic agents.

95. The method of claim 94, wherein the additional therapeutic agent is an antidepressant.

96. The method of claim 95, wherein the additional antidepressant is selected from the group consisting of selective serotonin reuptake inhibitors, serotonin norepinephrine reuptake inhibitors, tricyclic antidepressants, monoamine oxidase inhibitors, mirtazapine bupropion, lamotrigine atypical antipsychotics, ketamine, esketamine, and antiepileptic drugs.

97. The method of claim 95, wherein the selective serotonin reuptake inhibitor is selected from the group consisting of fluoxetine, escitalopram, citalopram, sertraline, and paroxetine.

98. The method of claim 95, wherein the serotonin norepinephrine reuptake inhibitor is selected from the group consisting of venlafaxine and duloxetine.

99. The method of claim 95, wherein the serotonin tricyclic antidepressant is selected from the group consisting of amitriptyline, imipramine, and nortriptyline.

100. The method of claim 95, wherein the monoamine oxidase inhibitor is selected from the group consisting of phenelzine and tranylcypromine.

101. The method of claim 95, wherein the atypical antipsychotic is selected from the group consisting of lurasidone, aripiprazole, brexpiprazole, risperidone, olanzapine, quetiapine, ziprasidone, clozapine, iloperidone, paliperidone, asenapine and olanzapine/fluoxetine.

102. The method of claim 94, wherein the additional therapeutic agent is a hormone replacement therapy.

103. The method of claim 102, wherein the hormone replacement therapy is selected from an estrogen and a progestin, or a combination thereof.

104. The method of claim 103, wherein the estrogen is selected from the group consisting of estradiol, synthetic conjugated estrogens, estradiol valerate, estradiol acetate, esterified estrogen, and estropipate.

105. The method of claim 103, wherein the progestin is selected from the group consisting of progesterone and medroxyprogesterone acetate.

106. The method of claim 103, wherein the combination of estrogen and progestin are selected from the group consisting of estradiol/norethindrone acetate, estradiol/drospirenone, estradiol/levonrgestrel, estradiol/norethindrone acetate, norethindrone acetate/ethinyl estradiol, estradiol/norgestimate, and conjugated estrogen/medroxyprogesterone.

107. The method of claim 94, wherein the additional therapeutic agent is one or more selective estrogen receptor modulator (SERM).

108. The method of claim 107, wherein the SERM is ospemifene.
